(19) United States
(12) Patent Application Publication
(10) Pub. No.: US 2022/0251591 A1
(43) Pub. Date: Aug. 11, 2022

(54) ABIOTIC STRESS TOLERANT PLANTS AND METHODS

(71) Applicants: PIONEER OVERSEAS CORPORATION, JOHNSTON, IA (US); SINOBIOWAY BIO-AGRICULTURE GROUP CO. LTD., BEIJING (CN)

(72) Inventors: GUIHUA LU, SAN DIEGO, CA (US); GUANGWU CHEN, BEIJING (CN); RONGRONG JIAO, BEIJING (CN); AIFEN LIU, LAIYANG CITY (CN); GUANFAN MAO, BEIJING (CN); CHANGGUI WANG, BEIJING (CN); GUOKUI WANG, BEIJING (CN); YU ZHANG, BEIJING (CN)

(73) Assignees: PIONEER OVERSEAS CORPORATION, JOHNSTON, IA (US); SINOBIOWAY BIO-AGRICULTURE GROUP CO. LTD., BEIJING (CN)

(21) Appl. No.: 17/629,336

(22) PCT Filed: Jul. 31, 2019
(86) PCT No.: PCT/CN2019/098525

§ 371 Date: Jan. 21, 2022

(51) Int. Cl. C12N 15/82; C07K 14/415

(52) U.S. Cl. CPC C12N 15/8273; C07K 14/415

(57) ABSTRACT

Isolated polynucleotides and polypeptides, and recombinant DNA constructs are useful for conferring improved drought tolerance and yield. Compositions (such as plants or seeds) comprise these recombinant DNA constructs; and methods utilize these recombinant DNA constructs. The recombinant DNA constructs comprise a polynucleotide operably linked to a promoter that is functional in a plant, wherein said polynucleotides encode drought tolerance polypeptides.

### FIELD

The field of the disclosure relates to plant breeding and genetics and, particularly, relates to improving tolerance to abiotic stress in plants.

### BACKGROUND

Stresses to plants may be caused by both biotic and abiotic agents. For example, biotic causes of stress include infection with pathogen, insect feeding, and parasitism by another plant such as mistletoe. Abiotic stresses include, for example, excessive or insufficient available water, temperature extremes, and synthetic chemicals such as herbicides.

Abiotic stress is the primary cause of crop loss worldwide, causing average yield losses of more than 50% for major crops (Boyer, J. S. (1982) Science 218:443-448; Bray, E. A. et al. (2000) In Biochemistry and Molecular Biology of Plants, edited by Buchannan, B. B. et al., Amer. Soc. Plant Biol., pp. 1158-1249; Mushtaq et al. (2018) Journal of Plant Physiology 224-225: 156-162).

Accordingly, there is a need to develop compositions and methods that increase tolerance to abiotic stress in plants. This invention provides such compositions and methods.

### SUMMARY

The following embodiments are among those encompassed by the disclosure:

In one embodiment, the present disclosure includes an isolated polynucleotide, encoding a polypeptide with an amino acid sequence of at least 90% sequence identity to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158, wherein increased expression of the polynucleotide in a plant enhances drought tolerance. In certain embodiments, the isolated polynucleotide encodes the amino acid sequence of SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158. In certain embodiments, the isolated polynucleotide comprises the nucleotide sequence of SEQ ID NO: 1, 2, 4, 6, 7, 9, 10, 12, 13, 15, 16, 18, 19, 21, 22, 24, 25, 27, 28, 30, 31, 33, 34, 36, 37, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157. In certain embodiments, increased expression of the polynucleotide in a plant enhances grain yield under drought conditions.

The present disclosure also provides a recombinant DNA construct comprising an isolated polynucleotide operably linked to at least one heterologous regulatory element, wherein the polynucleotide encodes a polypeptide with an amino acid sequence of at least 90% sequence identity to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158.

The present disclosure further provides a modified plant or seed having increased expression or activity of at least one polynucleotide encoding a polypeptide with an amino acid sequence of at least 90% sequence identity to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158. In certain embodiments, the modified plant or seed comprises in its genome a recombinant DNA construct comprising a polynucleotide operably linked to at least one heterologous regulatory element, wherein the polynucleotide encodes a polypeptide with an amino acid sequence of at least 90% sequence identity to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158. In certain embodiments, the modified plant exhibits improved drought tolerance and/or increased grain yield when grown under drought conditions compared to a control plant.

In certain embodiments, the modified plant or seed comprises a targeted genetic modification at a genomic locus comprising a polynucleotide encoding a polypeptide with an amino acid sequence of at least 90% sequence identity to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158, wherein the targeted genetic modification increases the expression and/or activity of the polypeptide. In certain embodiments, the modified plant exhibits improved drought tolerance, such as increased grain yield when grown under drought conditions compared to a control plant.

In certain embodiments, the plant is selected from the group consisting of rice, maize, soybean, sunflower, sorghum, canola, wheat, alfalfa, cotton, barley, millet, sugar cane and switchgrass.

Also provided are methods for increasing drought tolerance in a plant, the method comprising increasing the expression of at least one polynucleotide encoding a polypeptide with an amino acid sequence of at least 90% sequence identity to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158 in the plant, wherein the plant exhibits increased drought tolerance when compared to the control plant.

In certain embodiments, the method for increasing drought tolerance comprises: (a) introducing into a regenerable plant cell a recombinant DNA construct comprising a polynucleotide operably linked to at least one heterologous regulatory element, wherein the polynucleotide encodes a polypeptide having an amino acid sequence of at least 80% sequence identity, when compared to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158; and (b) generating the plant, wherein the plant comprises in its genome the recombinant DNA construct.

In certain embodiments, the method for increasing drought tolerance comprises: (a) introducing into a regenerable plant cell a targeted genetic modification at a genomic locus comprising a polynucleotide encoding a polypeptide having an amino acid sequence of at least 80% sequence identity, when compared to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158; and (b) generating the plant, wherein the plant comprises in its genome the introduced genetic modification and has increased expression and/or activity of the polypeptide. In certain embodiments, the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), an engineered site-specific meganucleases, or an Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at 80% sequence identity, when compared to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158.

### BRIEF DESCRIPTION OF THE DRAWINGS AND SEQUENCE LISTING

The disclosure can be more fully understood from the following detailed description and the accompanying Sequence Listing which form a part of this application. The sequence descriptions and sequence listing attached here to comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

TABLE 1
Sequence Listing Descriptions

| | | SEQ ID NO: | SEQ ID NO: |
|---|---|---|---|
| Source/Plant species | Clone Designation | (Nucleotide) | (Amino Acid) |
| Oryza sativa | OsFBX335 | 1, 2, 4 | 3, 5 |
| Oryza sativa | OsNADH1 | 6, 7 | 8 |
| Oryza sativa | OsPUF1 | 9, 10 | 11 |
| Oryza sativa | OsPK1 | 12, 13 | 14 |
| Oryza sativa | OsFAO1 | 15, 16 | 17 |
| Oryza sativa | OsDN-DTP17 | 18, 19 | 20 |
| Oryza sativa | OsCuAO1 | 21, 22 | 23 |
| Oryza sativa | OsDRH7 | 24, 25 | 26 |
| Oryza sativa | OsDN-DTP9 | 27, 28 | 29 |
| Oryza sativa | OsDN-DTP18 | 30, 31 | 32 |
| Oryza sativa | OsSCP52 | 33, 34 | 35 |
| Oryza sativa | OsDN-DTP19 | 36, 37 | 38 |
| Artificial | Gene clone primers | 39-62 | n/a |
| Artificial | RT-PCR primers | 63-68 | n/a |
| Oryza sativa | OsFBX335Paralog | 69 | 70 |
| Zea mays | OsFBX335 Homolog | 71 | 72 |
| Sorghum bicolor | OsFBX335 Homolog | 73 | 74 |
| Oryza sativa | OsNADH1 Paralog | 75 | 76 |
| Zea mays | OsNADH1 Homolog | 77 | 78 |
| Arabidopsis thaliana | OsNADH1 Homolog | 79 | 80 |
| Oryza sativa | OsPUF1 Paralog | 81 | 82 |
| Zea mays | OsPUF1 Homolog | 83 | 84 |
| Sorghum bicolor | OsPUF1 Homolog | 85 | 86 |
| Arabidopsis thaliana | OsPUF1 Homolog | 87 | 88 |
| Glycine max | OsPUF1 Homolog | 89 | 90 |
| Oryza sativa | OsPK1 Paralog | 91 | 92 |
| Zea mays | OsPK1 Homolog | 93 | 94 |
| Sorghum bicolor | OsPK1 Homolog | 95 | 96 |
| Arabidopsis thaliana | OsPK1 Homolog | 97 | 98 |
| Glycine max | OsPK1 Homolog | 99 | 100 |
| Oryza sativa | OsFAO1 Paralog | 101 | 102 |
| Zea mays | OsFAO1 Homolog | 103 | 104 |
| Sorghum bicolor | OsFAO1 Homolog | 105 | 106 |
| Arabidopsis thaliana | OsFAO1 Homolog | 107 | 108 |
| Glycine max | OsFAO1 Homolog | 109 | 110 |
| Oryza sativa | OsDN-DTP17 Paralog | 111 | 112 |
| Zea mays | OsDN-DTP17 Homolog | 113 | 114 |
| Sorghum bicolor | OsDN-DTP17 Homolog | 115 | 116 |
| Arabidopsis thaliana | OsDN-DTP17 Homolog | 117 | 118 |
| Glycine max | OsDN-DTP17 Homolog | 119 | 120 |
| Oryza sativa | OsCuAO1 Paralog | 121 | 122 |
| Zea mays | OsCuAO1 Homolog | 123 | 124 |
| Sorghum bicolor | OsCuAO1 Homolog | 125 | 126 |
| Oryza sativa | OsDRH7 Paralog | 127 | 128 |
| Zea mays | OsDRH7 Homolog | 129 | 130 |
| Sorghum bicolor | OsDRH7 Homolog | 131 | 132 |
| Arabidopsis thaliana | OsDRH7 Homolog | 133 | 134 |
| Glycine max | OsDRH7 Homolog | 135 | 136 |
| Oryza sativa | OsDN-DTP9 Paralog | 137 | 138 |
| Zea mays | OsDN-DTP18Homolog | 139 | 140 |
| Sorghum bicolor | OsDN-DTP18 Homolog | 141 | 142 |
| Arabidopsis thaliana | OsDN-DTP18 Homolog | 143 | 144 |
| Glycine max | OsDN-DTP18 Homolog | 145 | 146 |
| Oryza sativa | OsSCP52 Paralog | 147 | 148 |
| Zea mays | OsSCP52 Homolog | 149 | 150 |
| Sorghum bicolor | OsSCP52 Homolog | 151 | 152 |
| Arabidopsis thaliana | OsSCP52 Homolog | 153 | 154 |
| Glycine max | OsSCP52 Homolog | 155 | 156 |
| Oryza sativa | OsDN-DTP19 Paralog | 157 | 158 |

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

### Definitions

As used herein, “increased drought tolerance” of a plant refers to any measurable improvement in a physiological or physical characteristic, such as yield, as measured relative to a reference or control plant when grown under drought conditions. Typically, when a plant comprising a recombinant DNA construct or DNA modification in its genome exhibits increased drought tolerance relative to a reference or control plant, the reference or control plant does not comprise in its genome the recombinant DNA construct or DNA modification.

“Agronomic characteristic” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A “control”, “control plant” or “control plant cell” or the like provides a reference point for measuring changes in phenotype of a subject plant or plant cell in which genetic alteration, such as transformation, has been affected as to a gene of interest. For example, a control plant may be a plant having the same genetic background as the subject plant except for the genetic alteration that resulted in the subject plant or cell.

“Plant” includes reference to whole plants, plant organs, plant tissues, seeds and plant cells and progeny of the same. Plant cells include, without limitation, cells from seeds, suspension cultures, embryos, meristematic regions, callus tissues, leaves, roots, shoots, gametophytes, sporophytes, pollen, and microspores.

“Progeny” comprises any subsequent generation of a plant.

“Modified plant” includes reference to a plant which comprises within its genome a heterologous polynucleotide or modified gene or promoter. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, and “nucleic acid fragment” are used interchangeably and refer to a polymer of RNA or DNA that is single- or double-stranded, optionally containing synthetic, non-natural or altered nucleotide bases. Nucleotides (usually found in their 5′-monophosphate form) are referred to by their single-letter designation as follows: “A” for adenylate or deoxyadenylate, “C” for cytidylate or deoxycytidylate, and “G” for guanylate or deoxyguanylate for RNA or DNA, respectively; “U” for uridylate; “T” for deoxythymidylate; “R” for purines (A or G); “Y” for pyrimidines (C or T); “K” for G or T; “H” for A or C or T; “I” for inosine; and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, and sulfation, gamma-carboxylation of glutamic acid residues, hydroxylation and ADP-ribosylation.

“Recombinant DNA construct” refers to a combination of nucleic acid fragments that are not normally found together in nature. Accordingly, a recombinant DNA construct may comprise regulatory elements and coding sequences that are derived from different sources, or regulatory elements and coding sequences derived from the same source but arranged in a manner different than that normally found in nature.

“Regulatory elements” refer to nucleotide sequences located upstream (5′ non-coding sequences), within, or downstream (3′ non-coding sequences) of a coding sequence, and influencing the transcription, RNA processing or stability, or translation of the associated coding sequence. Regulatory elements may include, but are not limited to, promoters, translation leader sequences, introns, and poly-adenylation recognition sequences. The terms “regulatory sequence” and “regulatory element” and “regulatory region” are used interchangeably herein.

“Promoter” refers to a nucleic acid fragment capable of controlling transcription of another nucleic acid fragment. “Promoter functional in a plant” is a promoter capable of controlling transcription of genes in plant cells whether its origin is from a plant cell or not. “Tissue-specific promoter” and “tissue-preferred promoter” refers to a promoter that is expressed predominantly but not necessarily exclusively in one tissue or organ, but that may also be expressed in one specific cell or cell type. “Developmentally regulated promoter” is a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

As used herein “increased”, “increase”, or the like refers to any detectable increase in an experimental group (e.g., plant with a DNA modification described herein) as compared to a control group (e.g., wild-type plant that does not comprise the DNA modification). Accordingly, increased expression of a protein comprises any detectable increase in the total level of the protein in a sample and can be determined using routine methods in the art such as, for example, Western blotting and ELISA.

As used herein, “yield” refers to the amount of agricultural production harvested per unit of land and may include reference to bushels per acre or kilograms per mu of a crop at harvest, as adjusted for grain moisture (e.g., typically 15% for maize, 13.5% for rice). Grain moisture is measured in the grain at harvest. The adjusted test weight of grain is determined to be the weight in pounds per bushel or grams per plant, adjusted for grain moisture level at harvest.

As used herein, “sequence identity” or “identity” in the context of two polynucleotides or polypeptide sequences refer to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” is calculated by determining the number of positions at which the identical nucleic acid base or amino acid residue occurs in both sequences to yield the number of matched positions, dividing the number of matched positions by the total number of positions in the window of comparison, and multiplying the result by 100.

Unless stated otherwise, multiple alignments of the sequences provided herein are performed using the Clustal V method of alignment (Higgins and Sharp. (1989) CABIOS. 5:151-153) with the default parameters (GAP PENALTY=10, GAP LENGTH PENALTY=10). Default parameters for pairwise alignments and calculation of percent identity of amino acid sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

### Compositions:

### A. Polynucleotides and Polypeptides

The present disclosure provides polynucleotides encoding the following polypeptides: FBX335 (OsFBX335—F-box domain containing protein); NADH1 (respiratory-chain NADH dehydrogenase, 49 KD subunit family protein); DRH7(DEAD-box ATP-dependent RNA helicase 7); PUF1 (plant protein of unknown function domain containing protein); PK1 (pyruvate kinase, putative); D N-DTP17 (expressed protein); CuAO1 (copper amine oxidase, putative); FAO1 (alcohol oxidase, putative); DN-DTP9 (conserved hypothetical protein); DN-DTP18 (expressed protein); SCP52 (OsSCP52—Putative Serine Carboxypeptidase homologue); and DN-DTP19 (expressed protein).

One aspect of the disclosure provides a polynucleotide encoding a polypeptide comprising an amino acid sequence that is at least 80% identical (e.g. 81%, 82%, 83%, 84%, 85%, 86%, 87%, 88%, 89%, 90%, 91%, 92%, 93%, 94%, 95%, 96%, 97%, 98%, 99%, or 100%) to the amino acid sequence of any one of SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158.

“OsFBX335” refers to a rice polypeptide that confers drought tolerance phenotype when overexpressed. The OsFBX335 polypeptides (SEQ ID NOs: 3 and 5) are encoded by the coding sequences (CDS) (SEQ ID NOs: 2 and 4) or nucleotide sequence (SEQ ID NO: 1) at rice gene locus LOC_Os09g32860.1, which is annotated as “OsFBX335-F-box domain containing protein, expressed” in TIGR. “FBX335 polypeptide” refers herein to the OsFBX335 polypeptide and its paralogs (e.g., SEQ ID NO: 70 encoded by SEQ ID NO: 69) or its homologs from other organisms, such as maize (SEQ ID NO: 72 encoded by SEQ ID NO: 71) and sorghum (SEQ ID NO: 74 encoded by SEQ ID NO: 73).

“OsNADH1” refers to a rice polypeptide that confers drought tolerance phenotype when overexpressed. The OsNADH1 polypeptide (SEQ ID NO: 8) is encoded by the coding sequence (CDS) (SEQ ID NO: 7) or nucleotide sequence (SEQ ID NO: 6) at rice gene locus LOC_Os07g41300.1, which is annotated as “respiratory-chain NADH dehydrogenase, 49 KD subunit family protein, expressed” in TIGR. “NADH1 polypeptide” refers herein to the OsNADH1 polypeptide and its paralogs (SEQ ID NO: 76 encoded by SEQ ID NO: 75) or homologs from other organisms, such as maize (SEQ ID NO: 78 encoded by SEQ ID NO: 77) and Arabidopsis (SEQ ID NO: 80 encoded by SEQ ID NO: 79).

“OsPUF1” refers to a rice polypeptide that confers drought tolerance phenotype when overexpressed. The OsPUF1 polypeptide (SEQ ID NO: 11) is encoded by the coding sequence (CDS) (SEQ ID NO: 10) or nucleotide sequence (SEQ ID NO: 9) at rice gene locus LOC_Os03g19700.1, which is annotated as “plant protein of unknown function domain containing protein, expressed” in TIGR. “PUF1 polypeptide” refers herein to the OsPUF1 polypeptide and its paralogs (SEQ ID NO: 82 encoded by SEQ ID NO: 81) or homologs from other organisms, such as maize (SEQ ID NO: 84 encoded by SEQ ID NO: 83); sorghum (SEQ ID NO: 86 encoded by SEQ ID NO: 85); Arabidopsis (SEQ ID NO: 88 encoded by SEQ ID NO: 87); and soybean (SEQ ID NO: 90 encoded by SEQ ID NO: 89).

“OsPK1” refers to a rice polypeptide that confers drought tolerance phenotype when overexpressed. The OsPK1 polypeptide (SEQ ID NO: 14) is encoded by the coding sequence (CDS) (SEQ ID NO: 13) or nucleotide sequence (SEQ ID NO: 12) at rice gene locus LOC_Os01g47080.1, which is annotated as “pyruvate kinase, putative, expressed” in TIGR. “PK1 polypeptide” refers herein to the OsPK1 polypeptide and its paralogs (e.g., SEQ ID NO: 92 encoded by SEQ ID NO: 91) or homologs from other organisms, such as maize (SEQ ID NO: 94 encoded by SEQ ID NO: 93), sorghum (SEQ ID NO: 96 encoded by SEQ ID NO: 95), Arabidopsis (SEQ ID NO: 98 encoded by SEQ ID NO: 97), and soybean (SEQ ID NO: 100 encoded by SEQ ID NO: 99).

“OsFAO1” refers to a rice polypeptide that confers drought tolerance phenotype when overexpressed. The OsFAO1 polypeptide (SEQ ID NO: 17) is encoded by the coding sequence (CDS) (SEQ ID NO: 16) or nucleotide sequence (SEQ ID NO: 15) at rice gene locus LOC_Os02g40840.1, which is annotated as “alcohol oxidase, putative, expressed” in TIGR. “FAO1 polypeptide” refers herein to the OsFAO1 polypeptide and its paralogs (e.g., SEQ ID NO: 102 encoded by SEQ ID NO: 101) or homologs from other organisms, such as maize (SEQ ID NO: 104 encoded by SEQ ID NO: 103), sorghum (SEQ ID NO: 106 encoded by SEQ ID NO: 105), Arabidopsis (SEQ ID NO: 108 encoded by SEQ ID NO: 107), and soybean (SEQ ID NO: 110 encoded by SEQ ID NO: 109).

“OsDN-D TP17” refers to a rice polypeptide that confers drought tolerance phenotype when overexpressed. The OsDN-D TP17 polypeptide (SEQ ID NO: 20) is encoded by the coding sequence (CDS) (SEQ ID NO: 19) or nucleotide sequence (SEQ ID NO: 18) at rice gene locus LOC_Os01g19400.1, which is annotated as “expressed protein” in TIGR. “DN-DTP17 polypeptide” refers herein to the OsDN-DTP17 polypeptide and its paralogs (e.g., SEQ ID NO: 112 encoded by SEQ ID NO: 111) or homologs from other organisms, such as maize (SEQ ID NO: 114 encoded by SEQ ID NO: 113), sorghum (SEQ ID NO: 116 encoded by SEQ ID NO: 115), Arabidopsis (SEQ ID NO: 118 encoded by SEQ ID NO: 117), and soybean (SEQ ID NO: 120 encoded by SEQ ID NO: 119).

“OsCuAO1” refers to a rice polypeptide that confers drought tolerance phenotype when overexpressed. The OsCuAO1 polypeptide (SEQ ID NO: 23) is encoded by the coding sequence (CDS) (SEQ ID NO: 22) or nucleotide sequence (SEQ ID NO: 21) at rice gene locus LOC_Os04g39120.1, which is annotated as “copper amine oxidase, putative, expressed” in TIGR. “CuAO1 polypeptide” refers herein to the OsCuAO1 polypeptide and its paralogs (e.g., SEQ ID NO: 122 encoded by SEQ ID NO: 121) or homologs from other organisms, such as maize (SEQ ID NO: 124 encoded by SEQ ID NO: 123) and sorghum (SEQ ID NO: 126 encoded by SEQ ID NO: 125).

“OsDRH7” refers to a rice polypeptide that confers drought tolerance phenotype when overexpressed. The OsDRH7 polypeptide (SEQ ID NO: 26) is encoded by the coding sequence (CDS) (SEQ ID NO: 25) or nucleotide sequence (SEQ ID NO: 24) at rice gene locus LOC_Os09g34910.1, which is annotated as “DEAD-box ATP-dependent RNA helicase 7, putative, expressed” in TIGR. “DRH7polypeptide” refers herein to the OsDRH7 polypeptide and its paralogs (e.g., SEQ ID NO: 128 encoded by SEQ ID NO: 127) or homologs from other organisms, such as maize (SEQ ID NO: 130 encoded by SEQ ID NO:129), sorghum (SEQ ID NO: 132 encoded by SEQ ID NO: 131), Arabidopsis (SEQ ID NO: 134 encoded by SEQ ID NO: 133), and soybean (SEQ ID NO: 136 encoded by SEQ ID NO:135).

“OsDN-D TP9” refers to a rice polypeptide that confers drought tolerance phenotype when overexpressed. The OsDN-D TP9 polypeptide (SEQ ID NO: 29) is encoded by the coding sequence (CDS) (SEQ ID NO: 28) or nucleotide sequence (SEQ ID NO: 27) at rice gene locus LOC_Os07g45810.1, which is annotated as “conserved hypothetical protein” in TIGR. “DN-DTP9 polypeptide” refers herein to the OsDN-D TP9 polypeptide and its paralogs (e.g., SEQ ID NO: 138 encoded by SEQ ID NO: 137) or homologs from other organisms.

“OsDN-D TP18” refers to a rice polypeptide that confers drought tolerance phenotype when overexpressed. The OsDN-D TP18 polypeptide (SEQ ID NO: 32) is encoded by the coding sequence (CDS) (SEQ ID NO: 31) or nucleotide sequence (SEQ ID NO: 30) at rice gene locus LOC_Os09g12780.1, which is annotated as “expressed protein” in TIGR. “DN-DTP18 polypeptide” refers herein to the OsDN-DTP18 polypeptide and its paralogs or homologs from other organisms, such as maize (SEQ ID NO: 140 encoded by SEQ ID NO:139), sorghum (SEQ ID NO: 142 encoded by SEQ ID NO: 141), Arabidopsis (SEQ ID NO: 144 encoded by SEQ ID NO: 143), and soybean (SEQ ID NO: 146 encoded by SEQ ID NO:145).

“OsSCP52” refers to a rice polypeptide that confers drought tolerance phenotype when overexpressed. The OsSCP52 polypeptide (SEQ ID NO: 35) is encoded by the coding sequence (CDS) (SEQ ID NO: 34) or nucleotide sequence (SEQ ID NO: 33) at rice gene locus LOC_Os11g24320.1, which is annotated as “OsSCP52-Putative Serine Carboxypeptidase homologue” in TIGR. “SCP52 polypeptide” refers herein to the OsSCP52 polypeptide and its paralogs (e.g., SEQ ID NO: 148 encoded by SEQ ID NO: 147) or homologs from other organisms, such as maize (SEQ ID NO: 150 encoded by SEQ ID NO: 149), sorghum (SEQ ID NO: 152 encoded by SEQ ID NO: 151), Arabidopsis (SEQ ID NO: 154 encoded by SEQ ID NO: 153), and soybean (SEQ ID NO: 156 encoded by SEQ ID NO: 155).

“OsDN-D TP19” refers to a rice polypeptide that confers drought tolerance phenotype when overexpressed. The OsDN-D TP19 polypeptide (SEQ ID NO: 38) is encoded by the coding sequence (CDS) (SEQ ID NO: 37) or nucleotide sequence (SEQ ID NO: 36) at rice gene locus LOC_Os01g11360.1, which is annotated as “expressed protein” in TIGR. “DN-DTP19 polypeptide” refers herein to the OsDN-DTP19 polypeptide and its paralogs (e.g., SEQ ID NO: 158 encoded by SEQ ID NO: 157) or homologs from other organisms.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

### B. Recombinant DNA Constructs

Also provided are recombinant DNA constructs comprising any of the polynucleotides described herein. In certain embodiments, the recombinant DNA construct further comprises at least one regulatory element. In certain embodiments the at least one regulatory element is a heterologous regulatory element. In certain embodiments, the at least one regulatory element of the recombinant DNA construct comprises a promoter. In certain embodiments, the promoter is a heterologous promoter.

A number of promoters can be used in recombinant DNA constructs of the present disclosure. The promoters can be selected based on the desired outcome, and may include constitutive, tissue-specific, inducible, or other promoters for expression in the host organism.

A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

A tissue-specific or developmentally-regulated promoter is a DNA sequence which regulates the expression of a DNA sequence selectively in the cells/tissues of a plant, such as in those cells/tissues critical to tassel development, seed set, or both, and which usually limits the expression of such a DNA sequence to the developmental period of interest (e.g., tassel development or seed maturation) in the plant. Any identifiable promoter which causes the desired temporal and spatial expression may be used in the methods of the present disclosure. Many leaf-preferred promoters are known in the art (Yamamoto et al. (1997) Plant J. 12(2):255-265; Kwon et al. (1994) Plant Physiol. 105:357-367; Yamamoto et al. (1994) Plant Cell Physiol. 35(5):773-778; Gotor et al. (1993) Plant J. 3:509-518; Orozco et al. (1993) Plant Mol. Biol. 23(6):1129-1138; and Matsuoka et al. (1993) Proc. Natl. Acad. Sci. USA 90(20):9586-9590).

Promoters which are seed or embryo-specific and may be useful in the disclosure include soybean Kunitz trypsin inhibitor (Kti3, Jofuku and Goldberg. (1989) Plant Cell 1:1079-1093), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein(barley endosperm) (Marris, C., et al. (1988) Plant Mol. Biol. 10:359-366), glutenin and gliadin (wheat endosperm) (Colot, V., et al. (1987) EMBO J. 6:3559-3564). Promoters of seed-specific genes operably linked to heterologous coding regions in chimeric gene constructions maintain their temporal and spatial expression pattern in transgenic plants. Such examples include Arabidopsis 2S seed storage protein gene promoter to express enkephalin peptides in Arabidopsis and Brassica napus seeds (Vanderkerckhove et al. (1989) Bio/Technology 7: L929-932), bean lectin and bean beta-phaseolin promoters to express luciferase (Riggs et al. (1989) Plant Sci. 63:47-57), and wheat glutenin promoters to express chloramphenicol acetyl transferase (Colot et al. (1987) EMBO J 6:3559-3564).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Also contemplated are synthetic promoters which include a combination of one or more heterologous regulatory elements.

The promoter of the recombinant DNA constructs of the invention can be any type or class of promoter known in the art, such that any one of a number of promoters can be used to express the various polynucleotide sequences disclosed herein, including the native promoter of the polynucleotide sequence of interest. The promoters for use in the recombinant DNA constructs of the invention can be selected based on the desired outcome.

The recombinant DNA constructs of the present disclosure may also include other regulatory elements, including but not limited to, translation leader sequences, introns, and polyadenylation recognition sequences. In certain embodiments, a recombinant DNA construct further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg. (1988) Mol. Cell Biol. 8:4395-4405; Callis et al. (1987) Genes Dev. 1:1183-1200).

### C. Plants and Plant Cells

Provided are plants, plant cells, plant parts, seed and grain comprising in its genome any of the recombinant DNA constructs described herein, so that the plants, plant cells, plant parts, seed, and/or grain have increased expression of the encoded polypeptide.

Also provided are plants, plant cells, plant parts, seeds, and grain comprising an introduced genetic modification at a genomic locus that encodes a polypeptide comprising an amino acid sequence that is at least 80% identical to an amino acid sequence selected from the group consisting of SEQ ID NOs: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158. In certain embodiments, the genetic modification increases the activity of the encoded polypeptide. In certain embodiments, the genetic modification increases the level of the encoded polypeptide. In certain embodiments, the genetic modification increases both the level and activity of the encoded polypeptide.

The plant may be a monocotyledonous or dicotyledonous plant, for example, a rice or maize or soybean plant, such as a maize hybrid plant or a maize inbred plant. The plant may also be sunflower, sorghum, canola, wheat, alfalfa, cotton, barley, millet, sugar cane or switchgrass.

In certain embodiments the plant exhibits increased drought tolerance when compared to a control plant. In certain embodiments, the plant exhibits an alteration of at least one agronomic characteristic when compared to the control plant.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

D. Stacking with Other Traits of Interest

In some embodiments, the inventive polynucleotides disclosed herein are engineered into a molecular stack. Thus, the various host cells, plants, plant cells, plant parts, seeds, and/or grain disclosed herein can further comprise one or more traits of interest. In certain embodiments, the host cell, plant, plant part, plant cell, seed, and/or grain is stacked with any combination of polynucleotide sequences of interest in order to create plants with a desired combination of traits. As used herein, the term “s tacked” refers to having multiple traits present in the same plant or organism of interest. For example, “stacked traits” may comprise a molecular stack where the sequences are physically adjacent to each other. A trait, as used herein, refers to the phenotype derived from a particular sequence or groups of sequences. In one embodiment, the molecular stack comprises at least one polynucleotide that confers tolerance to glyphosate. Polynucleotides that confer glyphosate tolerance are known in the art.

In certain embodiments, the molecular stack comprises at least one polynucleotide that confers tolerance to glyphosate and at least one additional polynucleotide that confers tolerance to a second herbicide.

In certain embodiments, the plant, plant cell, seed, and/or grain having an inventive polynucleotide sequence may be stacked with, for example, one or more sequences that confer tolerance to: an ALS inhibitor; an HPPD inhibitor; 2,4-D; other phenoxy auxin herbicides; aryloxy phenoxypropionate herbicides; dicamba; glufosinate herbicides; herbicides which target the protox enzyme (also referred to as “protox inhibitors”).

The plant, plant cell, plant part, seed, and/or grain having an inventive polynucleotide sequence can also be combined with at least one other trait to produce plants that further comprise a variety of desired trait combinations. For instance, the plant, plant cell, plant part, seed, and/or grain having an inventive polynucleotide sequence may be stacked with polynucleotides encoding polypeptides having pesticidal and/or insecticidal activity, or a plant, plant cell, plant part, seed, and/or grain having an inventive polynucleotide sequence may be combined with a plant disease resistance gene.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

### Methods:

Provided is a method for increasing drought tolerance and/or increasing grain yield, in a plant, comprising increasing the expression of at least one polynucleotide encoding a polypeptide with amino acid sequence of at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158.

In certain embodiments, the method comprises: (a) expressing in a regenerable plant cell a recombinant DNA construct comprising a regulatory element operably linked to the polynucleotide encoding the polypeptide; and (b) generating the plant, wherein the plant comprises in its genome the recombinant DNA construct. In certain embodiments the regulatory element is a heterologous promoter.

In certain embodiments, the method comprises: (a) introducing in a regenerable plant cell a targeted genetic modification at a genomic locus that encodes the polypeptide; and (b) generating the plant, wherein the level and/or activity of the encoded polypeptide is increased in the plant. In certain embodiments the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALE N), engineered site-specific meganucleases, or Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at least 80% identical to an amino acid sequence selected from the group consisting of SEQ ID NOs: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158.

In certain embodiments the DNA modification is an insertion of one or more nucleotides, preferably contiguous, in the genomic locus. For example, the insertion of an expression modulating element (EME), such as an EME described in PCT/US2018/025446, in operable linkage with the gene. In certain embodiments, the targeted DNA modification may be the replacement of the endogenous polypeptide promoter with another promoter known in the art to have higher expression. In certain embodiments, the targeted DNA modification may be the insertion of a promoter known in the art to have higher expression into the 5′UTR so that expression of the endogenous polypeptide is controlled by the inserted promoter. In certain embodiments, the DNA modification is a modification to optimize Kozak context to increase expression. In certain embodiments, the DNA modification is a polynucleotide modification or SNP at a site that regulates the stability of the expressed protein.

The plant for use in the inventive methods can be any plant species described herein. In certain embodiments, the plant is maize, soybean, or rice. Various methods can be used to introduce a sequence of interest into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide disclosed herein, for example, as part of an expression cassette, stably incorporated into their genome.

Transformed plant cells which are derived by plant transformation techniques, including those discussed above, can be cultured to regenerate a whole plant which possesses the transformed genotype (i.e., an inventive polynucleotide), and thus the desired phenotype, such as increased yield. For transformation and regeneration of maize see, Gordon-Kamm et al., The Plant Cell, 2:603-618 (1990).

Various methods can be used to introduce a genetic modification at a genomic locus that encodes a polypeptide disclosed herein into the plant, plant part, plant cell, seed, and/or grain. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In some embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

In addition to modification by a double strand break technology, modification of one or more bases without such double strand break are achieved using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

These fusions contain dCas9 or Cas9 nickase and a suitable deaminase, and they can convert e.g., cytosine to uracil without inducing double-strand break of the target DNA. Uracil is then converted to thymine through DNA replication or repair. Improved base editors that have targeting flexibility and specificity are used to edit endogenous locus to create target variations and improve grain yield. Similarly, adenine base editors enable adenine to inosine change, which is then converted to guanine through repair or replication. Thus, targeted base changes i.e., C⋅G to T⋅A conversion and A⋅T to G⋅C conversion at one more location made using appropriate site-specific base editors.

In an embodiment, base editing is a genome editing method that enables direct conversion of one base pair to another at a target genomic locus without requiring double-stranded DNA breaks (DSBs), homology-directed repair (HDR) processes, or external donor DNA templates. In an embodiment, base editors include (i) a catalytically impaired CRISPR-Cas9 mutant that are mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; and (iv) nickase activity to cleave the non-edited DNA strand, followed by cellular DNA repair processes to replace the G-containing DNA strand.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012).

Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

### EXAMPLES

The following are examples of specific embodiments of some aspects of the invention. The examples are offered for illustrative purposes only and are not intended to limit the scope of the invention in any way.

### Example 1

### Cloning and Vector Construction of Drought Tolerance Genes

A binary construct that contains four multimerized enhancer elements derived from the Cauliflower Mosaic Virus 35S (CaMV 35S) promoter was used, and the rice activation tagging population was developed from four Japonica (Oryza sativa ssp. Japonica) varieties (Zhonghua 11, Chaoyou 1, Taichung 65 and Nipponbare), which were transformed by Agrobacteria-mediated transformation method as described by Lin and Zhang ((2005) Plant Cell Rep. 23:540-547). The transgenic lines generated were developed and the transgenic seeds were harvested to form the rice activation tagging population.

Drought tolerance tagging lines (ATLs) were confirmed in repeated field experiments and their T-DNA insertion loci were determined. The T-DNA insertion loci in the ATLs were determined by Reverse-PCR or Southern-by-Sequencing method (Zastrow-Hayes G. M. et al. (2015), The Plant Genome, 8:1-15). The genes near by the left border and right border of the T-DNA were cloned and the functional genes were recapitulated by field screens. Only the recapitulated functional genes are showed herein. Based on LOC IDs and the corresponding gene sequences of these genes shown in Table 2, primers were designed for cloning the rice drought tolerance genes OsFBX335 (use SEQ ID NOs: 39 and 40), OsNADH1 (use SEQ ID NOs: 41 and 42), OsPUF1 (use SEQ ID NOs: 43 and 44), OsPK1 (use SEQ ID NOs: 45 and 46), OsFAO1 (use SEQ ID NOs: 47 and 48), OsDN-DTP17 (use SEQ ID NOs: 49 and 50), OsCuAO1 (use SEQ ID NOs: 51 and 52), OsDRH7 (use SEQ ID NOs: 53 and 54), OsDN-DTP9 (use SEQ ID NOs: 55 and 56), OsDN-DTP18 (use SEQ ID NOs: 57 and 58), OsSCP52 (use SEQ ID NOs: 59 and 60), and OsDN-DTP19 (use SEQ ID NOs: 61 and 62).

TABLE 2
Rice gene names, Gene IDs (from TIGR) and Construct IDs

| Gene name | LOC ID | Construct ID |
|---|---|---|
| OsFBX335 | LOC_Os09g32860.1 | DP0955 |
| OsNADH1 | LOC_Os07g41300.1 | DP0952 |
| OsPUF1 | LOC_Os03g19700.1 | DP1091 |
| OsPK1 | LOC_Os01g47080.1 | DP1004 |
| OsFAO1 | LOC_Os02g40840.1 | DP2185 |
| OsDN-DTP17 | LOC_Os01g19400.1 | DP0965 |
| OsCuAO1 | LOC_Os04g39120.1 | DP1385 |
| OsDRH7 | LOC_Os09g34910.1 | DP0933 |
| OsDN-DTP9 | LOC_Os07g45810.1 | DP2311 |
| OsDN-DTP18 | LOC_Os09g12780.1 | DP2387 |
| OsSCP52 | LOC_Os11g24320.1 | DP0225 |
| OsDN-DTP19 | LOC_Os01g11360.1 | DP1055 |

PCR amplified products were extracted after the agarose gel electrophoresis using a column kit and then ligated with TA cloning vectors. The sequences and orientation in these constructs were confirmed by sequencing. Each gene was cloned into a plant binary construct.

### Example 2

### Transformation and Gene Expression Analysis of Transgenic Rice Lines

Zhonghua 11 (Oryza sativa L.) were transformed with either a vector prepared in Example 1 or an empty vector (DP0158) by Agrobacteria-mediated transformation as described by Lin and Zhang ((2005) Plant Cell Rep. 23:540-547). Transgenic seedlings (T₀) generated in the transformation laboratory were transplanted in field to get T₁ seeds. The T₁ and subsequent T₂ seeds were screened to confirm transformation and positively identified transgenic seeds were used in the following trait screens.

The gene expression levels in the leaves of the transgenic rice plants were determined by RT-PCR. Primers were designed for the RT-PCR analyses of OsFBX335 (use SEQ ID NOs: 63 and 64), OsDRH7 (use SEQ ID NOs: 65 and 66), and OsPK1 (use SEQ ID NOs: 67 and 68) genes in the over-expression transgenic rice. The level of expression in ZH11-TC (tissue cultured ZH11 rice) was set at 1.00, and the expression levels in the DP0955, DP0933 and DP1004-transgenic rice plants were compared to ZH11-TC. Gene expression was normalized based on the EF-1α mRNA levels, and the results from the gene expression analysis are provided in Table 3 below.

TABLE 3
Relative Expression Level Fold Increase in Transgenic Rice Plants

| Gene name | Construct ID | Relative Expression Level Fold Increase |
|---|---|---|
| OFBX335 | DP0955 | from 97.14 to 93892.60 |
| OsDRH7 | DP0933 | From 2.72 to 324.88 |
| OsPK1 | DP1004 | From 10.23 to 160.62 |

### Example 3

### Characterization of the Transgenic Rice Plants

The transgenic rice plants from Example 2 and ZH11-TC and DP0158 rice plants were tested for drought tolerance.

T₂ seeds from the plants of Example 2 were sterilized by 800 ppm carbendazol for 8 hours at 32° C. and washed 3-5 time, soaked in water for 16 hours at 32° C., and germinated for 18 hours at 35-37° C. in an incubator. Germinated seeds were used as follows for the drought tolerance test.

Drought tolerance assay—The germinated seeds were planted in a seedbed field. At 3-leaf stage, the seedlings were transplanted into the testing field with 4 replicates and 10 plants per replicate for each transgenic line, and the 4 replicates were planted in the same block. ZH11-TC and DP0158 seedlings were nearby the transgenic lines in the same block and were used as controls in the statistical analysis. The rice plants were managed by normal practice using pesticides and fertilizers. Watering was stopped at the panicle initiation stage, so as to give drought stress at flowering stage depending on the weather conditions (temperature and humidity). The soil water content was measured every 4 days at about 10 sites per block using TDR30 (Spectrum Technologies, Inc.). Plant phenotypes were observed and recorded during the experiments. The phenotypes include heading date, leaf rolling degree, drought sensitivity and drought tolerance. Special attention was paid to leaf rolling degree at noontime. At the end of the growing season, six representative plants of each transgenic line were harvested from the middle of the row per line, and grain yield per plant was measured. The grain yield data were statistically analyzed using mixed linear model.

The results from these studies are provided in Table 4, which provides the combined data of the transgenic lines for each of the constructs.

TABLE 4
Agronomic Characteristics of the Transgenic Rice Plants

| | | Avg. yield per plant under field |
|---|---|---|
| No | Construct ID | drought conditions (g/plant) |
| 1 | ZH11-TC | 7.57 ± 0.97 |
| | DP0158 | 6.26 ± 0.97 |
| | DP0955 | 10.51 ± 0.73 ^{a, b} |
| 2 | ZH11-TC | 3.36 ± 0.92 |
| | DP0158 | 3.07 ± 0.91 |
| | DP0952 | 4.63 ± 0.67 ^{a, b} |
| 3 | ZH11-TC | 7.49 ± 1.06 |
| | DP0158 | 6.94 ± 1.06 |
| | DP1091 | 9.80 ± 0.80 ^{a, b} |
| 4 | ZH11-TC | 3.52 ± 0.91 |
| | DP0158 | 2.61 ± 0.91 |
| | DP1004 | 4.70 ± 0.77 ^{a, b} |
| 5 | ZH11-TC | 7.72 ± 0.95 |
| | DP0158 | 9.11 ± 0.95 |
| | DP2185 | 11.51 ± 1.06 ^{a, b} |
| 6 | ZH11-TC | 4.57 ± 0.68 |
| | DP0158 | 4.03 ± 0.68 |
| | DP0965 | 5.98 ± 0.64 ^{a, b} |
| 7 | ZH11-TC | 16.54 ± 2.86 |
| | DP0158 | 16.18 ± 2.84 |
| | DP1385 | 21.58 ± 2.65 ^{a, b} |
| 8 | ZH11-TC | 2.28 ± 0.65 |
| | DP0158 | 2.02 ± 0.65 |
| | DP0933 | 3.57 ± 0.49 ^{a, b} |
| 9 | ZH11-TC | 2.21 ± 0.49 |
| | DP0158 | 2.39 ± 0.36 |
| | DP2311 | 3.23 ± 0.32 ^{a, b} |
| 10 | ZH11-TC | 1.57 ± 0.62 |
| | DP0158 | 1.60 ± 0.62 |
| | DP2387 | 2.81 ± 0.5 ^{a, b} |
| 11 | ZH11-TC | 5.38 ± 0.79 |
| | DP0158 | 5.75 ± 0.79 |
| | DP0225 | 8.01 ± 0.70 ^{a, b} |
| 12 | ZH11-TC | 7.15 ± 0.95 |
| | DP0158 | 6.50 ± 0.95 |
| | DP1055 | 9.50 ± 0.90 ^{a, b} |

^{a}P ≤ 0.1 compared to ZH11-TC control;
^{b}P ≤ 0.1 compared to DP0158 control.

DP0955-transgenic rice plants were tested two times in Hainan field in two years. The results consistently showed that the average yield per plant of DP0955-transgenic rice increased under field drought conditions compared to the controls. As shown in Table 4, twelve lines were tested in Hainan field, 10 of them showed significantly increased (P<0.1) yield as compared to the yield of ZH11-TC and DP0158 controls. The average yield per plant of these 12 lines is 39% and 68% higher than that of ZH11-TC and DP0158 controls, respectively. These data show that OsFBX335 is a rice drought tolerance gene. DP0952-transgenic rice plants were tested two times in Hainan field in two years. The results consistently showed that the average yield per plant of DP0952-transgenic rice increased under field drought conditions compared to the controls. As shown in Table 4, twelve lines were tested; 6 lines showed significantly increased yield (P<0.1) as compared to the yield of the DP0158 control; and 5 lines showed significantly increased yield (P<0.1) as compared to the yield of the ZH11-TC control. The average yield per plant of these 12 lines is 38% and 51% A higher than that of ZH11-TC and DP0158 controls, respectively. These data show that OsNADH1 is a rice drought tolerance gene.

DP1091-transgenic rice plants were tested two times in Hainan field in two years. The results consistently showed that the average yield per plant of DP1091-transgenic rice increased under field drought conditions compared to the controls. As shown in Table 4, twelve lines were tested; 5 lines showed significantly increased yield (P<0.1) as compared to the yield of the DP0158 control; and 4 lines showed significantly increased yield (P<0.1) as compared to the yield of the ZH11-TC control. The average yield per plant of these 12 lines is 31% and 41% higher than that of ZH11-TC and DP0158 controls, respectively. These results demonstrate that OsPUF1 transgenic rice plants had enhanced drought tolerance compared to both controls.

DP1004-transgenic rice plants were tested two times in Hainan and Ningxia fields in two years. The results consistently showed that the average yield per plant of DP1004-transgenic rice increased under field drought conditions compared to the controls. As shown in Table 4, twelve lines were tested, and three lines showed good seed setting rate in Hainan field. Ten lines showed significantly increased yield (P<0.1) as compared to the yield of the DP0158 control and 6 lines showed significantly increased yield (P<0.1) as compared to the yield of the ZH11-TC control. The average yield per plant of these 12 lines is 34% and 80% higher than that of ZH11-TC and DP0158 controls, respectively. These results demonstrate that OsPK1 transgenic rice plants had enhanced drought tolerance compared to both controls.

DP2185-transgenic rice plants were tested two times in Hainan field in two years. The results consistently showed that the average yield per plant of DP2185-transgenic rice increased under field drought conditions compared to the controls. As shown in Table 4, twelve lines were tested, and six lines observed good seed setting rate in Hainan field. Eight lines showed significantly increased yield (P<0.1) as compared to the yield of the DP0158 control and 7 lines showed significantly increased yield (P<0.1) as compared to the yield of the ZH11-TC control. The average yield per plant of these 12 lines is 49% and 26% higher than that of ZH11-TC and DP0158 controls, respectively. These results demonstrate that OsFAO1 transgenic rice plants had enhanced drought tolerance compared to both controls.

DP0965-transgenic rice plants were tested two times in Hainan field in two years. The results consistently showed that the average yield per plant of DP0965-transgenic rice increased under field drought conditions compared to the controls. As shown in Table 4, eight lines were tested, and two lines observed good seed setting rate in Hainan field. Seven lines showed significantly increased yield (P<0.1) as compared to the yield of the DP0158 control and 5 lines showed significantly increased yield (P<0.1) as compared to the yield of the ZH11-TC control. The average yield per plant of these 8 lines is 31% and 48% higher than that of ZH11-TC and DP0158 controls, respectively. These results demonstrate that OsDN-DTP17 transgenic rice plants had enhanced drought tolerance compared to both controls.

DP1385-transgenic rice plants were tested two times in Hainan and Ningxia field in two years. The results consistently showed that the average yield per plant of DP1385-transgenic rice increased under field drought conditions compared to the controls. As shown in Table 4, eight lines were tested, and four lines observed good seed setting rate in Hainan field. Four lines showed significantly increased yield (P<0.1) as compared to the yield of the DP0158 control and 2 lines showed significantly increased yield (P<0.1) as compared to the yield of the ZH11-TC control. The average yield per plant of these 8 lines is 30% and 34% higher than that of ZH11-TC and DP0158 controls, respectively. These results demonstrate that OsCuAO1 transgenic rice plants had enhanced drought tolerance compared to both controls.

DP0933-transgenic rice plants were tested two times in Hainan and Ningxia field in two years. The results consistently showed that the average yield per plant of DP0933-transgenic rice increased under field drought conditions compared to the controls. As shown in Table 4, eight lines were tested, and four lines observed good seed setting rate in Ningxia field. Five lines showed significantly increased yield (P<0.1) as compared to the yield of the DP0158 control and 3 lines showed significantly increased yield (P<0.1) as compared to the yield of the ZH11-TC control. The average yield per plant of these 8 lines is 57% and 77% higher than that of ZH11-TC and DP0158 controls, respectively. These results demonstrate that OsDRH7 transgenic rice plants had enhanced drought tolerance compared to both controls.

DP2311-transgenic rice plants were tested two times in Hainan and Ningxia field in two years. The results consistently showed that the average yield per plant of DP2311-transgenic rice increased under field drought conditions compared to the controls. As shown in Table 4, eight lines were tested, and two lines observed good seed setting rate in Hainan field. Six lines showed significantly increased yield (P<0.1) as compared to the yield of the DP0158 control and 6 lines showed significantly increased yield (P<0.1) as compared to the yield of the ZH11-TC control. The average yield per plant of these 8 lines is 46% and 35% higher than that of ZH11-TC and DP0158 controls, respectively. These results demonstrate that OsDN-DTP9 transgenic rice plants had enhanced drought tolerance compared to both controls.

DP2387-transgenic rice plants were tested two times in Hainan and Ningxia field in two years. The results consistently showed that the average yield per plant of DP2387-transgenic rice increased under field drought conditions compared to the controls. As shown in Table 4, eight lines were tested in Hainan field. Five lines showed significantly increased yield (P<0.1) as compared to the yield of the DP0158 and ZH11-TC controls. The average yield per plant of these 8 lines is 79% and 76% higher than that of ZH11-TC and DP0158 controls, respectively. These results demonstrate that OsDN-DTP18 transgenic rice plants had enhanced drought tolerance compared to both controls.

DP0225-transgenic rice plants were tested two times in Hainan field in two years. The results consistently showed that the average yield per plant of DP0225-transgenic rice increased under field drought conditions compared to the controls. As shown in Table 4, eight lines were tested, and five lines observed good seed setting rate in Hainan field. Four lines showed significantly increased yield (P<0.1) as compared to the yield of the DP0158 and ZH11-TC controls. The average yield per plant of these 8 lines is 49% and 39% higher than that of ZH11-TC and DP0158 controls, respectively. These results demonstrate that OsSCP52 transgenic rice plants had enhanced drought tolerance compared to both controls.

DP1055-transgenic rice plants were tested two times in Hainan and Ningxia field in two years. The results consistently showed that the average yield per plant of DP1055-transgenic rice increased under field drought conditions compared to the controls. As shown in Table 4, twelve lines were tested, and two lines observed good seed setting rate in Hainan field. Eight lines showed significantly increased yield (P<0.1) as compared to the yield of the DP0158 control and 6 lines showed significantly increased yield (P<0.1) as compared to the yield of the ZH11-TC control. The average yield per plant of these 12 lines is 33% and 46% higher than that of ZH11-TC and DP0158 controls, respectively. These results demonstrate that OsDN-DTP19 transgenic rice plants had enhanced drought tolerance compared to both controls.

Taken together, these results indicate that OsFBX335, OsNADH1, OsPUF1, OsPK1, OsFAO1, OsDN-DTP17, OsCuAO1, OsDRH7, OsDN-DTP9, OsDN-DTP18, OsSCP52 and OsDN-DTP19 transgenic rice plants have increased tolerance to drought stresses compared to control plants.

### Example 4

### Transformation and Evaluation of Maize with Rice Drought Tolerance Genes

Maize plants will be transformed with one of the polynucleotides encoding the polypeptides described herein or a corresponding homolog from maize, Arabidopsis, or other species. Expression of the gene in the maize transformation vector can be under control of a constitutive promoter such as the maize ubiquitin promoter (Christensen et al. (1989) Plant Mol. Biol. 12:619-632 and Christensen et al. (1992) Plant Mol. Biol. 18:675-689) or under control of another promoter, such as a stress-responsive promoter or a tissue-preferred promoter. The recombinant DNA construct can be introduced into maize cells by particle bombardment substantially as described in International Patent Publication WO 2009/006276. Alternatively, maize plants can be transformed with the recombinant DNA construct by Agrobacterium-mediated transformation substantially as described by Zhao et al. in Meth. Mol. Biol. 318:315-323 (2006) and in Zhao et al., Mol. Breed. 8:323-333 (2001) and U.S. Pat. No. 5,981,840 issued Nov. 9, 1999.

Progeny of the regenerated plants, such as T₁ plants, can be subjected to a soil-based drought stress. Using image analysis, plant area, volume, growth rate and color can be measured at multiple times before and during drought stress. Significant delay in wilting or leaf area reduction, a reduced yellow-color accumulation, and/or an increased growth rate during drought stress, relative to a control, will be considered evidence that the gene functions in maize to enhance drought tolerance.

### Example 5

### Laboratory Drought Screening of Rice Drought Tolerance Genes in Arabidopsis

To understand whether rice drought tolerance genes can improve dicot plants' drought tolerance, or other traits, the rice expression vectors described herein can be transformed into Arabidopsis (Columbia) using floral dip method by Agrobacterium mediated transformation procedure and transgenic plants were identified (Clough, S. T. and Bent, A. F. (1998) The Plant Journal 16, 735-743; Zhang, X. et al. (2006) Nature Protocols 1:641-646).

Progeny of the regenerated plants, such as T₁ plants, can be subjected to a soil-based drought stress. Using image analysis, plant area, volume, growth rate and color can be measured at multiple times before and during drought stress. Significant delay in wilting or leaf area reduction, a reduced yellow-color accumulation, and/or an increased growth rate during drought stress, relative to a control, will be considered evidence that the gene functions in dicot plants to enhance drought tolerance.

1. (canceled)

2. (canceled)

3. (canceled)

4. (canceled)

5. (canceled)

6. (canceled)

7. A modified plant or modified seed comprising an increased expression of at least one polynucleotide encoding a polypeptide comprising an amino acid sequence of at least 90% sequence identity to SEQ ID NOs: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158, wherein the modified plant or plant grown from the modified seed has improved drought tolerance when compared to a control plant.

8. The modified plant or modified seed of claim 7, wherein the modified plant or modified seed comprises in its genome a recombinant DNA construct comprising a polynucleotide encoding a polypeptide with amino acid sequence of at least 90% sequence identity to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158 operably linked to at least one regulatory element.

9. The modified plant or modified seed of claim 7, wherein the modified plant or modified seed comprises a targeted genetic modification at a genomic locus comprising a polynucleotide sequence encoding a polypeptide with an amino acid sequence of at least 90% sequence identity to SEQ ID NOs: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158, thereby increasing expression of the polypeptide.

10. The modified plant or modified seed of claim 7, wherein said plant is selected from the group consisting of rice, maize, soybean, sunflower, sorghum, canola, wheat, alfalfa, cotton, barley, millet, sugar cane and switchgrass.

11. A method of increasing drought tolerance in a plant, comprising increasing the expression of at least one polynucleotide encoding a polypeptide comprising an amino acid sequence of at least 90% sequence identity to SEQ ID NOs: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158.

12. The method of claim 11, wherein the method comprises:
a) expressing in a regenerable plant cell a recombinant DNA construct comprising a regulatory element operably liked to the polynucleotide sequence; and
b) generating the plant, wherein the plant comprises in its genome the recombinant DNA construct.

13. The method of claim 11, wherein the method comprises:
a) introducing in a regenerable plant cell a targeted genetic modification at a genomic locus that encodes a polypeptide comprising an amino acid sequence of at least 90% sequence identity compared to SEQ ID NOs: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158; and
b) generating the plant, wherein the level and/or activity of the polypeptide is increased in the plant.

14. The method of claim 13, wherein the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute.

15. The method of claim 13, wherein the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at least 90% identical to an amino acid sequence selected from the group consisting of SEQ ID NOs: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158.

16. The method of claim 12, wherein the regulatory element is a heterologous promoter.

17. A method of increasing drought tolerance in a plant, the method comprising:
(a) expressing in a regenerable plant cell, a polynucleotide operably linked to at least one heterologous regulatory element, wherein the polynucleotide encodes a polypeptide having an amino acid sequence of at least 90% sequence identity to SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158 and the expression level of the polynucleotide is increased compared to that of a control plant; and
(b) selecting a plant comprising the polynucleotide operably linked to the heterologous regulatory element for increased drought tolerance as compared to a control plant not comprising the polynucleotide operably linked to the heterologous regulatory element.

18. The method of claim 17, wherein the heterologous regulatory element is a promoter.

19. The method of claim 17 or 18, wherein said plant is selected from the group consisting of rice, maize, soybean, sunflower, sorghum, canola, wheat, alfalfa, cotton, barley, millet, sugar cane and switchgrass.

20. The modified plant or seed of claim 7, wherein the polynucleotide comprises the nucleotide sequence of SEQ ID NO: 1, SEQ ID NO: 2, SEQ ID NO: 4, SEQ ID NO: 6, SEQ ID NO: 7, SEQ ID NO: 9, SEQ ID NO: 10, SEQ ID NO: 12, SEQ ID NO: 13, SEQ ID NO: 15, SEQ ID NO: 16, SEQ ID NO: 18, SEQ ID NO: 19, SEQ ID NO: 21, SEQ ID NO: 22, SEQ ID NO: 24. SEQ ID NO: 25, SEQ ID NO: 27, SEQ ID NO: 28, SEQ ID NO: 30, SEQ ID NO: 31, SEQ ID NO: 33, SEQ ID NO: 34, SEQ ID NO: 36, SEQ ID NO: 37, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157.

21. The modified plant or seed of claim 7, wherein the encoded polypeptide comprises the amino acid sequence of SEQ ID NO: 3, 5, 8, 11, 14, 17, 20, 23, 26, 29, 32, 35, 38, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158.

22. The modified plant or seed of claim 7, wherein the modified plant or plant grown from the seed has increased grain yield under drought conditions.

23. The modified plant or seed of claim 8, wherein the regulatory element is a heterologous promoter.


SEQUENCE LISTING (158 sequences)

SEQ ID NO: 1 - DNA, 3085 bp, Oryza sativa
ggcgcgaattccggcgaggtattcgccgcgttgccatggggaagatgctggatccgaagcgtctcatgtctgcgcgacaggagcggcatcgccgtcggcggcgacggcaactccgaccccgcagtagccctccctctcctaccttcttcttcttcagcaattgtgtttgatagccacgattgcttttttggggaaaaaaaagagcatctcgcttgcaagcaattcaagaaggggcaatgggacattatgatctgtggaatttaggaacaaaatagtgtagaattgtttttttcttttgggaggtaaaaggtagcaatgctcggttgaatttgatttgtaggaagtaatgaaacaaattggagtgaatacccgctcttttcacaaaaagctggacaactgaaccaaattgaattgttcgtgttcattcatgtgctttgatctgatgttttcccctgttgattgatgtctgcatataaccacgcaaatgatgtgtttgttcaattgcttcactggctaaaaggaagggctcattctgtcaccacgttgcagattctgaaggcgccaaaagaacaagatattcagtgcctagccttccagaggtatggtacctaatgctgataagaaatacttatattagaagcaaagtgggtcactatgattctatgactgctctaagtagtctctaactacacactggtggtcatatggtggctagatcaaaaggattgagatcctctaccttcaaactcaaaggtaccatgtctttgtcactgacagatgggtccatggttcattgggcctacatgttagtgacaaaggcacgtgcctttgaagttagaggagcctgatccagatttatgaacagatatttatagctgcaatcttctacaattctgcatgagggcaccttttctctgttctagctgttgcacctttatcactcaagcagaaaattgtttgtgatggcgtcaccttaataatcttgttaaggcagttcactttctaaaggttcttcctcacttatcagcgggttcctgtacttcttttatgtgattactctggtctcgaccagtttcctgatgatcaatgtttatgtttaccagaacccccaatgtgcgatttgtccttaagatacatgtttatttttttcttcattacctttgactaatttataggatgatattgtagggagaaaagcatcatttcatacataacattgttgactaccgctgcatacttcaagcaagaataggtttcatactatgtgatcataattagtcactggctacaatctgctttatctttttcccgatagccactaagaaaactttgaaagggaaaaggagaaaaatgaaaatgtgccgttcagtttgcgtaccacaaatcccaaagagctattccttataagctactaatagtactgctttcccttccaattgaaccttcacaattcttttaaaaaaacttttgtccttgctagtgcttgtaggaaatctggtgccatatacactccctaatgtcattcaaagatgctgctcgtgcagcatgcgtgtctcgtgcttttcgtcattcctggcgatgccatcccaatcttatatgttgtattggaatactgggctcggatttcatcaacaaatttgaccgcattatgaaaaatcactcaggaattggcatcaagtcggtaaagtttcaatataatagtttttacaatactagaaggagcactagcatcagccatcatttcgatagttggcatcagattgctattacaccgtggatcgaagaactcaccattagcctatctctatcttcattcaatatggagtacagcttcccatgctcacttctagctgatgggagagcaagctcaatgcggcatctttatcttggcaattgtggattccatcccacaatcaaccttgacttgagaaacctgacaaggttgcatctgattaatgtacatattacaggagatgagttagggtgccttctctcaaattctcattgtttggagcagttagaacttatgtattgcaatggaataatttgtctgaagataccttccctgttgcagtgcctcagccacctagaagtgtttgattgcagaattcaagtagttgagaataaggctccaaatctttgcagttttgactttggaggtaggcaggtacaactcttacttggcgaatcgttgcgaatgaagagactatccttgcactaccccgatgctatgtattatgctcatgcaaagcttccatccaatgcgccaaatcttgaaactctcaccctctgatgctcgactgacaaggtactcaaaaaacataaatgatcatagacttgtgatgtccatataacaatattcatgccttcagcctgatgatctttatgctttttatgcagatggttgatacaccaatattgcctagcaaattcctccatctcaagtgcttgaccattgatcataccattatctcatcttccccagcttataattatctttacctggtttattttctcgacgcttgcccttccttggagactttccttttgggtgtaagtcaattttaatcctgcaaacttatgattaagtgactagcccatcaaatgaaaaagtttattattggttttcatgtccagatatcgcagaaccacatggagcatgactcgattattggagattcctcacatatgaggcaaatgccaggacaccgtcatgacaacctccggagtgtggagatcaccggtttctactctgcaaagagcttgatcgagctaacatgttatattcttgacaatacagttgcactcaagtaccttacactggatacaactcgtggttttttcagctgttctacaggtgaacacgacagatgctttcctatggacaaaatcatgataacggaagccaaaagggcagtcctggctatccaaacatacatcgagcgaaaagttccttccacggtcaagctaaatgttgtgaagccttgcagccgttgccatgctgttgaatcatatttgtaattattatttttcttcaacttaattttcttggtaattcggtgattttaaagtgtccggtgctttc

SEQ ID NO: 2 - DNA, 1236 bp, Oryza sativa
atggggaagatgctggatccgaagcgtctcatgtctgcgcgacaggagcggcatcgccgtcggcggcgacggcaactccgaccccgcaattctgaaggcgccaaaagaacaagatattcagtgcctagccttccagaggaaatctggtgccatatacactccctaatgtcattcaaagatgctgctcgtgcagcatgcgtgtctcgtgcttttcgtcattcctggcgatgccatcccaatcttatatgttgtattggaatactgggctcggatttcatcaacaaatttgaccgcattatgaaaaatcactcaggaattggcatcaagtcggtaaagtttcaatataatagtttttacaatactagaaggagcactagcatcagccatcatttcgatagttggcatcagattgctattacaccgtggatcgaagaactcaccattagcctatctctatcttcattcaatatggagtacagcttcccatgctcacttctagctgatgggagagcaagctcaatgcggcatctttatcttggcaattgtggattccatcccacaatcaaccttgacttgagaaacctgacaaggttgcatctgattaattgcctcagccacctagaagtgtttgattgcagaattcaagtagttgagaataaggctccaaatctttgcagttttgactttggaggtaggcagatggttgatacaccaatattgcctagcaaattcctccatctcaagtgcttgaccattgatcataccattatctcatcttccccagcttataattatctttacctggtttattttctcgacgcttgcccttccttggagactttccttttgggtatatcgcagaaccacatggagcatgactcgattattggagattcctcacatatgaggcaaatgccaggacaccgtcatgacaacctccggagtgtggagatcaccggtttctactctgcaaagagcttgatcgagctaacatgttatattcttgacaatacagttgcactcaagtaccttacactggatacaactcgtggttttttcagctgttctacaggtgaacacgacagatgctttcctatggacaaaatcatgataacggaagccaaaagggcagtcctggctatccaaacatacatcgagcgaaaagttccttccacggtcaagctaaatgttgtgaagccttgcagccgttgccatgctgttgaatcatatttgtaa

SEQ ID NO: 3 - protein, 411 aa, Oryza sativa
MGKMLDPKRLMSARQERHRRRRRRQLRPRNSEGAKRTRYSVPSLPEEIWCHIHSLMSFKDAARAACVSRAFRHSWRCHPNLICCIGILGSDFINKFDRIMKNHSGIGIKSVKFQYNSFYNTRRSTSISHHFDSWHQIAITPWIEELTISLSLSSFNMEYSFPCSLLADGRASSMRHLYLGNCGFHPTINLDLRNLTRLHLINCLSHLEVFDCRIQVVENKAPNLCSFDFGGRQMVDTPILPSKFLHLKCLTIDHTIISSSPAYNYLYLVYFLDACPSLETFLLGISQNHMEHDSIIGDSSHMRQMPGHRHDNLRSVEITGFYSAKSLIELTCYILDNTVALKYLTLDTTRGFFSCSTGEHDRCFPMDKIMITEAKRAVLAIQTYIERKVPSTVKLNVVKPCSRCHAVESYL

SEQ ID NO: 4 - DNA, 1071 bp, Oryza sativa
atgtcattcaaagatgctgctcgtgcagcatgcgtgtctcgtgcttttcgtcattcctggcgatgccatcccaatcttatatgttgtattggaatactgggctcggatttcatcaacaaatttgaccgcattatgaaaaatcactcaggaattggcatcaagtcggtaaagtttcaatataatagtttttacaatactagaaggagcactagcatcagccatcatttcgatagttggcatcagattgctattacaccgtggatcgaagaactcaccattagcctatctctatcttcattcaatatggagtacagcttcccatgctcacttctagctgatgggagagcaagctcaatgcggcatctttatcttggcaattgtggattccatcccacaatcaaccttgacttgagaaacctgacaaggttgcatctgattaattgcctcagccacctagaagtgtttgattgcagaattcaagtagttgagaataaggctccaaatctttgcagttttgactttggaggtaggcagatggttgatacaccaatattgcctagcaaattcctccatctcaagtgcttgaccattgatcataccattatctcatcttccccagcttataattatctttacctggtttattttctcgacgcttgcccttccttggagactttccttttgggtatatcgcagaaccacatggagcatgactcgattattggagattcctcacatatgaggcaaatgccaggacaccgtcatgacaacctccggagtgtggagatcaccggtttctactctgcaaagagcttgatcgagctaacatgttatattcttgacaatacagttgcactcaagtaccttacactggatacaactcgtggttttttcagctgttctacaggtgaacacgacagatgctttcctatggacaaaatcatgataacggaagccaaaagggcagtcctggctatccaaacatacatcgagcgaaaagttccttccacggtcaagctaaatgttgtgaagccttgcagccgttgccatgctgttgaatcatatttgtaa

SEQ ID NO: 5 - protein, 356 aa, Oryza sativa
MSFKDAARAACVSRAFRHSWRCHPNLICCIGILGSDFINKFDRIMKNHSGIGIKSVKFQYNSFYNTRRSTSISHHFDSWHQIAITPWIEELTISLSLSSFNMEYSFPCSLLADGRASSMRHLYLGNCGFHPTINLDLRNLTRLHLINCLSHLEVFDCRIQVVENKAPNLCSFDFGGRQMVDTPILPSKFLHLKCLTIDHTIISSSPAYNYLYLVYFLDACPSLETFLLGISQNHMEHDSIIGDSSHMRQMPGHRHDNLRSVEITGFYSAKSLIELTCYILDNTVALKYLTLDTTRGFFSCSTGEHDRCFPMDKIMITEAKRAVLAIQTYIERKVPSTVKLNVVKPCSRCHAVESYL

SEQ ID NO: 6 - DNA, 1174 bp, Oryza sativa
ccatgttacgagcttctggaatacaatgggatcttcgtaaagttgatctttacgagtcttacaatcaattcgattggaaaatccaatggcaaaaagaaggggattcattagcacgctatttagtacgaatcggtgaaacgagggaatcaatcaaaattattcaacaggctgtagaaaaaattcctgggagcccttatgagaatttagaagtccgacgctttaagaaagcaaagaattccaaatggaatgattttgaatatcgatttcttggtaaaaaaccttcacccaattttgaattgtcaaaacaagagtggaagccccaaaaggtgaattaggaatttatctggtaggagatgatagtcttttcccctggagatggaaaattcgtccacccggttttattaatttgcaaattcttcctcagcaagtcaaaaaaatgaattggctgatatcagcgggcattctaggccatgacctagagcctaagatttaagggttaattggttccataccactgcaactttaccaaattaaaaaaataccactactatccatcatatcgctgctgggtgccactgcaattttagaaaattggatccatgccactccgtcgcattttccatccactcctgttgatgccgtctctccgcgtctaaggaccgagccagaggacaccaccaccgacttcggcttcggcaacgacaccgacgcaggcgcgagcagcggaggcgactcagggttctcgtggtggcgcaggcgcgcaagtggcggccggaatcccgtgcggagcggcccccatgccacgacggcggttgaggatgccgcaggggagcgatgcagctgctcctcgtcgcctcgccggtcattcgccgctgcaaggacctcgtcgcgcgtgccctcggccagggacgccgacgccaccggccggagacggcggcgcagccgcggcccactctgccgcctcctccttggatgcctcggcttcctcgtcgcatgccccgacaacacgatggtcgccgagtcgacgcccgtgaggtggcttccgctgtcagtgtcgtcaacggcgaggctgacctgggtggcggcgaaggagctcgaccgcggtgagggagctcggcggtggcggcgagggagctcggcaccggagaccgcaccagagagaccccactaggcc

SEQ ID NO: 7 - DNA, 852 bp, Oryza sativa
atgttacgagcttctggaatacaatgggatcttcgtaaagttgatctttacgagtcttacaatcaattcgattggaaaatccaatggcaaaaagaaggggattcattagcacgctatttagtacgaatcggtgaaacgagggaatcaatcaaaattattcaacaggctgtagaaaaaattcctgggagcccttatgagaatttagaagtccgacgctttaagaaagcaaagaattccaaatggaatgattttgaatatcgatttcttgaaaattggatccatgccactccgtcgcattttccatccactcctgttgatgccgtctctccgcgtctaaggaccgagccagaggacaccaccaccgacttcggcttcggcaacgacaccgacgcaggcgcgagcagcggaggcgactcagggttctcgtggtggcgcaggcgcgcaagtggcggccggaatcccgtgcggagcggcccccatgccacgacggcggttgaggatgccgcaggggagcgatgcagctgctcctcgtcgcctcgccggtcattcgccgctgcaaggacctcgtcgcgcgtgccctcggccagggacgccgacgccaccggccggagacggcggcgcagccgcggcccactctgccgcctcctccttggatgcctcggcttcctcgtcgcatgccccgacaacacgatggtcgccgagtcgacgcccgtgaggtggcttccgctgtcagtgtcgtcaacggcgaggctgacctgggtggcggcgaaggagctcgaccgcggtgagggagctcggcggtggcggcgagggagctcggcaccggagaccgcaccagagagaccccactag

SEQ ID NO: 8 - protein, 283 aa, Oryza sativa
MLRASGIQWDLRKVDLYESYNQFDWKIQWQKEGDSLARYLVRIGETRESIKIIQQAVEKIPGSPYENLEVRRFKKAKNSKWNDFEYRFLENWIHATPSHFPSTPVDAVSPRLRTEPEDTTTDFGFGNDTDAGASSGGDSGFSWWRRRASGGRNPVRSGPHATTAVEDAAGERCSCSSSPRRSFAAARTSSRVPSARDADATGRRRRRSRGPLCRLLLGCLGFLVACPDNTMVAESTPVRWLPLSVSSTARLTWVAAKELDRGEGARRWRRGSSAPETAPERPH

SEQ ID NO: 9 - DNA, 1333 bp, Oryza sativa
cttcccgggcctcttcaaaaatccccgcgccgcgtagccgatcacttgtcgcaccaacatggaggaggacggcggcagcgacgcgcgggtggtggagtccatgcagcggctgctcgacgcggtgccgcccggtgccgacgacccgtacaccatcttccgcctccccgcggccgtgcgggagcggcaccgcgacctgtacgagccaaagctcgtgtccgtggggccgtactaccacggccgcgacgggctcggcgccgcgcagcgccacaagtggcgcctcctccgcgacttcctgtcgcggcagagcgacgacaaggccgggctcggagcctacgtgcgcgccgcgcgggcggtcgaggccgacgcgcggcagtgctacgcggaggggttcgacgacgtgggcgccgacgagttcgccgagatgctggtgctcgacggttgcttcctgctcgagttcttcctgaggaagagcgaggggcagctcgcggcgcccgggggcgccaagtgggcgtggcagcacatgtaccacgacgtcctcctcctggagaatcagataccgttcttcgtcgtcgagaggctgcacggcgtcgcctttgccggggacgatgatggcgccgccgaccgtgacgcgctcctcgacatcttctgcaaggcgttcgccggcgacctgccttcgagccgcgtcatacggccgccgagcgacaagaccatccaccacctgctgcacctgcactacgagtgcaacgtccgcaacccggccgcggacagcgacaaggcgcgcaacggcggggacgccgccaacggcggcgcgtcgtcgctggccatctggaagcagccgcccgtcccgtccccgcgctccagcgacggcgccatcaaagggcggatgacgtcgatgatcccgccggcggccaagatggaggaggccggcgtgacgttcaagcggaaggccaccccgcgcgacgtgttcgacatgagcttccggtacggcgtgctgcacatgccggcgttcgtggtggacgagggcgccaaggtgctcctcgcgaacctggtggcgttcgagcagggcggcggccgcgcggcgcggaagctggaagggggcaacctggcgacggggttcgtggcgctgctgggttcgctggtgaactcgcggcgggacgtggaggtgctccgccgctgcgggatcctgcactgcatggtcaccgacgaggaggccgtggcgtacttcagccacgtggtgcagtacacgaccatggactacgaccggcacctgctggcttgcctgttccgggacatccgggagcactgccactggaaccgatgatttgggtgtttc

SEQ ID NO: 10 - DNA, 1263 bp, Oryza sativa
atggaggaggacggcggcagcgacgcgcgggtggtggagtccatgcagcggctgctcgacgcggtgccgcccggtgccgacgacccgtacaccatcttccgcctccccgcggccgtgcgggagcggcaccgcgacctgtacgagccaaagctcgtgtccgtggggccgtactaccacggccgcgacgggctcggcgccgcgcagcgccacaagtggcgcctcctccgcgacttcctgtcgcggcagagcgacgacaaggccgggctcggagcctacgtgcgcgccgcgcgggcggtcgaggccgacgcgcggcagtgctacgcggaggggttcgacgacgtgggcgccgacgagttcgccgagatgctggtgctcgacggttgcttcctgctcgagttcttcctgaggaagagcgaggggcagctcgcggcgcccgggggcgccaagtgggcgtggcagcacatgtaccacgacgtcctcctcctggagaatcagataccgttcttcgtcgtcgagaggctgcacggcgtcgcctttgccggggacgatgatggcgccgccgaccgtgacgcgctcctcgacatcttctgcaaggcgttcgccggcgacctgccttcgagccgcgtcatacggccgccgagcgacaagaccatccaccacctgctgcacctgcactacgagtgcaacgtccgcaacccggccgcggacagcgacaaggcgcgcaacggcggggacgccgccaacggcggcgcgtcgtcgctggccatctggaagcagccgcccgtcccgtccccgcgctccagcgacggcgccatcaaagggcggatgacgtcgatgatcccgccggcggccaagatggaggaggccggcgtgacgttcaagcggaaggccaccccgcgcgacgtgttcgacatgagcttccggtacggcgtgctgcacatgccggcgttcgtggtggacgagggcgccaaggtgctcctcgcgaacctggtggcgttcgagcagggcggcggccgcgcggcgcggaagctggaagggggcaacctggcgacggggttcgtggcgctgctgggttcgctggtgaactcgcggcgggacgtggaggtgctccgccgctgcgggatcctgcactgcatggtcaccgacgaggaggccgtggcgtacttcagccacgtggtgcagtacacgaccatggactacgaccggcacctgctggcttgcctgttccgggacatccgggagcactgccactggaaccgatga

SEQ ID NO: 11 - protein, 420 aa, Oryza sativa
MEEDGGSDARVVESMQRLLDAVPPGADDPYTIFRLPAAVRERHRDLYEPKLVSVGPYYHGRDGLGAAQRHKWRLLRDFLSRQSDDKAGLGAYVRAARAVEADARQCYAEGFDDVGADEFAEMLVLDGCFLLEFFLRKSEGQLAAPGGAKWAWQHMYHDVLLLENQIPFFVVERLHGVAFAGDDDGAADRDALLDIFCKAFAGDLPSSRVIRPPSDKTIHHLLHLHYECNVRNPAADSDKARNGGDAANGGASSLAIWKQPPVPSPRSSDGAIKGRMTSMIPPAAKMEEAGVTFKRKATPRDVFDMSFRYGVLHMPAFVVDEGAKVLLANLVAFEQGGGRAARKLEGGNLATGFVALLGSLVNSRRDVEVLRRCGILHCMVTDEEAVAYFSHVVQYTTMDYDRHLLACLFRDIREHCHWNR

SEQ ID NO: 12 - DNA, 1796 bp, Oryza sativa
ctcgctctccattcccaaatctcctccgcatcgggatggcgcaggtggtggctgcggcagggacggcggcggcggtggcggcggtggggaggccgctgggtggcggcggatctggggccgacgcgctccgcccggcggcgaggctgtcgttcgcgccgcgctggtgcggtgggagcgcgggggcagcaagggcgcgccgcgagtccgccgtcacgtccgtgattagccgcgccccgcgtctcgacgccgaggtgctgccggtgtcggccgacgacgacgcggacgtgaaggaagaagaaaacttccagcatctcaaggccattcagcaacttgcaacatcagccaatggtgtgtggtctaaaccaaatgtgagacgcaagacaaagatcgtgtgcaccattggtccttcaactaacacaaaggagatgatatggaaacttgctgaggctggcatgaatgtggctcggcttaatatgtcacatggagaccatgcatcacaccagaaagttattgacttagtaaaggaatataatgctcaaacaaaggataacgtgattgctattatgcttgacacgaagggaccagaagttaggagtggagatttgcctcaaccaataatgttggaaactggccaggagttcactttcacaattaagagaggagttgggactgagacatgtgttagcgttaactatgatgactttgttaatgatgtagaagtgggtgacatgctcctcgtagatggtggaatgatgtcgttgttggtcaagtccaaaactgaagattcggtaaaatgtgaagttattgatggaggtgaactgaaatccaggcgtcacctgaatgttcgtggaaagagcgcaaccttgccatcaataactgacaaggattgggatgatattaagtttggtgtggagaatcaagttgactactatgccgtttcttttgtcaaagatgctcaagttgtgcatgaattgaaggattatctcagaagctccaatgccgatatacatgtaattgtaaagattgaaagtgcagattccattccaaacctacactcaatcatcacagcatcggatggggctatggttgccagaggtgacttgggagctgaactccctattgaggaggtaccgctgctgcaggaagaaattattagaatgtgcaggagcatgggaaaagctgttattgttgccactaatatgctagaaagtatgattgttcatccaactccaacccgagcagaagtttcagacattgctatagctgttcgagaaggttctgatggcattatgctttctggggaaactgcacacggaaagtttcctttgaaagctgttaaggtcatgcatactgttgccctgagaactgaagccactatgtctggtggggaaacaccggctaaccttggtcaggtgttcaagaatcacatgagtgaaatgtttgcataccattcaacaatgatgtccaacactcttggaacatcaattgtggttttcaccaggacaggatttatggctatcctacttagtcattaccgcccttctggcaccatttttgcctttacggaccaggagagagttagacaacgattggctttgtaccaaggtgtatgtccagtccagatggaattttctgatgatgctgagaagacatttggtgacgctttgtcatacttgctgaaacatggtatggtgaaggaaggcgaggaggttgcacttgttcaaagtggcaggcaacctatctggcggtcgcagtccacccataatattcaggtcaggaaggtttgatgtgaagac

SEQ ID NO: 13 - DNA, 1752 bp, Oryza sativa
atggcgcaggtggtggctgcggcagggacggcggcggcggtggcggcggtggggaggccgctgggtggcggcggatctggggccgacgcgctccgcccggcggcgaggctgtcgttcgcgccgcgctggtgcggtgggagcgcgggggcagcaagggcgcgccgcgagtccgccgtcacgtccgtgattagccgcgccccgcgtctcgacgccgaggtgctgccggtgtcggccgacgacgacgcggacgtgaaggaagaagaaaacttccagcatctcaaggccattcagcaacttgcaacatcagccaatggtgtgtggtctaaaccaaatgtgagacgcaagacaaagatcgtgtgcaccattggtccttcaactaacacaaaggagatgatatggaaacttgctgaggctggcatgaatgtggctcggcttaatatgtcacatggagaccatgcatcacaccagaaagttattgacttagtaaaggaatataatgctcaaacaaaggataacgtgattgctattatgcttgacacgaagggaccagaagttaggagtggagatttgcctcaaccaataatgttggaaactggccaggagttcactttcacaattaagagaggagttgggactgagacatgtgttagcgttaactatgatgactttgttaatgatgtagaagtgggtgacatgctcctcgtagatggtggaatgatgtcgttgttggtcaagtccaaaactgaagattcggtaaaatgtgaagttattgatggaggtgaactgaaatccaggcgtcacctgaatgttcgtggaaagagcgcaaccttgccatcaataactgacaaggattgggatgatattaagtttggtgtggagaatcaagttgactactatgccgtttcttttgtcaaagatgctcaagttgtgcatgaattgaaggattatctcagaagctccaatgccgatatacatgtaattgtaaagattgaaagtgcagattccattccaaacctacactcaatcatcacagcatcggatggggctatggttgccagaggtgacttgggagctgaactccctattgaggaggtaccgctgctgcaggaagaaattattagaatgtgcaggagcatgggaaaagctgttattgttgccactaatatgctagaaagtatgattgttcatccaactccaacccgagcagaagtttcagacattgctatagctgttcgagaaggttctgatggcattatgctttctggggaaactgcacacggaaagtttcctttgaaagctgttaaggtcatgcatactgttgccctgagaactgaagccactatgtctggtggggaaacaccggctaaccttggtcaggtgttcaagaatcacatgagtgaaatgtttgcataccattcaacaatgatgtccaacactcttggaacatcaattgtggttttcaccaggacaggatttatggctatcctacttagtcattaccgcccttctggcaccatttttgcctttacggaccaggagagagttagacaacgattggctttgtaccaaggtgtatgtccagtccagatggaattttctgatgatgctgagaagacatttggtgacgctttgtcatacttgctgaaacatggtatggtgaaggaaggcgaggaggttgcacttgttcaaagtggcaggcaacctatctggcggtcgcagtccacccataatattcaggtcaggaaggtttga

SEQ ID NO: 14 - protein, 583 aa, Oryza sativa
MAQVVAAAGTAAAVAAVGRPLGGGGSGADALRPAARLSFAPRWCGGSAGAARARRESAVTSVISRAPRLDAEVLPVSADDDADVKEEENFQHLKAIQQLATSANGVWSKPNVRRKTKIVCTIGPSTNTKEMIWKLAEAGMNVARLNMSHGDHASHQKVIDLVKEYNAQTKDNVIAIMLDTKGPEVRSGDLPQPIMLETGQEFTFTIKRGVGTETCVSVNYDDFVNDVEVGDMLLVDGGMMSLLVKSKTEDSVKCEVIDGGELKSRRHLNVRGKSATLPSITDKDWDDIKFGVENQVDYYAVSFVKDAQVVHELKDYLRSSNADIHVIVKIESADSIPNLHSIITASDGAMVARGDLGAELPIEEVPLLQEEIIRMCRSMGKAVIVATNMLESMIVHPTPTRAEVSDIAIAVREGSDGIMLSGETAHGKFPLKAVKVMHTVALRTEATMSGGETPANLGQVFKNHMSEMFAYHSTMMSNTLGTSIVVFTRTGFMAILLSHYRPSGTIFAFTDQERVRQRLALYQGVCPVQMEFSDDAEKTFGDALSYLLKHGMVKEGEEVALVQSGRQPIWRSQSTHNIQVRKV

SEQ ID NO: 15 - DNA, 932 bp, Oryza sativa
gaagacgacgactcactgctagctggtagctgctgccggtggtgccgatcgactgccggacggcgggcagagacgccatgggagaggagaagagccagcagcggcggcggcagggccaccctctcctgaggggaggcggcgcggggaagcaggccgggcggcgttacacccacggcttctccgcctcccagatggtcgcgctcgccgccctgtgcggcgcgctggcgccctcgctcccgccggacacccgcgacgacgacgacgacgacgccggaggcggacgctatggcggcgccggtgcgtccgacgccaaggccgtcagggacttcctcctcgcctccgccgccgacccgcccgtccccgacgaggttggtactacaactcggcctacgcgcttaacaagcttgcttttgcttagctacctgtagcgagagtgaagcctcggccggcgatggtgcaggtggcggagctgatgacgaggatgtgcctccgggaggcgctggcgctggtgcgggcggtgctgtggctgctggggacgcggctggggacgctggcgctgtgcggggggcggtgcgtgtcgtgggggaggtggccgttcgtgctcacgttcgcggagatgccggtggagcggcgggaggaggcgctcgggcggtggagcagggtgaccgtgctcccgccgctgcgcgccttcttcctcgtcgtcaaggtcttctgcctctacgtcttctactcctgggtaaccactcctcctcctcctcctctcatggcctctcgtacaccgctgatcgatcagctgcgagttcattcacgttagatttttttttctcgcgttttgcccgcgccacgcacgtgcgcgcggtgatttttctctcgacggatgcacgtacgtacgcccgtgagggacggactaagcgttgtggggactgtttatg

SEQ ID NO: 16 - DNA, 645 bp, Oryza sativa
atgggagaggagaagagccagcagcggcggcggcagggccaccctctcctgaggggaggcggcgcggggaagcaggccgggcggcgttacacccacggcttctccgcctcccagatggtcgcgctcgccgccctgtgcggcgcgctggcgccctcgctcccgccggacacccgcgacgacgacgacgacgacgccggaggcggacgctatggcggcgccggtgcgtccgacgccaaggccgtcagggacttcctcctcgcctccgccgccgacccgcccgtccccgacgaggtggcggagctgatgacgaggatgtgcctccgggaggcgctggcgctggtgcgggcggtgctgtggctgctggggacgcggctggggacgctggcgctgtgcggggggcggtgcgtgtcgtgggggaggtggccgttcgtgctcacgttcgcggagatgccggtggagcggcgggaggaggcgctcgggcggtggagcagggtgaccgtgctcccgccgctgcgcgccttcttcctcgtcgtcaaggtcttctgcctctacgtcttctactcctgggtaaccactcctcctcctcctcctctcatggcctctcgtacaccgctgatcgatcagctgcgagttcattcacgttag

SEQ ID NO: 17 - protein, 214 aa, Oryza sativa
MGEEKSQQRRRQGHPLLRGGGAGKQAGRRYTHGFSASQMVALAALCGALAPSLPPDTRDDDDDDAGGGRYGGAGASDAKAVRDFLLASAADPPVPDEVAELMTRMCLREALALVRAVLWLLGTRLGTLALCGGRCVSWGRWPFVLTFAEMPVERREEALGRWSRVTVLPPLRAFFLVVKVFCLYVFYSWVTTPPPPPLMASRTPLIDQLRVHSR

SEQ ID NO: 18 - DNA, 2416 bp, Oryza sativa
gtctctctccagcttctctctcccccatggctctcgcgtgcgcatcgcgccgcctcctcgccggcgccggaactccggcgagatccttccactcccagccctaccaaggtaaccccctctcgtctcctcctcctcctccgcctcctccctcttggttattgtgattggagctcgattgctgcgggcgcagccaaggtcggcgtggtggagttcctcaacggggtcgggaagggggtggaaacgcacgccgcgaaggtggaggaggcggtgggaggcgacctccagaggctcctcgagacccgcacgctgcggctcaagaagctcggcatcccctgcaagcatgtacgtcctatacctgtctgtccctggtatgcctttggtagtagagtctctggttcatgtgcagatttgagcagcggcttgatgttgttgccgattatgttagtgagtgggggtattcagcatttcgtaatgtgatggtatgtttttggttagattttgctgtataatgttgccattgttggatgatgtcgttgcgtttgtttggtagtagtgaaccgagtaactgatttctgtgtgcttaaaactatggctcgttgcattatgcgtagatttgtttgaaaaatcatacgtactgttgattttagagtaaatttcacaataccacatgcttttggctccaagttgcacaaaaccctgggggttttagccctacgcacttaacaattataagtgttgctttttggggagagttcttggtattgaatcggattggattgcaatgtattccttggtcaatagattggacaaaatgtattttggcgtgtgcttggaggttaacattgttggagagtaatttggtaaatgtgctatctaactcagtgttttgcttttcctgcttatttgtctctaggctgtgcatttctggatagacatttgacatgaaaaatctgaagttctgtttttatttttttctaaatagttgttcttgtgaataataaagctatgtccgtgcaaagatttttcaaaagaatataccattgctttgaggataggtatatgttgtcagttttcagaatccaattagcaaaagctaagtagttctgccatcgtgtcgtgctttgaagggaacttacctctttgacctgagatgttgatatgtatcttttattattggtagcaactggtagtgaattactcaatactcaatagagagttgtggtattctgtagcatgttcttcactgggtgatcacatttttacttgattaagccataaaattggcatttttatgctttttagatgtcttggtgaatttggcaattatactgtgactttagaaatttgaagtgcaaaatacacatttttttttccttcaagacaattgtagtacttttgttttaagaccaaaggccagaatagccctgattttataaaccagatggtaaatacagaggtacaattgtaatatttggatgcaaataaagttctatggatttgcatttctacttatggtggatctttggtcgatgcaaagtgaacacacatgcagcttatgacctgcatgctaaaggaaatatccatttctttttgttaggtaagggtaaactgatcccattttcacagagaaaatgatggcataggctaaaggtttggagatgtgtcgtacttgttcttaccgtatttatctccctcctgatttctgttcttactgtttctttttcttcttttcttcctagcttcaccaatttgtgtttgtctagtgaagaataatcttggctgtctgttgttaatcacatggttaggaaatttattatgtttccataatccgtcccttttttaataccttaaaggcaatcccattgaatgatttgctgccagtatattgagacatgtgaagtcttcgtcagtagattatgatgttttctaagaaatagacaccattgtaggtttgtgggtgacatttagctcctcctaacctataacacaaacatatttcagattctccaaatctccagtaaaactgggaccattgcatttgcaaccacaaaccagctaaagtatcaaggtgaccaagccactaaattgtatactaaggttgctcacccacccaagcacttaaaaggcagggtggccactcgagttaatcgatacataaaattgattgtgggatctggctggcggaacctcttcctaaaacttatctttatttatcaactatgttcacttttttctgttatctttgtgttgagatatgggaaaataatttctagggaatatattaatttaaaattattttctccctatgatgcagaggaagttaattttgagttttgctcacaagtatcgtcttggtctttggaagccccaagcagaagcgaagaaagcgcaatgaagttgagttgtcttgaagtgg

SEQ ID NO: 19 - DNA, 315 bp, Oryza sativa
atggctctcgcgtgcgcatcgcgccgcctcctcgccggcgccggaactccggcgagatccttccactcccagccctaccaagccaaggtcggcgtggtggagttcctcaacggggtcgggaagggggtggaaacgcacgccgcgaaggtggaggaggcggtgggaggcgacctccagaggctcctcgagacccgcacgctgcggctcaagaagctcggcatcccctgcaagcataggaagttaattttgagttttgctcacaagtatcgtcttggtctttggaagccccaagcagaagcgaagaaagcgcaatga

SEQ ID NO: 20 - protein, 104 aa, Oryza sativa
MALACASRRLLAGAGTPARSFHSQPYQAKVGVVEFLNGVGKGVETHAAKVEEAVGGDLQRLLETRTLRLKKLGIPCKHRKLILSFAHKYRLGLWKPQAEAKKAQ

SEQ ID NO: 21 - DNA, 710 bp, Oryza sativa
tccacgagcaatttcactacaacgatggcggtatcttgatcaagttagactagtgtttggtttatcgccatagttataacatgccatattttcttggcaaaatttgctacagaacacgcaaaaaatatgtaattagctgggagacaccaaaaaacgtgacatggttgatggccactacaaaatttgtgtaattggctatagaacactggtcgaattattttattatttccgagttccatcgttttctccttcgtcaggaatgcgcgtggtggggcgaggtcggcgtgagcggggtgagcgcgagctcaacggcagcagtagggcgggtgaggaagcactggctcgcgctgaggagcggggcgaacctattcatgcactcgctgaagctcgatgagccggaaaagtccgtcgtcaagagctggcggaagggcgccaacccactcccgcctcgctgcaccgtggtcgtcatccggttccgtggcaagtcccgagtcctcggcgagggcaacggtgtggtgactcctctgcatgtccctgcctccggttacccgatgatgagcatggacgagcagaccagcctcagcttcgcgctattcaaagacgtggcgttcaacgccagcatccgccgcggcgtcagggcctccgacgtcgcttgcctgcccatctccttcggctagtagggcccagtggaggaaaactggtgactcatc

SEQ ID NO: 22 - DNA, 426 bp, Oryza sativa
atggcggaatgcgcgtggtggggcgaggtcggcgtgagcggggtgagcgcgagctcaacggcagcagtagggcgggtgaggaagcactggctcgcgctgaggagcggggcgaacctattcatgcactcgctgaagctcgatgagccggaaaagtccgtcgtcaagagctggcggaagggcgccaacccactcccgcctcgctgcaccgtggtcgtcatccggttccgtggcaagtcccgagtcctcggcgagggcaacggtgtggtgactcctctgcatgtccctgcctccggttacccgatgatgagcatggacgagcagaccagcctcagcttcgcgctattcaaagacgtggcgttcaacgccagcatccgccgcggcgtcagggcctccgacgtcgcttgcctgcccatctccttcggctag

SEQ ID NO: 23 - protein, 141 aa, Oryza sativa
MAECAWWGEVGVSGVSASSTAAVGRVRKHWLALRSGANLFMHSLKLDEPEKSVVKSWRKGANPLPPRCTVVVIRFRGKSRVLGEGNGVVTPLHVPASGYPMMSMDEQTSLSFALFKDVAFNASIRRGVRASDVACLPISFG

SEQ ID NO: 24 - DNA, 2102 bp, Oryza sativa
cttctccgacgatgccttccctccccgtcgccgccgccgagccgatggccgtcgacgagtcggcctccaagaagtcgaagcgcaagctgaaggcggcggaggtggaggtggaggcctcgtcgaggaagaaggagaagaaggagaagaagcggaaggcgaaggagccgtcgccatcgtcctcctcctcatccgaggaggaggagaggagcagcacgagctccgacgagccggcgcccgccgcgaagaaggcgaagaaggagaagacgaaggagaaggtggtggtggaggaggaggaggaggacgacgacgaaggggagctcaccgccagcggcgacgaggatcccgccgaccccaacgcgctcgccaacttcaggatctcggagtcgctcagggagaagctcaagtccaaggggatcaaggcgctgttccccatccaggccaccaccttcgatctcgtcctcgacggccacgacctggtcggccgcgcgcgcactggacagggaaaaacattggcttttgttctaccgatacttgagtcattagttaatgggacacacaaggcatccagaaggactgattatggcaggcctccaactgttttggttctgttgcctaccagagagctagccaagcaggtgcacacagactttgcattttatggtgcaacatttgggctatctgcatgctgcgtatatgggggttctgattatcgttctcaagaaatggcaatcagaaagggggttgacattgttgttggaactcctggtcgtgtgaaggactttgttgaaaagggaaccctcaattttaggtctttgaaattccgtgtccttgatgaggctgatgagatgcttaatatgggcttcgttgatgatgttgagcttattcttggcaaagttgaagatgttacaaaagtacagacacttctcttcagcgctactataccagagtgggtgaagaagctctccttgagatttctcaaatctggaaagaaaacggttgatcttgttggagatgagaaattgaaagctagtgcatctgttaggcatcttgctcttccttgtaaccgtgctgcaagggctcaagttattccagacatcatccgatgctatagccgtggaggccggaccattatctttaccgagacaaaagaatctgcatcagacctctctggtttgattgctggatcacgtgccttgcatggtgatgttgctcaagctcagcgtgaagttattcttgctgggttcaggagtggcaagtttttagttttggttgctacaaacgtggctgcacgagggctggacattaatgatgtgcagcttatcattcagtgtgaacctccacgtgatgttgaagcctacatacaccggtcaggtcggactgggagagcaggcaatactggtgttgctgtcatgctttttgaacccagatataagttcaatgtgaacagaatagaaagggagtctggggtcaaatttgaacatatctctgcaccacagccaactgatgtggcacaatctgctggtactgaagcagcagaagccatttccagtgtttcagacagtgtcattcctgtttttcgggagcaagctgagcaactattgaactcttccgggatgtctgcagttgatctactagccaaagcacttgcgaaggcagttggctacacggacataaagaagagatccttgctgtcttctatggacaaccacactacattacttcttcaaactggtcgatcggtgtatgcagcaggatttgttctttctactttgaaaaggtttatgccagaagagagacttgcagatgtaaagggtataaccatcactgctgatgggacaggtgctgtatttgatgttccttcagcagaggttgaagactatattcaaggtgcacagaacgctgccatggtgactgtcgaggaagtgaaacaattgccacccttgcaagagagggaacagtctggtggcagccgaggtggaggaaggtttggaaacaggagattttctggtggtggtggtggccggggtggaggcggcagagggttcggtggaggcagaggcaggggaggtggcggcggcaacaggtttaacaagaggtactag

SEQ ID NO: 25 - DNA, 2091 bp, Oryza sativa
atgccttccctccccgtcgccgccgccgagccgatggccgtcgacgagtcggcctccaagaagtcgaagcgcaagctgaaggcggcggaggtggaggtggaggcctcgtcgaggaagaaggagaagaaggagaagaagcggaaggcgaaggagccgtcgccatcgtcctcctcctcatccgaggaggaggagaggagcagcacgagctccgacgagccggcgcccgccgcgaagaaggcgaagaaggagaagacgaaggagaaggtggtggtggaggaggaggaggaggacgacgacgaaggggagctcaccgccagcggcgacgaggatcccgccgaccccaacgcgctcgccaacttcaggatctcggagtcgctcagggagaagctcaagtccaaggggatcaaggcgctgttccccatccaggccaccaccttcgatctcgtcctcgacggccacgacctggtcggccgcgcgcgcactggacagggaaaaacattggcttttgttctaccgatacttgagtcattagttaatgggacacacaaggcatccagaaggactgattatggcaggcctccaactgttttggttctgttgcctaccagagagctagccaagcaggtgcacacagactttgcattttatggtgcaacatttgggctatctgcatgctgcgtatatgggggttctgattatcgttctcaagaaatggcaatcagaaagggggttgacattgttgttggaactcctggtcgtgtgaaggactttgttgaaaagggaaccctcaattttaggtctttgaaattccgtgtccttgatgaggctgatgagatgcttaatatgggcttcgttgatgatgttgagcttattcttggcaaagttgaagatgttacaaaagtacagacacttctcttcagcgctactataccagagtgggtgaagaagctctccttgagatttctcaaatctggaaagaaaacggttgatcttgttggagatgagaaattgaaagctagtgcatctgttaggcatcttgctcttccttgtaaccgtgctgcaagggctcaagttattccagacatcatccgatgctatagccgtggaggccggaccattatctttaccgagacaaaagaatctgcatcagacctctctggtttgattgctggatcacgtgccttgcatggtgatgttgctcaagctcagcgtgaagttattcttgctgggttcaggagtggcaagtttttagttttggttgctacaaacgtggctgcacgagggctggacattaatgatgtgcagcttatcattcagtgtgaacctccacgtgatgttgaagcctacatacaccggtcaggtcggactgggagagcaggcaatactggtgttgctgtcatgctttttgaacccagatataagttcaatgtgaacagaatagaaagggagtctggggtcaaatttgaacatatctctgcaccacagccaactgatgtggcacaatctgctggtactgaagcagcagaagccatttccagtgtttcagacagtgtcattcctgtttttcgggagcaagctgagcaactattgaactcttccgggatgtctgcagttgatctactagccaaagcacttgcgaaggcagttggctacacggacataaagaagagatccttgctgtcttctatggacaaccacactacattacttcttcaaactggtcgatcggtgtatgcagcaggatttgttctttctactttgaaaaggtttatgccagaagagagacttgcagatgtaaagggtataaccatcactgctgatgggacaggtgctgtatttgatgttccttcagcagaggttgaagactatattcaaggtgcacagaacgctgccatggtgactgtcgaggaagtgaaacaattgccacccttgcaagagagggaacagtctggtggcagccgaggtggaggaaggtttggaaacaggagattttctggtggtggtggtggccggggtggaggcggcagagggttcggtggaggcagaggcaggggaggtggcggcggcaacaggtttaacaagaggtactag

SEQ ID NO: 26 - protein, 696 aa, Oryza sativa
MPSLPVAAAEPMAVDESASKKSKRKLKAAEVEVEASSRKKEKKEKKRKAKEPSPSSSSSSEEEERSSTSSDEPAPAAKKAKKEKTKEKVVVEEEEEDDDEGELTASGDEDPADPNALANFRISESLREKLKSKGIKALFPIQATTFDLVLDGHDLVGRARTGQGKTLAFVLPILESLVNGTHKASRRTDYGRPPTVLVLLPTRELAKQVHTDFAFYGATFGLSACCVYGGSDYRSQEMAIRKGVDIVVGTPGRVKDFVEKGTLNFRSLKFRVLDEADEMLNMGFVDDVELILGKVEDVTKVQTLLFSATIPEWVKKLSLRFLKSGKKTVDLVGDEKLKASASVRHLALPCNRAARAQVIPDIIRCYSRGGRTIIFTETKESASDLSGLIAGSRALHGDVAQAQREVILAGFRSGKFLVLVATNVAARGLDINDVQLIIQCEPPRDVEAYIHRSGRTGRAGNTGVAVMLFEPRYKFNVNRIERESGVKFEHISAPQPTDVAQSAGTEAAEAISSVSDSVIPVFREQAEQLLNSSGMSAVDLLAKALAKAVGYTDIKKRSLLSSMDNHTTLLLQTGRSVYAAGFVLSTLKRFMPEERLADVKGITITADGTGAVFDVPSAEVEDYIQGAQNAAMVTVEEVKQLPPLQEREQSGGSRGGGRFGNRRFSGGGGGRGGGGRGFGGGRGRGGGGGNRFNKRY

SEQ ID NO: 27 - DNA, 1759 bp, Oryza sativa
atgccgaagatggtggtggaggtggccgaccggtacacggcgaggagggacgggcggccgaacaactccggcgaggcggacggagaggttcgcggcgacgttccggcgaaaccgagggcaggccggggtggagggcgatgcggcgtcgccgagggaggagacggcgacgtcggccgacgctctggcgcggcgaccggggcagccggaggagatgtcgacgaggaggaaggtgaggccggcgcgggcgacggcgttccggcgaaattcgggcgaggaggaggtgatgccggggaagaaggcgatgccgcggtgtcgaaggaggagacggcggcgtcgatcggcgcaccggcgatgaacagcagccggctggagggagtacagtggcaccaatgccactggttgccggcgggggcaccgttccggcgagtttccggcgaggagacggtaaggccggtgaaggggacggtgttgcagagccgagggaggcgacggcacggccggacgatgctccggtgaggagggagaggtggctggaggccggccggcgtcggagagggagaaggagaaggcggggagaggtgatacgaccacggggaggctcgggaaggggctaaaacgatagaacgaaggatgaggatcccattttataggatgggggagagagccggccacgggaggagcgggaacggcgacgggaatggcggccggcggcaaaggaaggtggccggacttggcgcggccgttcccggcgattggaggcaccattcaaggggaaattaagggggaattgaagaggagggaaaggggattaattccccctaattaattttggaattaaccgagtgaatcaagcggatttgagggaggattggcgctagggttcgcacggggagagggaagccgggcggcggctggaggaagaaggcgacggcggcctgacgcgtgggccccacgcggggcgcgcggtcagcgcgcgcacgtgcgcgctcgggcgggagggcgccgctcgagttgggccgggcggccggcccaggaggggggggggaggggtaggcggcttgggctgggcggctgggccgaggcaggccgggaggaaggagggggggaagggagctgggccgagaggggaggaggcccaagaggagggagagaaggagagggagagaaagagagggaggagggaaggacttcgggctgggcttggcccaaaggaggaagaggagggatttttagtttttctttttaataaactttgatcaaggttgttgttgcttaataattatttccggtgctctgaaaattcaagtaaaatttgagggctctttttagaccaaggagaatttaacaaaaattctccgggccacattggaatttttcttgcacgtattttagtgtttgccaatttcttttcgaattttaattaattctattattccttttagcgaatgaattttatttcgggatgaatttatcaggacgtgacaaatccaccccccttacaagaatctcgtccccgagattcgaggaggctagcaagaagataggttgggcggtcttctcttcttgtctgacttcttccgctccgtcttctataacaaacttgcctagcaaaacttctccactctggactgcttggctcaatctcctgttgcagatggaatcttctcttcacctgacttttccctggttgtacctctagttgacttcgactggattatggcacttgaagatgggactttcgttccagaacttctcataa

SEQ ID NO: 28 - DNA, 504 bp, Oryza sativa
atgccgaagatggtggtggaggtggccgaccggtacacggcgaggagggacgggcggccgaacaactccggcgaggcggacggagaggttcgcggcgacgttccggcgaaaccgagggcaggccggggtggagggcgatgcggcgtcgccgagggaggagacggcgacgtcggccgacgctctggcgcggcgaccggggcagccggaggagatgtcgacgaggaggaaggtgaggccggcgcgggcgacggcgttccggcgaaattcgggcgaggaggaggtgatgccggggaagaaggcgatgccgcggtgtcgaaggaggagacggcggcgtcgatcggcgcaccggcgatgaacagcagccggctggagggagtacagtggcaccaatgccactggttgccggcgggggcaccgttccggcgagtttccggcgaggagacgttgacttcgactggattatggcacttgaagatgggactttcgttccagaacttctcataa

SEQ ID NO: 29 - protein, 167 aa, Oryza sativa
MPKMVVEVADRYTARRDGRPNNSGEADGEVRGDVPAKPRAGRGGGRCGVAEGGDGDVGRRSGAATGAAGGDVDEEEGEAGAGDGVPAKFGRGGGDAGEEGDAAVSKEETAASIGAPAMNSSRLEGVQWHQCHWLPAGAPFRRVSGEETLTSTGLWHLKMGLSFQNFS

SEQ ID NO: 30 - DNA, 428 bp, Oryza sativa
ggcgggcgatgcagatccgcgtgcggtgcgggtgcggcgaggcggggtgcccggagtgggccatcgtggaggtgcagggcgtggtgcagccacagccgtgcttctccggccgcatccagggcctccacatcggccgcctctgcgccgccgccgccccgtcttccaaggcggcgttcaccttcaccgtggggtaccatgagctcgccggcaccaaggtggcgctcaagaagcctctcctcgtgctgaggaagaagaagacgacggcggtggcggcggagacggagctggaggtgatcggcgtgatccggcacaagatcctcttcaaggaccggcccaaggctctcatctcaaaaccgcaggtgaaggagaagaagactctgccgctaccggcgccggcggcggcgccaccaccccaatcatcgtagcac

SEQ ID NO: 31 - DNA, 417 bp, Oryza sativa
atgcagatccgcgtgcggtgcgggtgcggcgaggcggggtgcccggagtgggccatcgtggaggtgcagggcgtggtgcagccacagccgtgcttctccggccgcatccagggcctccacatcggccgcctctgcgccgccgccgccccgtcttccaaggcggcgttcaccttcaccgtggggtaccatgagctcgccggcaccaaggtggcgctcaagaagcctctcctcgtgctgaggaagaagaagacgacggcggtggcggcggagacggagctggaggtgatcggcgtgatccggcacaagatcctcttcaaggaccggcccaaggctctcatctcaaaaccgcaggtgaaggagaagaagactctgccgctaccggcgccggcggcggcgccaccaccccaatcatcgtag

SEQ ID NO: 32 - protein, 138 aa, Oryza sativa
MQIRVRCGCGEAGCPEWAIVEVQGVVQPQPCFSGRIQGLHIGRLCAAAAPSSKAAFTFTVGYHELAGTKVALKKPLLVLRKKKTTAVAAETELEVIGVIRHKILFKDRPKALISKPQVKEKKTLPLPAPAAAPPPQSS

SEQ ID NO: 33 - DNA, 3764 bp, Oryza sativa
catccgtgccgagcgagtccaatggcctctaacctcaaccgacctgatttgacatcttggacctggcgttcgatggatgaggtgacaccgggttcgatggggaaggaggagacgctggccccgggagggagctcgggggaggacgccgctgcatcctctattcctctgtgagagctcgagctcgggaggtgaagccggggaggaagtggggtcgcaaggatggaagcgggatcaaggggtcatcattaggagggcggaggcggagggggtggcgtggtttctgccgctgttttgatgctgcgggagtcggtatgtgcggatgcggatggatagtagagaattaatctgtgatttggcctgtaggattgctacagatgaactcctcctttttatattagtataggaatataactagagatgattcggcgattatgctttgaatcatgattttttttatagatattacgggatctattcagaacactcttttgtgagaagttcgaaccatccacttaggaattatactacaatacaaacacactgtggtttgaatttttttcaaaactgcagattagtagtgaactcatcattgtcttcaaataaccaagcttgtgtccattgaattagcataatttgcctctcttaataaaacagtagggatcaataaaataaagcagtggtcaaagttgcattttggtactgttaatgtcaaaatcgatatgttcactagaccagagtagtataaattctgccattatgtctttacgtggaacactttgagatctgtatgcttagtgtattaatgattttgcacatatctatctaataaattatctgcatgaccattgcagctagctagccagctatgcatgtcctctaccatgactagagagaccggttggcaaaagggcataatgcactccagtgttctaaactgtttctttatcatatttctgcattaataaaaaaataacatttcggtgcgcagcggtgaccatgataccgttttgccatttcttggtacacaaacctgggtgaggtcactcaactatcccattgtagatgactggagagcttggcatgtggatggccagtccgcagggtgagttttaattatgtttgattagttctgttgtttggcttacataatcaaaatggttgtctaataagttttcagtatacaaactgctttcaggaataacttttctaaaacttcaattaatttgcagattcaccgtagcatatggaaacaacctgacatttgctactgttaaggtaacaacccatcaatgcacatccaattttatgaataatttatatatagatatttccaaaggttgttgtgaatggagttacgttctgattcacgattacatatcgttgatacaaattcaacccgcgtcatgatgaggatttttaatctgtattactgctaatgatatttactttagttcaaagattattctattttatcaaaaatttcttatattatttaaaaatgaaatcattggtatgcgaagaaatcctcacaacatgggaaagaccttgccacaccagtgttagcaacatttgtggccccaaggttcattttttaaatataagttcattgaaggttcattttttaatttatattagaaaggttaaaagataataaaatttcaaaaactctttagaaggaccttgatatcaccaaggggtcaaattggcgttcctaaacattcgatttgaatccggaacatttggtctaagattaagagttgtgatcttatagcaaaacgtccaatggcaaggtggtaaatcaagaactaaccacttgcagatcaaacaaacaacaaatatagctaccaaaatgtgtgaaacaagatacaaagaaaataacaatatcatcaactagattaaactaagcacataggtagatgaggcaaatgaagaataataatggaaaaacacacgagagacaaaatcaaaatatttgttctgtgatgtttagatccactgattccatttcaaaggccatgtctctttccactcttatactcactaaggcaacaccaaagtgcttctgatgtttagtctttatttcttctattatggaggcaaaatcgaaatcttgacaaacatccatgccacactacataagcttgggtactcaccagcaacacccagccatctaaaagaccttaatgtccaatatagtaatacacacattacaaaatttcctataaactcaagtgctcagacgatgaagtaaactcactagcttacaacatttcaatctgtcactaatttgataaaaaaaagaggggaggggggggatatgtaagaactatatggtcatggatatgcataactctttgtacaaactcaacaactcctaaaggagggggatgagagctttaaatagcatcgttatatttctacccgttaaggtaaaaacaataggtggctgaattaaaataaccataacatctcgctctcaagtctgttttcgaaatcttgaactctatgtaaagcttaattcgacatctaggtaacacaactaaaaccacacaaagtagatgttaaaactgttgattctcttaagatagcacttggatatgtcactttgagcaccgtcaataatcaaggtacaattctgcattcctcttgacaggacataattcctaaactcaaatgcaattagaaaaataactataccaatgagaacacatgtactgtcatggttatggataccaaatacctaatagtagttgattaggcacggctggacccaccacatatcaagtcttatacggaattaggcctcacttacggaaggtattccggataaggaaggacaagtaaagttctatatggaaactataaggactacttggattgtatccatattggtctccctagttctagttggataaggggacacctataggcataaatagaccccctaggaggagagggagataagccacaagacatacaaatcgacacacaagccaggacaaaccaatacgccaccagatatcgacatcagagataagcctagacagaccccatttggtgcctgtagaggccaactaaagggatctaatgccatctttgatctcgatgagttcatattcgaggaggaagactaccctatcgtcgactacgtgttagtgatccatgccgccaagtcgatgacagctagataggttttcccaaatattgtacttgtgtgattcagataaataagagcaacatcgacttcggccaacaggagtagggctattacctgtcagataaggggtccgaacctatataaaatccttgttttcatctcttttacctcaatctcgcatataccctggtaccaacgatccccataccatccaaataccctagccgagacctaaaaggtcgacatgtacgtcatcttctttccttatcatgaggattgccttgatgaattatcactttcgattttagctagatattcatcaatatatgaagtttgaccacttctatatcttcatgccacttgatgtcattcaacaagtaaagtccaatctttacccattttgcagtcaaactcgatgtgggtccacctatgaattacatcttattgatcttcaccatttcatcttgcattcttttaatggttgatgtgggttactcatagattcaactgacctcgggtggaccatcacggcaaagccttcaatcatccttatgaatgaaatggtcacggaaattacctactgacctcgtttctgaccattaccgtt

SEQ ID NO: 34 - DNA, 486 bp, Oryza sativa
atggcctctaacctcaaccgacctgatttgacatcttggacctggcgttcgatggatgaggtgacaccgggttcgatggggaaggaggagacgctggccccgggagggagctcgggggaggacgccgctgcatcctctattcctctcggtgaccatgataccgttttgccatttcttggtacacaaacctgggtgaggtcactcaactatcccattgtagatgactggagagcttggcatgtggatggccagtccgcaggattcaccgtagcatatggaaacaacctgacatttgctactgttaaggtaacacaactaaaaccacacaaagtagatgttaaaactgttgattctcttaagatagcacttggatatgtcactttgagcaccgtcaataatcaagattcaactgacctcgggtggaccatcacggcaaagccttcaatcatccttatgaatgaaatggtcacggaaattacctactga

SEQ ID NO: 35 - protein, 161 aa, Oryza sativa
MASNLNRPDLTSWTWRSMDEVTPGSMGKEETLAPGGSSGEDAAASSIPLGDHDTVLPFLGTQTWVRSLNYPIVDDWRAWHVDGQSAGFTVAYGNNLTFATVKVTQLKPHKVDVKTVDSLKIALGYVTLSTVNNQDSTDLGWTITAKPSIILMNEMVTEITY

SEQ ID NO: 36 - DNA, 2729 bp, Oryza sativa
ctctcatgctcggcgctttctctcccacgtgtctcttcctctcccccagccagatctggtcctgccgctgccaaagctgaccggcgtttgcgcggtagccaggacggtggcggaggggaagtgggaggcagaggtgcggcgatggcggccagaggcagccggtcctgcgcctggccgtgtgggttggggatggctggaggtggtcaggagggcagcgggaaccggcaaggagtcggcgatggagtccctccggacggcggtagggacagcgaagcacggcggaagatggtgacggcgctcggcggctacggcagccctaggggctgcggcgtcccaagacagtggcagtctgagcactgtgaatccgataaagtggaagctgatcccatcggttcgtgtatggttggctagtttcagctaaacgacgaatgacgacgcatgaaagtgggctaactggcggcatgtggcgtcagcttatctgaaatggataaagatggcaccaacggaggtttacattgacggcgcactaggggcggcggcgactagacgactttcactcgtcaaatcgggctgtttcaggagaacaaggtgggacgtagagaaacaaggatgtgtcgaggttgtatggttttcttttcgttttttggttgtgtgttttcctccttgttgaggtgtgagtctaaatactcacgtatcattttggttgtgtatattattcgtggatatagaggccagattaataaaaatttattataaaaaaaatcctaacccaaactgccgaaaacggctttagggtgtgtttgaggagaaggggattgaggagattgggaagataagcaaaacgaggtgagccattaactcatgattaattgagtattaactattttaaatttaaaaaatggattaatatgatttttttaaagcaactttcatatagaaaatttttgcaaaaaacacaccgtttagtagtttgggaagcgtgcgcgtggaaaacgatgtgcattctcactccctatcatccaaacgaacgcagccttagagcatctccaacaggatggctatttttggctctccatttgtctatttagccaactaaccataaagattcctctcccaatctgcgcaaagactctctaaatcctaatggctagtttcctctcaaaaaatccatatattgcatttagttcacacataccatatccatatattgcattggtccatacattgcattgcattcagttcactaaattaaaggtccatacattgcattgcattcagttcactaaattaaaggttcatacattgcattacaaaagttcactaatttaaaagtccatacactacactgcatacattgcattgcaatcagtccagtaatttaaaggtccatacattctattacaaaagtcaaataatttaaaggtccatgcattgcattgcaattagtccactgatttaaaggtccatcatattgcactgcatttagtccgtagcagtacacaaaagtttagaggtgattaattttgatgcaaataaagataccccacttggaatccttaaaccgcttaccaaggatttcagcttgcaatgagtcatagtacattttccttggtccagtcaagcaaaataatgcagttcatacaaaatcaatattgtgagcaccatgcttctccacgcacttgcaaacgcaaacactataacaacaaggtacatttgatgcacacatatttctttcgatcttcagagttgcacacattacagagcaaatacacattacagttgcacacatatttctttcgatctttcgatctacagagcaaatacagaggggattttcgatctacagagccaatacagaggggaaaggagtggtggatgggggttttacctgctttgctatgggcatcggcagggtggtctcagcagggacggcggcggcctcagcaggtcctatcggcagcagtcaatggcggcttcggcaacaaaagtgaaaagacgacggcggaggggcggccctggtagcagaagacgacgacggaggggtggcgcagaggggcggcgctggcggcaaaagacaacggcggtggtcgacggcgtcttggcaacaaaacacaacggcagaggggcgcccctggccgagaggacgccggcggaggggtggcgcacaggggcggcaccggcggagagatgcgcgcgctggatctgggggaagggaaaaacgcgatctgtggaggagaggataggtgggagtagtacacgtgcgtgggcccacgaatggctaggcctttttggctggccaaatttggccagtgcgagcagcactttgtccagccggatggcttggcttggccagtttgttggaggacaaattttaattaaaattgtcaaaatttaatttagagagtagactaaggaggctgttggagatgctcttagtagtagtatcgaacatcgggacataccaaacgagagggattttggtcaaggatttactaaagtagtagtagatcatacgcacacgtatataaacagatccatgaatccatatccatgcatgcatacatgcaccgtcatcaggatattccaactcttttggagacagggtacgtgacagggcccgaaagcgatgtgcctgcccttttttaggctccggccggaactactagcgtagccatgcaccctcgtaaccgagcgacgagatatatcgatcgcgatgtggatggtgccgg

SEQ ID NO: 37 - DNA, 483 bp, Oryza sativa
atggcggccagaggcagccggtcctgcgcctggccgtgtgggttggggatggctggaggtggtcaggagggcagcgggaaccggcaaggagtcggcgatggagtccctccggacggcggtagggacagcgaagcacggcggaagatggtgacggcgctcggcggctacggcagccctaggggctgcggcgtcccaagacagtggcagtctgagcactgtgaatccgataaagtggaagctgatcccatcgagcaaatacagaggggattttcgatctacagagccaatacagaggggaaaggagtggtggatgggggttttacctgctttgctatgggcatcggcagggtggtctcagcagggacggcggcggcctcagcagagagtagactaaggaggctgttggagatgctcttagtagtagtatcgaacatcgggacataccaaacgagagggattttggtcaaggatttactaaagtag

SEQ ID NO: 38 - protein, 160 aa, Oryza sativa
MAARGSRSCAWPCGLGMAGGGQEGSGNRQGVGDGVPPDGGRDSEARRKMVTALGGYGSPRGCGVPRQWQSEHCESDKVEADPIEQIQRGFSIYRANTEGKGVVDGGFTCFAMGIGRVVSAGTAAASAESRLRRLLEMLLVVVSNIGTYQTRGILVKDLLK

SEQ ID NO: 39 - DNA, 24 bp, Artificial Sequence
  Forward primer for cloning gDNA of OsFBX335 gene
ggcgcgaattccggcgaggtattc

SEQ ID NO: 40 - DNA, 26 bp, Artificial Sequence
  Reverse primer for cloning gDNA of OsFBX335 gene
gaaagcaccggacactttaaaatcac

SEQ ID NO: 41 - DNA, 24 bp, Artificial Sequence
  Forward primer for cloning gDNA of OsNADH1 gene
ccatgttacgagcttctggaatac

SEQ ID NO: 42 - DNA, 23 bp, Artificial Sequence
  Reverse primer for cloning gDNA of OsNADH1 gene
ggcctagtggggtctctctggtg

SEQ ID NO: 43 - DNA, 33 bp, Artificial Sequence
  Forward primer for cloning cDNA of OsPUF1 gene
ctgctgaggcttcccgggcctcttcaaaaatcc

SEQ ID NO: 44 - DNA, 36 bp, Artificial Sequence
  Reverse primer for cloning cDNA of OsPUF1 gene
ccgctgagggaaacacccaaatcatcggttccagtg

SEQ ID NO: 45 - DNA, 35 bp, Artificial Sequence
  Forward primer for cloning cDNA of OsPK1 gene
ctgctgaggctcgctctccattcccaaatctcctc

SEQ ID NO: 46 - DNA, 36 bp, Artificial Sequence
  Reverse primer for cloning cDNA of OsPK1 gene
ccgctgagggtcttcacatcaaaccttcctgacctg

SEQ ID NO: 47 - DNA, 38 bp, Artificial Sequence
  Forward primer for cloning gDNA of OsFAO1 gene
ctgctgagggaagacgacgactcactgctagctggtag

SEQ ID NO: 48 - DNA, 36 bp, Artificial Sequence
  Reverse primer for cloning gDNA of OsFAO1 gene
ccgctgaggcataaacagtccccacaacgcttagtc

SEQ ID NO: 49 - DNA, 34 bp, Artificial Sequence
  Forward primer for cloning gDNA of OsDN-DTP17 gene
ctgctgagggtctctctccagcttctctctcccc

SEQ ID NO: 50 - DNA, 36 bp, Artificial Sequence
  Reverse primer for cloning gDNA of OsDN-DTP17 gene
ccgctgaggccacttcaagacaactcaacttcattg

SEQ ID NO: 51 - DNA, 35 bp, Artificial Sequence
  Forward primer for cloning gDNA of OsCuAO1 gene
ctgctgaggtccacgagcaatttcactacaacgat

SEQ ID NO: 52 - DNA, 34 bp, Artificial Sequence
  Reverse primer for cloning gDNA of OsCuAO1 gene
ccgctgagggatgagtcaccagttttcctccact

SEQ ID NO: 53 - DNA, 23 bp, Artificial Sequence
  Forward primer for cloning cDNA of OsDRH7 gene
cttctccgacgatgccttccctc

SEQ ID NO: 54 - DNA, 29 bp, Artificial Sequence
  Reverse primer for cloning cDNA of OsDRH7 gene
ctagtacctcttgttaaacctgttgccgc

SEQ ID NO: 55 - DNA, 30 bp, Artificial Sequence
  Forward primer for cloning gDNA of OsDN-DTP9 gene
ctgctgaggatgccgaagatggtggtggag

SEQ ID NO: 56 - DNA, 35 bp, Artificial Sequence
  Reverse primer for cloning gDNA of OsDN-DTP9 gene
ccgctgaggttatgagaagttctggaacgaaagtc

SEQ ID NO: 57 - DNA, 27 bp, Artificial Sequence
  Forward primer for cloning cDNA of OsDN-DTP18 gene
ctgctgaggggcgggcgatgcagatcc

SEQ ID NO: 58 - DNA, 29 bp, Artificial Sequence
  Reverse primer for cloning cDNA of OsDN-DTP18 gene
ccgctgagggtgctacgatgattggggtg

SEQ ID NO: 59 - DNA, 24 bp, Artificial Sequence
  Forward primer for cloning gDNA of OsSCP52 gene
catccgtgccgagcgagtccaatg

SEQ ID NO: 60 - DNA, 27 bp, Artificial Sequence
  Reverse primer for cloning gDNA of OsSCP52 gene
aacggtaatggtcagaaacgaggtcag

SEQ ID NO: 61 - DNA, 33 bp, Artificial Sequence
  Forward primer for cloning gDNA of OsDN-DTP19 gene
ctgctgaggctctcatgctcggcgctttctctc

SEQ ID NO: 62 - DNA, 32 bp, Artificial Sequence
  Reverse primer for cloning gDNA of OsDN-DTP19 gene
ccgctgaggccggcaccatccacatcgcgatc

SEQ ID NO: 63 - DNA, 21 bp, Artificial Sequence
  Forward primer for real-time PCR analysis of OsFBX335 gene
acgacagatgctttcctatgg

SEQ ID NO: 64 - DNA, 20 bp, Artificial Sequence
  Reverse primer for real-time PCR analysis of OsFBX335 gene
tttagcttgaccgtggaagg

SEQ ID NO: 65 - DNA, 21 bp, Artificial Sequence
  Forward primer for real-time PCR analysis of OsPK1 gene
ggaggttgcacttgttcaaag

SEQ ID NO: 66 - DNA, 23 bp, Artificial Sequence
  Reverse primer for real-time PCR analysis of OsPK1 gene
ctgacctgaatattatgggtgga

SEQ ID NO: 67 - DNA, 22 bp, Artificial Sequence
  Forward primer for real-time PCR analysis of OsDRH7  gene
agagacttgcagatgtaaaggg

SEQ ID NO: 68 - DNA, 21 bp, Artificial Sequence
  Reverse primer for real-time PCR analysis of OsDRH7 gene
aacctctgctgaaggaacatc

SEQ ID NO: 69 - DNA, 1533 bp, Oryza sativa
atggggaagctgctggacctgaagcggctcatgtccgtgccgaaggagcggaatcgtcgtcggcggcagcggcaaatccgagcccgcaatggttcaattgattcagtggctaaaagaaagggatcactctgccaacaagttgcaaattctgatggcgagagaagaacaagatattcagggcctaaccttccagaggacatctggtgccatatacactccctgatgccattcaaagatgctgcccgtgctgcatgtgtgtctcatgcttttcgtcgttcctggcaacaccgccccaatcttatattttgtattggaacactggggttggatttcatcaacaaaattgatcgtattataaaaaaccactcaggcattggcatcaaatcgctacagttagaatatgatactttttgcaatgctagaaggagcgctagcatcagctatcatctcaataattggcttcagattgctgttacaccatggattgaagaactaatccttacactgtcgctatcttcatacaatgtggattacaatttcccgtgctcacttttatctgatgggagaggaagttcattgcgacatctttatcttggcagttgtttctttcatcctacagttaatcttgaattgagaaacctgacgaggttgcatctggttactgtacatattacaggagatgagtttgggtgccttctttcgaattcttatgctttggagcgattggaacttaaatattgttatggaataatttgtctgaagataccttgcctgttgcaacgccttagccacctggaagtgtttgaatgcagaatgctgcaagccatagagaataaggctccaaatctttgcagttttgaccttggagctaggcaggtacggctcttgcttggtgaatcattgcaaatgaaaacactatccttggactatcctagtgccgtctattatgctcgtgcagagcttccatccaatgtgccaaatcttgaaattctcaccatttgctcggatcacgagatggttgatacaccaatgttgcctagcaaatttttctatctcaagtgcttgactattgatcttgcttggagactttccccagcctatgattatttctctctgatttcttttcttgacgcttctccttccttggagactttttgtctagaggaccgaatggagaatgaattgattattggagatatgtcacatatgaggcaaatgctagaacaccgtcatgacaacctccagagcgtggagattattggcttttgctatacaaagagcttgatcgagctaacatgtcatattcttgacaatacaacgtcgcttaagcaccttaaactggatactactcgtgatgtttttagctgttctacaggcaaacacgacaaatgcttccatatgggtaaggatatgctcaccgaagccaagaaagcagttttggcaattgaaacttacatcgagccaaaagttccctccactgtcatgctaaatgttgtgaaaccttgcaaccgttgccatgttgctgaatcttag

SEQ ID NO: 70 - protein, 510 aa, Oryza sativa
MGKLLDLKRLMSVPKERNRRRRQRQIRARNGSIDSVAKRKGSLCQQVANSDGERRTRYSGPNLPEDIWCHIHSLMPFKDAARAACVSHAFRRSWQHRPNLIFCIGTLGLDFINKIDRIIKNHSGIGIKSLQLEYDTFCNARRSASISYHLNNWLQIAVTPWIEELILTLSLSSYNVDYNFPCSLLSDGRGSSLRHLYLGSCFFHPTVNLELRNLTRLHLVTVHITGDEFGCLLSNSYALERLELKYCYGIICLKIPCLLQRLSHLEVFECRMLQAIENKAPNLCSFDLGARQVRLLLGESLQMKTLSLDYPSAVYYARAELPSNVPNLEILTICSDHEMVDTPMLPSKFFYLKCLTIDLAWRLSPAYDYFSLISFLDASPSLETFCLEDRMENELIIGDMSHMRQMLEHRHDNLQSVEIIGFCYTKSLIELTCHILDNTTSLKHLKLDTTRDVFSCSTGKHDKCFHMGKDMLTEAKKAVLAIETYIEPKVPSTVMLNVVKPCNRCHVAES

SEQ ID NO: 71 - DNA, 1770 bp, Zea mays
atgccgctgcgcgactgcaacgtactgatcaggaccctggccaggcgtggcagtttctcacgcgtcatggcggtgtattacgatctccgtgcgcgaggcctcgccgcagacagctacacctacccatttgtgctcatggcaatcagcacgatgaagctgtccgtcgaggggcgcaaggtgcacgcggccgcggtgaagaccggcttccgctgggatgcgtacaccacatgttcgctaatggagatgtacacgatgctgggccggagggtgttcgacaagatgccgcagcgattcctggtgctgtggaatatgatgatgagctacaaggaaaagaggtcttgccagcacaatgatgattctaaaggggaacaagaaaaaccattcctttcagagggtgtctggtctcatatacattcctttatgcacattcgagacgcggggcgagctgcctgtttgtcccggtcttttctacagtcatggagatgtcatccctacctcatttttactgatgaaacattgggttgcaagaaaaacacatctcaagaaaacgaaattgcaagcaatttttacagtagagttgataacattctgaataatcattcaggcattgggatgaaggcactcaaaatccatgttcctataggttacactgcaaaggactcacgttatcttaaccgttggcttcagattgctatcacacatgggattgaagaactcgagctaatttttcctttcatggcaaagtacaaattcccatattcccttctacctatgagtggagactcaatcaaatgtcttgaacttagttgctgttctttttgtcccacttctaagtttggctggttaaaaaatctgacaaaattgtgtcttgatggtgtttttattgagggtgacgagttagagcgccttgtcttctctgcccttgctttggagaggttggagattagatattgtgatcgcatagtctgtctgaaggttccttccatgctacaacggctcacatacctagaggtttatgattgtgacaagcttcgtgttttggacatcgatgctccaaatataacttgcttcaatttcggaagacatcatacgaagacgaaactgtcaattggtgggggagtgtccaaaataaaagaattgagtgcatgcttcgacgatgcagtttattatgtccgtgttgaactcccgtctatgatggcaaacctcgaaactcttaccataaattcaatggttgagatgaatataccaatgcttcccagcaagtatctgcacctgaagtacttaaagatttctcttgcggcgtacacctttccttcaacatttgattatttttctctggcttccttttttgatgcatgtccctctttggaggcttttttcttggatgcatatcagcggaagatgaaacatgtttcgatcattacaaatcctttggttctgagggggactcctgaacaacgccatcacaagctcaagactgttgagatgaatggattcacttccgctaagagcctggttgagctagcatgccacattgctcagtgtgcaacatcgctgcaatccctcaaattggaggtccatcagagtgattttaagtgttatattccggctaataaacataataagtgctccccgctacctgtcgaggttctgatggaatctcgacgagcacacttggccatcatgacgtacattgagcctataattccctccactgtgaagttacaggttgtaggaccttgcagcagatgccatgcccttgaacagtag

SEQ ID NO: 72 - protein, 589 aa, Zea mays
MPLRDCNVLIRTLARRGSFSRVMAVYYDLRARGLAADSYTYPFVLMAISTMKLSVEGRKVHAAAVKTGFRWDAYTTCSLMEMYTMLGRRVFDKMPQRFLVLWNMMMSYKEKRSCQHNDDSKGEQEKPFLSEGVWSHIHSFMHIRDAGRAACLSRSFLQSWRCHPYLIFTDETLGCKKNTSQENEIASNFYSRVDNILNNHSGIGMKALKIHVPIGYTAKDSRYLNRWLQIAITHGIEELELIFPFMAKYKFPYSLLPMSGDSIKCLELSCCSFCPTSKFGWLKNLTKLCLDGVFIEGDELERLVFSALALERLEIRYCDRIVCLKVPSMLQRLTYLEVYDCDKLRVLDIDAPNITCFNFGRHHTKTKLSIGGGVSKIKELSACFDDAVYYVRVELPSMMANLETLTINSMVEMNIPMLPSKYLHLKYLKISLAAYTFPSTFDYFSLASFFDACPSLEAFFLDAYQRKMKHVSIITNPLVLRGTPEQRHHKLKTVEMNGFTSAKSLVELACHIAQCATSLQSLKLEVHQSDFKCYIPANKHNKCSPLPVEVLMESRRAHLAIMTYIEPIIPSTVKLQVVGPCSRCHALEQ

SEQ ID NO: 73 - DNA, 1386 bp, Sorghum bicolor
atgccaattcaagatgctgcccgagttgcctgtgtgtctcacacctttttgcgttcctggagatgccgccctaacctcgatttcagtagggaaacattgggcttaataaatgcgtgcagaaatgaggaaacacaaaggaacatcaccagcatagttgatcacattctgcaaaaccactcaggcattggagtgaaggcagtcaagtttcaagatgattcatattacaatattcaagacttatgtcatcttgatctcgaagatgattcattttacgataatcaagacttctgtcatcttgatcttgaccgttggcttcggaatactattaaaccagggattgaagaactgaatatttctctgcatggaaagaatacaatgtacaactttccatgctcacttttatctgatgaaattggagagtcactccgtaatcttcacctcgtcggctgttactttcatcccacaatcggacttggttccttgagaaacttgagaagaatacagctgtgtaacgtatctattaccgagagtcagctcgagtgtcttctttccaattctttctctttggagcagttggtactcaggaattgcggtgggattatttgcctgaaaataccttgcctgcagcggcttagctaccttgaggtgaactcttgcagccgactggaagttatagagagcaaagctccaaatctatctagtgttcgcattgcagactacctccatgtacaactctcacttttggaaacgtcgcgtattaaaaaatattttagatcatgtcctggtgctgccttttatgctcgtactgagcttccatccagcatgccaaatcttgagtctcttagcttatcttcagcaactgagacgattaatacgccgctaatgcctagcaaattcctccacctcaagttcttgactattgctcttcgtggacagacctatgattgtttttctctgttatccttttttgatgcttctcctttcctggagactttcatattgaatgtattatcatcggtacatgtgaagcctgcctcagtttttgaagatccctcggatctgaggaagatgccagagaaccataatgacaagctcaagtgtgttcgaatcatcaatttctcatctgcaaagagcttgattgagttgacctgtcatattctcgagtctgcaatgtcactcgagtgcctcacattggacaccactcatggtgcgcctagatgctctgttaccaaacatggcagatgctggcgaatgacgagggatgaaattatggaagcgcatagagcactattggctgtccaaacatacgttaagtcaaaagttccttcaacagtcgagttaaatgttcttgagccctgcagccaatgccatgctgttgcactttag

SEQ ID NO: 74 - protein, 461 aa, Sorghum bicolor
MPIQDAARVACVSHTFLRSWRCRPNLDFSRETLGLINACRNEETQRNITSIVDHILQNHSGIGVKAVKFQDDSYYNIQDLCHLDLEDDSFYDNQDFCHLDLDRWLRNTIKPGIEELNISLHGKNTMYNFPCSLLSDEIGESLRNLHLVGCYFHPTIGLGSLRNLRRIQLCNVSITESQLECLLSNSFSLEQLVLRNCGGIICLKIPCLQRLSYLEVNSCSRLEVIESKAPNLSSVRIADYLHVQLSLLETSRIKKYFRSCPGAAFYARTELPSSMPNLESLSLSSATETINTPLMPSKFLHLKFLTIALRGQTYDCFSLLSFFDASPFLETFILNVLSSVHVKPASVFEDPSDLRKMPENHNDKLKCVRIINFSSAKSLIELTCHILESAMSLECLTLDTTHGAPRCSVTKHGRCWRMTRDEIMEAHRALLAVQTYVKSKVPSTVELNVLEPCSQCHAVAL

SEQ ID NO: 75 - DNA, 1182 bp, Oryza sativa
atgagtctaccgcttacaagaaaagatctcatgatagtcaatatgggccctcagcacccatcaatgcatggtgttcttcgactgatcgttactctcgatggtgaagatgttattgattgtgaacccatattaggctatttacacagaggaatggaaaaaatcgcggaaaaccgaactattatacaatacttaccttatgtaacacgttgggattatttagctactatgtttacagaagcaataacggtaaatgcaccagaattcttggagaatattcaaataccccaaagagccagctatattagggtaattatgttagaattgagccgtatagcttctcacttgttatggcttggaccttttatggcagatctcggtgcgcagactccttttttttatatttttagagagagagaattaatatatgatctatttgaagctgctacaggtatgcgaatgatgcataattactttcgcatcggaggagttgctgccgatctgccttatggatggatcgataaatgtttagatttctgtgattattttttacgaggagttattgaatatcaacaacttattacacagaatcccatttttttggaacgagttgagggagttggttttattagcggagaagaagctgtaaattggggcttatcgggccccatgttacgagcttctggaatacaatgggatcttcgtaaagttgatctttacgagtcttacaatcaattcgattggaaagtccaatggcaaaaagaaggggattcattagcacgctatttagtacgaatcggtgaaatgagggaatcaatcaaaattattcaacaggctgtagaaaaaattcctgggggcccttatgagaatttagaagtccgatgctttaagaaagcaaagaattccgaatggaatgattttgaatatcgatttcttggtaaaaaaccttcacccaattttgaattgtcaaaacaagagctttatgcaagagtggaagccccaaaaggtgaattaggaatttatctggtaggagatgatagtcttttcccctggagatggaaaattcgtccacccggttttattaatttgcaaattcttcctcagctagtcaaaaaaatgaaattggctgatatcatgacgatattaggtagtatagatatcattatgggagaagttgatcgttga

SEQ ID NO: 76 - protein, 393 aa, Oryza sativa
MSLPLTRKDLMIVNMGPQHPSMHGVLRLIVTLDGEDVIDCEPILGYLHRGMEKIAENRTIIQYLPYVTRWDYLATMFTEAITVNAPEFLENIQIPQRASYIRVIMLELSRIASHLLWLGPFMADLGAQTPFFYIFRERELIYDLFEAATGMRMMHNYFRIGGVAADLPYGWIDKCLDFCDYFLRGVIEYQQLITQNPIFLERVEGVGFISGEEAVNWGLSGPMLRASGIQWDLRKVDLYESYNQFDWKVQWQKEGDSLARYLVRIGEMRESIKIIQQAVEKIPGGPYENLEVRCFKKAKNSEWNDFEYRFLGKKPSPNFELSKQELYARVEAPKGELGIYLVGDDSLFPWRWKIRPPGFINLQILPQLVKKMKLADIMTILGSIDIIMGEVDR

SEQ ID NO: 77 - DNA, 1182 bp, Zea mays
atgagtctatcgcttaaaagaaaagatctcatgatagtcaatatgggccctcaacacccatcaatgcatggtgttcttcgactgattgttactctcgatggtgaagatgttattgattgcgaacccatattagggtatttacacagaggaatggaaaaaatcgcggaaaacagaagtattatacaatacttgccttatgtaacacgatgggattatttagctactatgtttacagaagcaataacggtaaatgcaccagaattcttagagaatattcaaatacccaaaagagccagctatattagagtaattatgttagaattgagccgtatagcttctcatttgttatggcttggaccttttatggcggatctcggggcacagactccctttttctacatttttagagagagagaattgatatatgatctatttgaagctgctacaggtatgcgaatgatgcataattactttcgcatcggaggagtcgctgccgatctcccttatggatggatggataaatgtttagatttctgtgattattttttacaaggagttgttgaatatcaagaacttattacacagaatcccatttttttagaacgagttgaaggagtcggttttattagcggagaagaagctgtaaattggggcttatcgggaccaatgttacgagcttctggaatacaatgggatcttcgtaaaattgatccttatgagtcttacaatcaattcgattggaaagtccaatggcaaaaagaaggagattcgttagctcgctatttagtacgagtcggtgaaatgagggaatccataaaaattattcaacaggctgtagagaaaattcctggaggaccttatgagaatttagaagcccgacgctttaagaaagcaaagaatcccgaatggaatgattttgaatatcgatttcttggtaaaaaaccttcgcccaattttgaattatcaaagcaagagctttatgtaagagtagaagctccaaaaggcgaattaggaatttatctggtaggagatgatagtcttttcccctggagatggaaaattcgtccaccgggttttattaatttgcaaattcttcctcagctagttaaaaaaatgaaattggctgatatcatgacaatattaggtagtatagatatcattatgggggaagttgatcgttga

SEQ ID NO: 78 - protein, 393 aa, Zea mays
MSLSLKRKDLMIVNMGPQHPSMHGVLRLIVTLDGEDVIDCEPILGYLHRGMEKIAENRSIIQYLPYVTRWDYLATMFTEAITVNAPEFLENIQIPKRASYIRVIMLELSRIASHLLWLGPFMADLGAQTPFFYIFRERELIYDLFEAATGMRMMHNYFRIGGVAADLPYGWMDKCLDFCDYFLQGVVEYQELITQNPIFLERVEGVGFISGEEAVNWGLSGPMLRASGIQWDLRKIDPYESYNQFDWKVQWQKEGDSLARYLVRVGEMRESIKIIQQAVEKIPGGPYENLEARRFKKAKNPEWNDFEYRFLGKKPSPNFELSKQELYVRVEAPKGELGIYLVGDDSLFPWRWKIRPPGFINLQILPQLVKKMKLADIMTILGSIDIIMGEVDR

SEQ ID NO: 79 - DNA, 1182 bp, Arabidopsis thaliana
atgaagagaccagttacaggaaaagatcttatgatagtcaatatgggacctcaccacccatccatgcatggtgttcttcgcttaattgttactctagatggtgaggatgttgttgactgtgaacccatattaggttatttacacagaggaatggaaaaaattgcagaaaaccgagcaattatacaatatttaccgtatgtaacgcgatgggattatttagctactatgtttacagaagcaataacagtaaatggaccagaacaattgggaaatattcaagttcctaaaagggccagctatatcagagtaattatgctggaattgagtcgtatagcttctcatctgttatggctcggcccttttatggcagatattggtgcacagactccctttttctatattttcagagaacgagaatttgtatatgatctattcgaagctgccaccggtatgagaatgatgcataatttttttcgtattggaggaatagcggctgatttaccttatggttggatagataaatgcttggatttttgtgattattttttaacagaggttgttgaatatcaaaaactcattacacgaaatcctatttttttagaacgggttgaaggagttgggattattggtggggaagaagcaataaattggggtttatccggaccaatgctacgcgcatccggaataccatgggatcttcgtaaaattgatcgttatgagtcttacgatgaatttgaatgggaaattcaatggcaaaaacaaggagattcattagctcgttatttagtacgacttagcgaaatgacagaatccataaaaattattcaacaggctctggaaggacttccagggggtccctatgagaatttagaaagcaggggctttgatagaaaaaggaatccagaatggaatgattttgaatatcgattcattagtaaaaaaccttctcctacttttgaattatcaaaacaagaactttatgtaagagttgaagctccaaaaggggagttgggaatttttctcataggagatcaaagcggttttccttggagatggaaaatacgaccaccgggttttattaatttgcaaattcttcctgaactagttaaaagaatgaaattggctgatattatgacgatactcggtagcatagatataattatgggagaagttgatcgttaa

SEQ ID NO: 80 - protein, 393 aa, Arabidopsis thaliana
MKRPVTGKDLMIVNMGPHHPSMHGVLRLIVTLDGEDVVDCEPILGYLHRGMEKIAENRAIIQYLPYVTRWDYLATMFTEAITVNGPEQLGNIQVPKRASYIRVIMLELSRIASHLLWLGPFMADIGAQTPFFYIFREREFVYDLFEAATGMRMMHNFFRIGGIAADLPYGWIDKCLDFCDYFLTEVVEYQKLITRNPIFLERVEGVGIIGGEEAINWGLSGPMLRASGIPWDLRKIDRYESYDEFEWEIQWQKQGDSLARYLVRLSEMTESIKIIQQALEGLPGGPYENLESRGFDRKRNPEWNDFEYRFISKKPSPTFELSKQELYVRVEAPKGELGIFLIGDQSGFPWRWKIRPPGFINLQILPELVKRMKLADIMTILGSIDIIMGEVDR

SEQ ID NO: 81 - DNA, 1308 bp, Oryza sativa
atggcgcggttggaagatatggtggaggcaaggttgagtgttctaagaggagtccaatcgagaacgaatcagacgttcaccatcttccgcgtccccgcctttcttcgcgagagcagccggacctcctacgagccgcgcttggtgtccatcggcccctactaccacggcgccgccgcgctccgcgccatggaggaccacaagtggcgctacctccacgacctgctgtctccgcaaggaccgccggaacccaaccatcgcgttccacttccaccccttgtctccggactctccgcatccgccttggttgagaagatgcggtctctcgaggccgaggcgcgcgcctgctacagcgagcaacccgtcgacctcagctccgacgacttcgtccagatgctcctcctggacggatgcttcatcctcgagttcttccgtaagtggcgcagaaatcagcccgatgtgctatgcgacgttggttggggcctcacgttcgtcatctccgatttgctccttatggagaatcagctcccgttcttcgtcctcaagaagttatatgttaccgcctttggcgaacaggatggccaggccggaaataacctcctccagcttctccttcagtacatcgccggccgtcaagttccaatcagatggccgaacgggcaggtcaatcacatcctacacctgtactacgagagcttcgtgccccagagccagcggacgccgcagcaagaacagagtaccacggcgccgcgagtgctaccgtgcgccgtcgagatgagcgaagccggggtgactttcgccgtgcggcggaactcagacaacggctacgacgtggtgttcgactcccttaggggcgtcatggagatacccaccatattgatcgacgacgcgaagacgccgctgctcgcgaacctgatcgcgttcgagcagagcctgggcaacgacgaggccatcctcctcagcagctacgtggcgctgatgggccagctcatcgtcacggcgcgcgacgtcgcgctgctccgccggcgcggcgtgctggagaacatgctggccaacgacgacgatgccgcccggttcttcaaccacctcggcgactgtggcgccgtcaaccacgacagccacgccttcgtcgggctctacaaggacgtggaccgctactgcgggacatggtggcggaggaagacggcggcgctccggcgcgactacttcgccagcccgtggtcggccatctccttcgtcgccgccgcagtcgccgtcgtgctcgcagttatgcagacctacttcaccatgtttccattgaaaaaagggtga

SEQ ID NO: 82 - protein, 435 aa, Oryza sativa
MARLEDMVEARLSVLRGVQSRTNQTFTIFRVPAFLRESSRTSYEPRLVSIGPYYHGAAALRAMEDHKWRYLHDLLSPQGPPEPNHRVPLPPLVSGLSASALVEKMRSLEAEARACYSEQPVDLSSDDFVQMLLLDGCFILEFFRKWRRNQPDVLCDVGWGLTFVISDLLLMENQLPFFVLKKLYVTAFGEQDGQAGNNLLQLLLQYIAGRQVPIRWPNGQVNHILHLYYESFVPQSQRTPQQEQSTTAPRVLPCAVEMSEAGVTFAVRRNSDNGYDVVFDSLRGVMEIPTILIDDAKTPLLANLIAFEQSLGNDEAILLSSYVALMGQLIVTARDVALLRRRGVLENMLANDDDAARFFNHLGDCGAVNHDSHAFVGLYKDVDRYCGTWWRRKTAALRRDYFASPWSAISFVAAAVAVVLAVMQTYFTMFPLKKG

SEQ ID NO: 83 - DNA, 1287 bp, Zea mays
atggaacgcgagagcgatgccggcttcttccaccacgacgacgcgcaggtggaggccatgcagcggcgcgtggacgcggcggcgcccttggccgacgacccctacaccatcttccgcctcccctcggccgtgcgcgagcgccggcgcgacctgtacgagcccaaggtcgtgtccgtgggcccgtactaccacggccgcgccggcctgggcgtcgcgcagcagcacaagtggcgcctcctccgcgacttcctctcgcggggggagaaggcggccgccgccggtggcctcggcgcgtacgtgcgcgccgcgcgcgaggtcgaggcggacgcgcgccggtgctacgccgaggggttcggcctgggcgccgacgagttcgcggagctgctggtgctcgacggctgcttcctcctcgagttcttcctcaagaagggcgaggggcagctcgccgcgcccgggggcgccaagtgggcgtggcaccacatgtaccacgacgtcctcctgctggagaaccagatacccttcttcgtcgtcgagaagctacacggcgtcgccttcgccggcgccgaccgcgaccgcgacgcgctcctggacatcttctgcaaggccttcgccggcgacctcccgtcgagccgcgtcatccggccgcgcagcgacaagaccatccaccacctgctgcacctgcactacgagtgcaacatccgcagcccagccgccgccgacagcgacaaggcccgcagcagcagcttcggcgacgccaacggcgcgtcgctggccatctggaagcagccggcgatcccgtcgccgcgctccggcgaaggcgccggcgccggcgccggcagcaaaggccgtctgacgtcgatgatcccgccggcgggcaagatggaggaggccggcgtggcgttcaagcgcaaggcggccccgcgggacctgttcgacgtcagcttccggtacggcgtgttgcacgtgccggcgttcgtgctcgacgagggcgcaaaggtgctgctcgcgaatctggtggcgttcgagcagggaggcggccgcgcggcgcggcagctggacgggggcaacctggtgacgggcttcgtggcgcttgtcgggtcgctcgtgaactcgcggagggatctggaggtgctccgccggtgcgggatcatgcactgcatgctacctgatgacgacgccgtagcctacttcaaccacgttgtacagtataccaccatggactacgaccggaaccttctcgccagcctattccgagacgttagggagcactgccattggaacagatga

SEQ ID NO: 84 - protein, 428 aa, Zea mays
MERESDAGFFHHDDAQVEAMQRRVDAAAPLADDPYTIFRLPSAVRERRRDLYEPKVVSVGPYYHGRAGLGVAQQHKWRLLRDFLSRGEKAAAAGGLGAYVRAAREVEADARRCYAEGFGLGADEFAELLVLDGCFLLEFFLKKGEGQLAAPGGAKWAWHHMYHDVLLLENQIPFFVVEKLHGVAFAGADRDRDALLDIFCKAFAGDLPSSRVIRPRSDKTIHHLLHLHYECNIRSPAAADSDKARSSSFGDANGASLAIWKQPAIPSPRSGEGAGAGAGSKGRLTSMIPPAGKMEEAGVAFKRKAAPRDLFDVSFRYGVLHVPAFVLDEGAKVLLANLVAFEQGGGRAARQLDGGNLVTGFVALVGSLVNSRRDLEVLRRCGIMHCMLPDDDAVAYFNHVVQYTTMDYDRNLLASLFRDVREHCHWNR

SEQ ID NO: 85 - DNA, 1332 bp, Sorghum bicolor
atggagcgcgagagaaacggcggcgcggcggccgcgggcgaggaggacaacggcttcttctaccacgacgacgcgcaggtggaggccatgcagcggcgcgtggacgcggcggcgcccttggccgacgacgacccctacaccatcttccgcctcccctcggctgtgcgcgagcggcaccgcgacctgtacgagcccaaggtcgtgtccgtgggcccgtactaccacggccgcgccggcctgggcgccgcgcagcagcacaagtggcgcctcctccgcgacttcctctcgcggtcgcgggggaagaataagcaggccgccgccggtggcggtggcctcggcgcgtacgtgcgcgccgcgcgcgaggtcgaggcggacgcgcgccggtgctacgccgaggggttcggcctgggcgccgacgagttcgcggagctgctggtgctcgacggctgcttccttctcgagttcttcctcaagaagggcgaggggcagctcgccgcgcccgggggcgccaagtgggcgtggcaccacatgtaccacgacgtcctgctgctggagaaccagatacccttcttcgtcatcgagaagctgcacggcgtggccttcgcggccggcggcgacgacggcggcgccgatgagcgcgacgcgctcctggacgttttctgcaaggccttcgccggcgacctgccgtcgagccgcgtcatccggccacgcagcgacaagaccatccaccacctgctgcacctgcactacgagtgcaacgtcctcaacccagccgccgatagcgacaaggcccgccgcaacagcatcggcggcgacgccaacagcgcgtcgctggccatctggaagcagccggcaatcccgtcgccgcgcaccggccagggcaggaaaggccgtctgacgtcgatgatcccgccggcgggcaagatggaggaggccggcgtgacgttcaagcgcaaggcgaccccgcgggacgtgttcgacgtcagcttccggtacggcgtgctgcacgtgccggcgttcgtgatggacgagggcgccaaggtgctgctcgcgaacctggtggcgttcgagcagggaggtggccgcgcggcgcggcagctggacgcgggcaacctggtgacgggcttcgtggcgcttgtcggatcgctcgtgaactcgcggagggacttggaggtgctccgccggtgcgggatcatgcattgcatgctggccgacgacgacgccgtggcctacttcaaccacgtggtacagtatacgaccatggactacgaccggcacctgctggcctgcctattcagggacgttcgagagcactgccattggaacagatga

SEQ ID NO: 86 - protein, 443 aa, Sorghum bicolor
MERERNGGAAAAGEEDNGFFYHDDAQVEAMQRRVDAAAPLADDDPYTIFRLPSAVRERHRDLYEPKVVSVGPYYHGRAGLGAAQQHKWRLLRDFLSRSRGKNKQAAAGGGGLGAYVRAAREVEADARRCYAEGFGLGADEFAELLVLDGCFLLEFFLKKGEGQLAAPGGAKWAWHHMYHDVLLLENQIPFFVIEKLHGVAFAAGGDDGGADERDALLDVFCKAFAGDLPSSRVIRPRSDKTIHHLLHLHYECNVLNPAADSDKARRNSIGGDANSASLAIWKQPAIPSPRTGQGRKGRLTSMIPPAGKMEEAGVTFKRKATPRDVFDVSFRYGVLHVPAFVMDEGAKVLLANLVAFEQGGGRAARQLDAGNLVTGFVALVGSLVNSRRDLEVLRRCGIMHCMLADDDAVAYFNHVVQYTTMDYDRHLLACLFRDVREHCHWNR

SEQ ID NO: 87 - DNA, 1323 bp, Arabidopsis thaliana
atggatccacagattgttgtagacataaagagtttaatctcagaagataatggtttgaagcttcttagagaatcagctggttcagagttatgttgcatcgtgagaatcccacagagcctcgctcgaatcaacctcaaagcttatgaaccgaagattgtctccattggtccttaccatcacggcaaagagcatctcaaaatgactcagcagcataaacgccgattcttgaagtttttcgtggctaaaatggaagaaaaaggatttgttcctcaagaattggtcaaagctgtatcgagtttggaaggagttatacgaggttcttactccgaggatctaggtttagattctgaaaacttggttcagatgatggttcttgatggttgctttatcctcacgttgttctttgttgtttccgggaaagttgaatacactaatcttgatgatccgatttttcgaatgccgtggattttgccgtcgattcgagcagatcttctccttttggaaaaccaggttccttatgttcttcttcaaactctatttgaaacatcgaagttagttacttgtagtggtttaaacgaaatcgcctttgaattctttaactactcattacaaaaaccagagaccttttgggaaaaacattatggtcttgaagcaaaacatcttcttgatttgatacgtaagacttttgttcctgttccgtctcaaagaagaatcaaagatcatagcagtaaatcgtcattcaatgatcatgaatatctcggatttgttctctctgccaagaagcttcatcttcggggaatcaaattcaaaccgaggaaaaacacagactcaatcttagacattagttatagtaatggtgtgcttcacattcctccggtagtcatggatgattttaccgcctcgatctttttaaactgtgtcgcatttgagcaattatatgctgattcatcgaaccacataacgagctacgtagctttcatggcttgtcttataaacgaagagagtgatgcatcgttccttagcgaaaggaggatcctagagaactattttggaacagaggatgaagtgtcccggttctataaacgcattggtaaagacattgccctcgacttagagaagagttacctagcaaaggtgtttgaaggagttaacgagtatacttctcaaggattccatgttcattgtgcagagtttattcatacgcatttcgatagtccatggacatttgcatcatcatttgcagcgttgttgcttcttctgtttgcggctttgcaagtcttctttgcagcttatagttatttccgtcctccaaaagaaaagtga

SEQ ID NO: 88 - protein, 440 aa, Arabidopsis thaliana
MDPQIVVDIKSLISEDNGLKLLRESAGSELCCIVRIPQSLARINLKAYEPKIVSIGPYHHGKEHLKMTQQHKRRFLKFFVAKMEEKGFVPQELVKAVSSLEGVIRGSYSEDLGLDSENLVQMMVLDGCFILTLFFVVSGKVEYTNLDDPIFRMPWILPSIRADLLLLENQVPYVLLQTLFETSKLVTCSGLNEIAFEFFNYSLQKPETFWEKHYGLEAKHLLDLIRKTFVPVPSQRRIKDHSSKSSFNDHEYLGFVLSAKKLHLRGIKFKPRKNTDSILDISYSNGVLHIPPVVMDDFTASIFLNCVAFEQLYADSSNHITSYVAFMACLINEESDASFLSERRILENYFGTEDEVSRFYKRIGKDIALDLEKSYLAKVFEGVNEYTSQGFHVHCAEFIHTHFDSPWTFASSFAALLLLLFAALQVFFAAYSYFRPPKEK

SEQ ID NO: 89 - DNA, 1539 bp, Glycine max
atggaacctgctactaataacaatccttctccttctgctcatgcttcattacatgaaaccaaatatattgagagggtgaaacatgccctgatcgactttggagtcccagaagagctaaggttgtctgatcgtagcatctacaaggttccttgctatctccgaaaggtgaaggaagatgcctacactcctcagtgcatctcaataggtccaattcacttcaaaaaagaagaactcaagccaatgcaagagcataagctaagatactaccaattcttcgggagacgtgttggtgtctcagacgaacaaatggaggcatacaaacactaccttgagaccgaagaaaagcagattcgacaatgctatgcagagaaattccttgacataaccaaagatacattcgtggacatgatgctgctggacgccgtattcatcatggaacttatgttgagaaactgcgagttcaaatcacacaaagccaagcatgagcaaaaccacaagcgaaccgaatcctttagaattaaaaacaacaatgatctgatcatgacacactcatggcttagcaggaacatcgctggggacttgatccttatagagaaccaaatacccttttttgtgctgcagaaactctatgatgatgttgtccctcgtgaaagcaagaaggatgaacacactgcggggtttgttaagcttgctactgagtactttgctttctatgacacccaaatgtcttcttctggtgaaaccaagaaacactgttcctgttacatactccactgtctgaaggaaccttgtaagtcaaaaggaaaagataggagtgagatttcgaaacgtcctctaggaagcaattctgaagagaatccagaaggaccaaaacatttcactgatctgataaggtggcaattttaccttcccaccgagtgtgaagccggtcatgctcatcaggttctaagaactgcaacaaagttacaaggctcagggataagctttgagaaaggtgatgtgaacaagcggttactggaaatagccttcaagaagaccccaattctgagttcatttctttgctttggttgctttccattgtcgaagctcttcaaagcgcgtttgaggattccccaattgaaggtagaccacacaactgaacgtgtgttcaagaacctcgttgcttttgagcagtttcactatccagacaagccttacttttgcaactatgtttctttcattgactctctgatacacactcagcttgatgtggagttgctggttgagaaggaagtgattgtgcatgaacttgggagtgataaggaagtggcaactcttgttaatggtttatgcaaacatgttgtcacaaactcaacttgttaccatcacattataaataagctcaacgaccattacatgaacgattggaaccacactattgcggctttaaggttggtgtacttcagagatctttggagggcaagtggcactgtggtgggtattgctgtgctggtttttgctgtcttccaattcctccgtgtttgtcgttatctcttctag

SEQ ID NO: 90 - protein, 512 aa, Glycine max
MEPATNNNPSPSAHASLHETKYIERVKHALIDFGVPEELRLSDRSIYKVPCYLRKVKEDAYTPQCISIGPIHFKKEELKPMQEHKLRYYQFFGRRVGVSDEQMEAYKHYLETEEKQIRQCYAEKFLDITKDTFVDMMLLDAVFIMELMLRNCEFKSHKAKHEQNHKRTESFRIKNNNDLIMTHSWLSRNIAGDLILIENQIPFFVLQKLYDDVVPRESKKDEHTAGFVKLATEYFAFYDTQMSSSGETKKHCSCYILHCLKEPCKSKGKDRSEISKRPLGSNSEENPEGPKHFTDLIRWQFYLPTECEAGHAHQVLRTATKLQGSGISFEKGDVNKRLLEIAFKKTPILSSFLCFGCFPLSKLFKARLRIPQLKVDHTTERVFKNLVAFEQFHYPDKPYFCNYVSFIDSLIHTQLDVELLVEKEVIVHELGSDKEVATLVNGLCKHVVTNSTCYHHIINKLNDHYMNDWNHTIAALRLVYFRDLWRASGTVVGIAVLVFAVFQFLRVCRYLF

SEQ ID NO: 91 - DNA, 1713 bp, Oryza sativa
atggcggcggcggcggctgagatcgtggggtccgcggcggcgcgcatggcggcgccggcagtgaggccggctccgcccgcggcggcggcggcggcggcgcccccgcagccgaggagggccgtggcggcgcgctccctcaggacctccacctccgacagggtggcggcggatctcgcgctcgggagcaacggctccctctccgctcagaacattgctgagaataccgctgacgctacttcgcaagtggtctctgcgaattcccgtaggaagacaaagattgtttgcaccataggcccctcaaccaacacacgcgagatgatatggaaacttgctgagactggaatgaacgtcgcacgcatgaatatgtcccatggtgaccaccagtcgcaccagaaggtgattgatttggtgaaggagtacaatgcgaagaatactgatggcaatgtcattgctattatgctggataccaagggtccagaagtgagaagtggggatgttccagaaccaatcatgctcgaggaaggtcaagagttcaattttactattaaaagaggggtgagcaccaaagacaccgtcagtgtgaattacgatgacttcataaacgatgttgaagttggggacatactgttggtggatggaggaatgatgtcactcgctgtcaagtctaaaacagctgatacggttaagtgtgaagtagttgatggtggggaactgaaatcaaggcgccacctaaatgtccgtggaaagagtgcgactttgccatctattacagagaaagattgggaggacataaagtttggtgttgaaaatggtgttgatttctatgccgtttcctttgtgaaggatgcaaaagttattcatgaactgaaggactaccttaaaagtgctaatgcagatatacatgtcattccaaagattgaaagtgcagattcaataccaaacctccagtccattattgctgcttcagatggggcgatggtggcaagaggggatcttggtgctgaacttccaattgaggaagttcctttgctgcaggaggagattgtcagaacatgccgaagcatgcagaaaccagttattgttgccacgaatatgttagagagcatgatagaccatcccactccaacaagagcagaagtttctgatatagcaattgcagttcgtgaaggttctgatgccatcatgctgtctggtgaaactgcccatggaaagttcccactgaaggcagtcaaggtgatgcacacagtggcacagagaacagaatccagcctgtataacccaactacatctcctagtcttgttgcacatcctcaggctctgctcaacgaggaattttcgcaaagccaactaagcaaaatgtttggatctcatgctacaatgatggcgaacaccctttgcaccccaattattgtgtttacacgaaccggctccatggcagtccttctcagccactaccggccctcgtctacaatttttgcatttacaaatgaggaacgagtgaagcaacgtctggcactctaccagggtgtggttcccatttacatgaagttttctgatgatgcagaggaaactttctctagagcaattagtagcttgctgaacgcccaattcgtgaaagaaggggactacgtgacccttgttcagagtggagtgaagtcgatctggagagaggagtctactcaccacattcaagtgaggaaagtccagggctaa

SEQ ID NO: 92 - protein, 570 aa, Oryza sativa
MAAAAAEIVGSAAARMAAPAVRPAPPAAAAAAAPPQPRRAVAARSLRTSTSDRVAADLALGSNGSLSAQNIAENTADATSQVVSANSRRKTKIVCTIGPSTNTREMIWKLAETGMNVARMNMSHGDHQSHQKVIDLVKEYNAKNTDGNVIAIMLDTKGPEVRSGDVPEPIMLEEGQEFNFTIKRGVSTKDTVSVNYDDFINDVEVGDILLVDGGMMSLAVKSKTADTVKCEVVDGGELKSRRHLNVRGKSATLPSITEKDWEDIKFGVENGVDFYAVSFVKDAKVIHELKDYLKSANADIHVIPKIESADSIPNLQSIIAASDGAMVARGDLGAELPIEEVPLLQEEIVRTCRSMQKPVIVATNMLESMIDHPTPTRAEVSDIAIAVREGSDAIMLSGETAHGKFPLKAVKVMHTVAQRTESSLYNPTTSPSLVAHPQALLNEEFSQSQLSKMFGSHATMMANTLCTPIIVFTRTGSMAVLLSHYRPSSTIFAFTNEERVKQRLALYQGVVPIYMKFSDDAEETFSRAISSLLNAQFVKEGDYVTLVQSGVKSIWREESTHHIQVRKVQG

SEQ ID NO: 93 - DNA, 1728 bp, Zea mays
atggcggcgcaggtggtcgcagcggcgggcaccccggtggccaggccgctgggcggtggatctggggccgacgcgctccggcccgcggcgaggatgccaagggagagaaggaccggtgctgtggccgcgagaggacgccgcgagtctcaggtggtatccgtcataagccgcgccccacgccccgatgccggggtgctgccggtgtcgcccgacgacgacgcggccgtaaaggaagaagcaaacttccagcaccttaaggctatccagcaacttgcaactgcagcaaatggcgtgtggtctaaaccaaatgtaaggcgcaagacaaagattgtgtgtacaatcggtccttcaaccaacacaagggacatgatatggaaactcgctgagactggcatgaatgtggctcggcttaatatgtcacatggagaccatgcatcacaccagaaagttattgatctggtaaaggagtacaatgcatcacatgctgacaatgtgattgctatcatgcttgacacaaagggaccagaagttcgaagtggagatttgcctcaaccgatatttctggaaagcggacaagaatttacttttacaatcaaaaggggagttgggactgatacgtgtgttagcgttaactatgacgactttgttaatgatgttgaagtgggcgacatgctccttgtagatggaggaatgatgtcattcttggtcaaatcaaaaactgaagattctgtgaaatgtgaagttattgacggtggtgaattgaaatctaggcgccatctgaatgttcgtggaaagagtgcaaccttgccatcaataactgacaaggactgggacgacattaagtttggtgtggataatcaagttgattactatgctgtttcctttgtgaaagatgctcaagttgtccacgaactcaaggattatctaaaaagttgcaatgctgacatacatgttatcgtaaaaatagaaagtgcagactccatccccaacttacattcaatcatcacagcatctgatggggctatggttgccaggggtgaccttggagccgagctgcccattgaggaggtgccgctgttgcaggaagaaattattagaatgtgcaggagcatggggaaggctgttattgttgctacaaatatgctcgaaagtatgattgttcatccaactccaacccgagcagaagtttcagacattgctatagctgtccgagagggtgctgatgcagttatgctttcaggagaaactgcacatgggaaatttcccttgaaagctgttaaggtcatgcacactgttgccctgagaaccgaggcaactattcctggtggggaaacacctgcagaccttggtcaggctttcaagaaccacatgagcgaaatgttcgcataccatgcaacaatgatgtcaaatacccttcgaacatcaatagtggttttcactaggacgggatttatggctatactgcttagtcactaccgtccatctggcactatttttgcctttacagatgaggagagggttaggcaacgattggctttgtaccaaggtgtatgtccggttcaaatggaattttctgatgatgctgagaagacatttggcaatgctttgtcttatttgctgaaacatggtatggttaaggacggtgaggaggttgcgctcgttcaaagtggtaaacatcccatctggagatcacaatcaacacacaacattcaggtgaggaagatctga

SEQ ID NO: 94 - protein, 575 aa, Zea mays
MAAQVVAAAGTPVARPLGGGSGADALRPAARMPRERRTGAVAARGRRESQVVSVISRAPRPDAGVLPVSPDDDAAVKEEANFQHLKAIQQLATAANGVWSKPNVRRKTKIVCTIGPSTNTRDMIWKLAETGMNVARLNMSHGDHASHQKVIDLVKEYNASHADNVIAIMLDTKGPEVRSGDLPQPIFLESGQEFTFTIKRGVGTDTCVSVNYDDFVNDVEVGDMLLVDGGMMSFLVKSKTEDSVKCEVIDGGELKSRRHLNVRGKSATLPSITDKDWDDIKFGVDNQVDYYAVSFVKDAQVVHELKDYLKSCNADIHVIVKIESADSIPNLHSIITASDGAMVARGDLGAELPIEEVPLLQEEIIRMCRSMGKAVIVATNMLESMIVHPTPTRAEVSDIAIAVREGADAVMLSGETAHGKFPLKAVKVMHTVALRTEATIPGGETPADLGQAFKNHMSEMFAYHATMMSNTLRTSIVVFTRTGFMAILLSHYRPSGTIFAFTDEERVRQRLALYQGVCPVQMEFSDDAEKTFGNALSYLLKHGMVKDGEEVALVQSGKHPIWRSQSTHNIQVRKI

SEQ ID NO: 95 - DNA, 1743 bp, Sorghum bicolor
atggcggcgcaggtggtggcagcggcgggcaccgcggtggccaggccgctgggcggcggatctgggctcgacgcggcgccccgtggcggcggcgtggcgaggatgccaagggagaggtggggcggcgctgtggccgcgaggggacgccgcgaccgcgagtcgcaggtggtgtccgtcataagctgcgccccgcgctcccaggccgaggtgctgccggtgtcgcccgacgacgacgcggccgtaaaggaagaagcaaacttccagcaccttaaggctatccagcagcttgcaactgcagcaaatggtgtgtggtctaaaccaaatgtaaggcgcaagacaaagatcgtgtgtacaattggtccttcaactaacacaagggagatgatatggaaacttgccgaggctggcatgaatgtggctcggcttaatatgtcacatggagaccatgcgtcacaccagaaagttattgatctggtgaaggagtataatgcatcacatgctgacaatgtgattgctatcatgcttgacacaaagggaccagaagttcgaagtggagatttgcctcaaccgatatttctggaaagtgggcaagagttcacttttacaatcaaaaggggagttgggactgatacatgtgttagcgttaactatgatgactttgttaatgatgttgaagttggtgacatgctccttgtagacggaggaatgatgtcattcttggtcaaatcaaaaactgaagattctgtgaaatgtgaagttattgatggtggtgaattgaaatctaggcgccatctgaatgttcgtggaaagagtgcaaccttgccatcaataactgacaaggattgggacgacattaagtttggtgtggataatcaagttgattactatgctgtttcttttgtgaaagatgctcaagttgtccatgaactcaaggattatctaagaagttgcaatgccgacatacatgtaattgtaaaaatcgaaagtgcggactccatcccaaacctacattcaatcatcacagcatctgatggggctatggttgccaggggtgaccttggagctgaactgcccatcgaggaggtgccgctgttgcaggaagaaattattagaatgtgtaggagcatggggaaggctgttattgtcgctacaaatatgctggaaagtatgattgttcatccaactccaacccgagcagaagtttcagacattgctatagctgtccgagagggtgctgatgcagtcatgctttctggagaaactgcacatgggaaatttcccttgaaagctgttaaggtcatgcacactgttgccctgagaactgaggcaactattcctggtggagaaacacctgcagaccttggtcaggctttcaagaaccacatgagtgaaatgtttgcgtaccatgcaacaatgatgtcaaatacccttcgaacatcaatagtggttttcactaggacaggatttatggctatactgcttagtcactaccgtccatctggcactatttttgccttcacagatgaggagagagttaggcaacgattggctttgtaccaaggtgtatgtccaattcaaatggaattttctgatgatgctgagaagacatttggcaacgctttgtcttatttgctgaaacatggtatggttaaggatggtgaggaggtcgcactccttcaaagtggtaatcagcccatctggagatcacaatcaacacacaacattcaggtgaggaagatctga

SEQ ID NO: 96 - protein, 580 aa, Sorghum bicolor
MAAQVVAAAGTAVARPLGGGSGLDAAPRGGGVARMPRERWGGAVAARGRRDRESQVVSVISCAPRSQAEVLPVSPDDDAAVKEEANFQHLKAIQQLATAANGVWSKPNVRRKTKIVCTIGPSTNTREMIWKLAEAGMNVARLNMSHGDHASHQKVIDLVKEYNASHADNVIAIMLDTKGPEVRSGDLPQPIFLESGQEFTFTIKRGVGTDTCVSVNYDDFVNDVEVGDMLLVDGGMMSFLVKSKTEDSVKCEVIDGGELKSRRHLNVRGKSATLPSITDKDWDDIKFGVDNQVDYYAVSFVKDAQVVHELKDYLRSCNADIHVIVKIESADSIPNLHSIITASDGAMVARGDLGAELPIEEVPLLQEEIIRMCRSMGKAVIVATNMLESMIVHPTPTRAEVSDIAIAVREGADAVMLSGETAHGKFPLKAVKVMHTVALRTEATIPGGETPADLGQAFKNHMSEMFAYHATMMSNTLRTSIVVFTRTGFMAILLSHYRPSGTIFAFTDEERVRQRLALYQGVCPIQMEFSDDAEKTFGNALSYLLKHGMVKDGEEVALLQSGNQPIWRSQSTHNIQVRKI

SEQ ID NO: 97 - DNA, 1740 bp, Arabidopsis thaliana
atggctcaagtggttgctaccaggtcaattcaaggctcgatgttatctcccaacggtggatctgtgtctacaagatccgagaagctattgaaaccagcgagttttgcagtgaaggttcttggcaacgaagcaaagagaagtggaagagtctctgtaagaagcagaagagtggttgatactactgtgagatccgctcgtgttgagactgaagtcattcctgtttctcctgaagatgtgcctaacagagaggagcagcttgagaggttgttggaaatgcagcagtttggtgatacatcggtagggatgtggtcgaagccgacagtgaggaggaagacaaagattgtttgcaccgttggtccgtcgaccaacacacgagaaatgatatggaaattggctgaagctgggatgaatgttgctaggatgaatatgtctcatggagatcatgcttcacataagaaggttattgatttggttaaagaatacaatgcacaaactaaagacaacactattgctatcatgcttgacaccaagggtccggaagttaggagtggagatttacctcagccaattatgttagatcctggtcaagagtttacctttacaattgagagaggagtcagcacaccaagttgtgtcagtgttaactatgatgatttcgttaatgacgtggaagcgggtgacatgcttcttgttgatggtggtatgatgtcgtttatggtgaagtcaaagaccaaagactctgtcaaatgtgaagttgttgatggtggagaacttaagtcaaggagacacctgaatgtccgaggaaagagtgcaactttaccttcaatcactgagaaggactgggaggatattaaatttggagtggagaacaaagttgacttttatgcagtttcctttgtcaaagatgctcaagttgtacacgagttgaagaaataccttcaaaatagtggtgctgatatacacgtgatagtgaaaattgagagtgcagactccatacctaacttgcactccattatcacagcatcagatggggcaatggttgcaagaggtgatcttggtgcagagcttccaattgaagaagtccccattcttcaggaggagatcattaacctgtgccgtagtatgggaaaagctgttattgttgcgactaacatgcttgagagtatgatagttcatccaactccaacccgggcagaggtctcagacattgctatcgctgttagagaaggtgctgatgcggtaatgctttcaggagaaactgctcacggaaagttcccattgaaagctgctggagtgatgcacactgttgcattgcgaacagaagcaaccattactagcggtgaaatgccacctaatcttggtcaagccttcaagaaccatatgagtgagatgtttgcataccatgcaaccatgatgtcaaacacacttggaacttcaactgttgtcttcaccagaaccggtttcatggccatattgttaagtcactatcgtccttccggcacaatctatgccttcacaaatgagaaaaaaatacaacaaagattagctttgtatcaaggtgtatgccccatatatatggagttcacagatgatgcagaagaaacttttgctaatgctttggctacattactgaaacaaggaatggtgaagaagggagaggaaatagcaatcgtacagagcggtacacagccaatctggcgatctcaatcgacacataacatccaagtccgcaaggtttaa

SEQ ID NO: 98 - protein, 579 aa, Arabidopsis thaliana
MAQVVATRSIQGSMLSPNGGSVSTRSEKLLKPASFAVKVLGNEAKRSGRVSVRSRRVVDTTVRSARVETEVIPVSPEDVPNREEQLERLLEMQQFGDTSVGMWSKPTVRRKTKIVCTVGPSTNTREMIWKLAEAGMNVARMNMSHGDHASHKKVIDLVKEYNAQTKDNTIAIMLDTKGPEVRSGDLPQPIMLDPGQEFTFTIERGVSTPSCVSVNYDDFVNDVEAGDMLLVDGGMMSFMVKSKTKDSVKCEVVDGGELKSRRHLNVRGKSATLPSITEKDWEDIKFGVENKVDFYAVSFVKDAQVVHELKKYLQNSGADIHVIVKIESADSIPNLHSIITASDGAMVARGDLGAELPIEEVPILQEEIINLCRSMGKAVIVATNMLESMIVHPTPTRAEVSDIAIAVREGADAVMLSGETAHGKFPLKAAGVMHTVALRTEATITSGEMPPNLGQAFKNHMSEMFAYHATMMSNTLGTSTVVFTRTGFMAILLSHYRPSGTIYAFTNEKKIQQRLALYQGVCPIYMEFTDDAEETFANALATLLKQGMVKKGEEIAIVQSGTQPIWRSQSTHNIQVRKV

SEQ ID NO: 99 - DNA, 1728 bp, Glycine max
atgtctcaggtagtggccactcgatccattcactcctccctcacgcgccccacctcaggatctgcacaccacagggcccaaacgttgttgaagcctccaacttttgcttccaaattgttcggagcacaaaggaacaacccctccaaagtttgctcccgaagttgcctcgtcaatgcgaggaaatctgcacccgctaaagttgttcccgtgtcacccgaggatgattcaaagattgaggaagagttgcagcacttgcgtggtatgcagcaacttggcgacacttctgttggaatgtggtcaaaacccacgtttaggaggaagacaaaggttgtttgcaccattggtccttctaccaacaccagggaaatgatttggaagctggctgaggctgggatgaatgttgcccgattgaatatgtctcacggagaccatgcttctcatcagaaaattattgatttggttaaagaatataatgctcaatccaaggacaacgtaattgcaattatgcttgataccaagggtcctgaggttaggagtggggatttgccacaaccaatcaatttaacaactgggcaggaattcacttttaccatccggaggggtgttggaactgcagattgtgttagtgtgaactatgacgatttcgtcaatgatgtggatgtgggagacatgcttcttgttgatggtggtatgatgtctttggtggttaagtctaagacagaggattctgtgaaatgtgaagttgttgatggaggagagctcaagtcaaggagacatttgaatgttagaggaaaaagtgcaacactgccttccataactgagaaggattgggatgacatcaaatttggagtggataacaaagttgacttctatgctgtttcttttgttaaggatgcacaagtagttcatgaactgaagaattatttgaaaagctgtgatgctgatatacacgtcattgtaaaaattgaaagtgcagactctataccaaacttgcattcaattattacagcgtctgatggggccatggttgcaagaggagatcttggtgcagaactccctattgaagaggttccacttttgcaggaagaaataatcaccatatgtcgtagcatgggaaaggccgttattgtggcaacaaatatgctggaaagcatgattgttcacccgacaccaaccagagccgaggtatccgatattgcaattgctgttcgagaaggttctgatgcaataatgctttctggggaaactgctcatggaaagttcccactaaaagccgtgaaagtaatgcacaccgtagcattacggacagaagccactatacctggtggtcaaatgccaccaaatattggtcaagtattcaagaaccacatgagtgagatgtttgcttaccatgcaaccatgatgtctaatacccttggaacctcaactgttgtcttcactagatcaggcttcatggctatccttttgagccactatcgaccttcaggcaccatatttgcttttacagatcaaaagaggatacaacagaggttggctttgtatcaaggagtctgtcctatttacatggaattctctgaagatgctgaagagactttcacaagggccttggatttgctgcagaagcaaggaatggtgaaatcaggagaagaagtagcactagtacaaagtggcacgcaacccatatggaggttccaatccactcacaatatccaggtccgaacagtgtaa

SEQ ID NO: 100 - protein, 575 aa, Glycine max
MSQVVATRSIHSSLTRPTSGSAHHRAQTLLKPPTFASKLFGAQRNNPSKVCSRSCLVNARKSAPAKVVPVSPEDDSKIEEELQHLRGMQQLGDTSVGMWSKPTFRRKTKVVCTIGPSTNTREMIWKLAEAGMNVARLNMSHGDHASHQKIIDLVKEYNAQSKDNVIAIMLDTKGPEVRSGDLPQPINLTTGQEFTFTIRRGVGTADCVSVNYDDFVNDVDVGDMLLVDGGMMSLVVKSKTEDSVKCEVVDGGELKSRRHLNVRGKSATLPSITEKDWDDIKFGVDNKVDFYAVSFVKDAQVVHELKNYLKSCDADIHVIVKIESADSIPNLHSIITASDGAMVARGDLGAELPIEEVPLLQEEIITICRSMGKAVIVATNMLESMIVHPTPTRAEVSDIAIAVREGSDAIMLSGETAHGKFPLKAVKVMHTVALRTEATIPGGQMPPNIGQVFKNHMSEMFAYHATMMSNTLGTSTVVFTRSGFMAILLSHYRPSGTIFAFTDQKRIQQRLALYQGVCPIYMEFSEDAEETFTRALDLLQKQGMVKSGEEVALVQSGTQPIWRFQSTHNIQVRTV

SEQ ID NO: 101 - DNA, 1788 bp, Oryza sativa
atggttgaaagtgcaagcatggtgaacgagaactcggagaatccatactggaaagcaataggatacagagtggaagagccccgacgtgatcgagcagagtcgatgccgtcgccgtcgccatcgccggtatcgcggcggccactggacaacggcgtcgtggagacgagggcgctgacggacaccaccctcctccggtcgctcgcggcgaagggcctcgccgtgaggcccggcgcgtcggacgagcaccacacggtgcggtgcgacgccgtcatcgtcggctccggctgcggcggcggcgtggccgccgcggtgctcgcgtccgccgggtacaaggtggtcgtcgtcgagaagggcgactacttcaccaaggaggattacagctcgatcgagggcccgtccatggagcgcctcttcgagaggggcggcgtcttctgcacgtccaacgtcacgacgatgatattcaccggcgcgacggtcggcggcgggtcggcggtgaactggtcggcgagcatccgcacgccggcgggcgtgatgcaggagtggtcgcgcgagcacgggctggcggtgttcgcgagccccgggtacgcgcgggccatggacgcggtgtgcgagcgcctcggtgtgaccgacgcgtgccgggaggaagggttccagaacaaggtggtgcgccgcgggtgcgacgcgctcgggctgcgcgccgacgccgtgccgcgcaactcgtcggaggggcacttctgcggcagctgcaacttcgggtgccccaccggcgacaagaagggcaccgacacgacgtggctcgtcgacgccgtcgagcgcggtgcggtcatcctgaccgggtgcaaggccgaacacttcatcgtcgagagcaacggcggtggcggcggccggagcaagaggtgcgtcggcctggtggcgacgtgcatgagcaacggcatcaccaagaagctccgcgtcgaggcgaaggtgtccatctcggcgagcggcgcgctcatgacgccgccgctgctgcgcaacagcgggctcaagaaccgccacatcggccggaacctgcacctccacccggtgtccatggcgtggggctacttcccggacaacacgccggagccgcacatcccggggaagtgctacgagggcggcatcatcaccagcatgcaccgcgtcacggagcgcaccatcatcgagacgccagcgctcggcccgggcgccttcgccgccctggtgccctgggagtccggccgcgacatgaaggagcggatgcgccggtacgcgcgcacggcgcacgcgttcgcgctggtgcgcgaccgcggcgccgggtccgtcgacggcgagggccgcgtccgctacgccccgagccgcgacgacgccgaggagctccgcgccggcctccgccgcgcgctgcgcatcctggtggccgccggcgccgccgaggtgggcacgcaccgcagcgacggggcccgcctccgatgcaagggcgcgcgcgacgcggacgtggaggcgttcctcgacgaggtgaccgtggagaaggggccgatgcactcgacgacggacaagtggtcggtgctctgctcggcgcaccagatggggagctgccggatgggcgcgagcccccgcgacggcgccgtcgacgtcgccggcgagagctgggaggcggaggggctctacgtctgcgacggcagcctgctcccgacggcggtgggcgtgaacccgatgatcaccatacagtccatcgcctactgcgtcgccaagggcatagccgactcgatggcacacggcaaggagcagcgctag

SEQ ID NO: 102 - protein, 595 aa, Oryza sativa
MVESASMVNENSENPYWKAIGYRVEEPRRDRAESMPSPSPSPVSRRPLDNGVVETRALTDTTLLRSLAAKGLAVRPGASDEHHTVRCDAVIVGSGCGGGVAAAVLASAGYKVVVVEKGDYFTKEDYSSIEGPSMERLFERGGVFCTSNVTTMIFTGATVGGGSAVNWSASIRTPAGVMQEWSREHGLAVFASPGYARAMDAVCERLGVTDACREEGFQNKVVRRGCDALGLRADAVPRNSSEGHFCGSCNFGCPTGDKKGTDTTWLVDAVERGAVILTGCKAEHFIVESNGGGGGRSKRCVGLVATCMSNGITKKLRVEAKVSISASGALMTPPLLRNSGLKNRHIGRNLHLHPVSMAWGYFPDNTPEPHIPGKCYEGGIITSMHRVTERTIIETPALGPGAFAALVPWESGRDMKERMRRYARTAHAFALVRDRGAGSVDGEGRVRYAPSRDDAEELRAGLRRALRILVAAGAAEVGTHRSDGARLRCKGARDADVEAFLDEVTVEKGPMHSTTDKWSVLCSAHQMGSCRMGASPRDGAVDVAGESWEAEGLYVCDGSLLPTAVGVNPMITIQSIAYCVAKGIADSMAHGKEQR

SEQ ID NO: 103 - DNA, 2307 bp, Zea mays
atgggcgcgggcaacaacaagcggggacaccctctcctgaggggtgggagcgcgaagagggagcggtacacgcacggcttctcggcgtcccagatggccgcgctcaccgccctctgcggcgcgctggtgccgtccctgccgccggcggaccgccggaacggtcaccaccacaacccgcagcctgaggaggacggcggcagcagcaaggtcgtggaggagttcctcctcgcgtcggccgccgacccgcccgtgcccggcgaggtggccgagctgatgtcgcgcaagtgcctccccgaggcgctggctctggtgcgcgcggtgctgtggctgctgggcacgcggctcggctcgctggcgctgtgcggggcgcggtgcctgtcgtggcggttcccgttcgtgcgccggttcgacgagctgcccctggagcaccgggaggccgcgctccggcggtggagccggctgaccctgctcccgccgctgcgcatgttcttcctcatcaccaaggtcttctgcctctacgttttctactcctggaccgacgagaactcaaagaatccgcactggcgcgcgatcgggtacagcccgcccccggcggacgacgagccggcgccggcggaatcagaacgtcccgagaagcgtcccctggacgacggcatcgttgagacgaacaaggagaacgacgcgtccctgcccgcgttgctagccgagaagggcctcagggtggccgatgacgcggcgcggaacgtgtgcagggtcgagtgcgacgtcgtcatcgtcgggtccggctgcggcgggggcgtggcggcagcggtgctcgcggcggcggggcacaaggtggtcgtcatcgagaaggggaactacttcacggcgcgggattacacggccatcgaggcgccgtcgatggagcacctctacgagggcggagggttcgtcagcacgctcagcgccagcgcgctgctcctggcaggctcgacggtcggcggcggcacggcggtcaactggtcggcctgcatcaagacccccgacgacgttcgcggggagtgggcgcgcgaccgggggctcccgctgttcgccaccggcgagtacgccgccgccatggacaaggtgttcgagcggctcggcgtgacggccggttgtgcggaggaggggctccagaacaaggtcctccgcaaggggtgcgagaggctcgggtacaaggtggagtcggtgtcgaggaactcgtcggaggggcactactgcggcagctgcggctacggctgccgcaccggcgacaagcgcggcacggacagcacgtggctcgtcgacgcggtgagccgcggcgcggtgatcctgacggggtgcaaggccgagaagctgctgctggagcctggcagtgccgcggacggcagggcgaagcgatgcgtcggcgtggtggcgaggagcaccaacccggcgatcacgaggacgctggaggtgaccgccagggtgacagtctcggcgtgcgggtctctcctgacgccggtgctgctgcgcgggagcgggctccgcaaccggcatatcgggaagaacctccacctccacccgacggccctggtgtggggctacttcccggacaccgtgccggacctcaggggcaggatgtacgagggcggcatcatcacgtccctgcacaaggtggaaggcggcggtccgggcgcgccggcccgcgccatcctcgaggcgccggcgatgggcctggccggcgcggggacgcagttcccctgggtgtccgggcgcgacatgaaggagcggatgctcaggtacgggcgcacggtgcacctcttctccatggtgagggaccgcgggtccggcaccgtgcacggcgagcggcgggtggcgtaccacctggacgccacggaccgggagaacatgcgcgacggcatgcggcgcgcgctgcgcgtcctggccgcggcgggcgcggcggagatcggcacccaccgcagcgacgggcagcggttcgcgtgcgggggcgccacggaggccgcgctggaggagttcctggacggcgtggacgtggtgcgcgggccccagtcgaaggccgaggcgtggaccctgtgctgcaccgcgcaccagatgggcagctgccggatgggcgccacggcccgggacggcgccgtggacgcgcgcggcgagagctgggaggccgggagcctgtacgtgtgcgacggcagcgtcctgcccggcgccgtgggcgtcaaccctatggtcaccatccagtccgtggcctactgcctcgccacgggcatcgccgagtcgctcaggcgcggcccggtttcacgaaaggattga

SEQ ID NO: 104 - protein, 768 aa, Zea mays
MGAGNNKRGHPLLRGGSAKRERYTHGFSASQMAALTALCGALVPSLPPADRRNGHHHNPQPEEDGGSSKVVEEFLLASAADPPVPGEVAELMSRKCLPEALALVRAVLWLLGTRLGSLALCGARCLSWRFPFVRRFDELPLEHREAALRRWSRLTLLPPLRMFFLITKVFCLYVFYSWTDENSKNPHWRAIGYSPPPADDEPAPAESERPEKRPLDDGIVETNKENDASLPALLAEKGLRVADDAARNVCRVECDVVIVGSGCGGGVAAAVLAAAGHKVVVIEKGNYFTARDYTAIEAPSMEHLYEGGGFVSTLSASALLLAGSTVGGGTAVNWSACIKTPDDVRGEWARDRGLPLFATGEYAAAMDKVFERLGVTAGCAEEGLQNKVLRKGCERLGYKVESVSRNSSEGHYCGSCGYGCRTGDKRGTDSTWLVDAVSRGAVILTGCKAEKLLLEPGSAADGRAKRCVGVVARSTNPAITRTLEVTARVTVSACGSLLTPVLLRGSGLRNRHIGKNLHLHPTALVWGYFPDTVPDLRGRMYEGGIITSLHKVEGGGPGAPARAILEAPAMGLAGAGTQFPWVSGRDMKERMLRYGRTVHLFSMVRDRGSGTVHGERRVAYHLDATDRENMRDGMRRALRVLAAAGAAEIGTHRSDGQRFACGGATEAALEEFLDGVDVVRGPQSKAEAWTLCCTAHQMGSCRMGATARDGAVDARGESWEAGSLYVCDGSVLPGAVGVNPMVTIQSVAYCLATGIAESLRRGPVSRKD

SEQ ID NO: 105 - DNA, 2322 bp, Sorghum bicolor
atgggcgcgggcgacaagcggggacaccctctcctgaggggaggagccgcgaacagggagcggtacacccacggcttctcggcgtcccagatgaccgcgctcaccgccctctgcggcgcgttggtgccgtccctgccgccggaccgccggaacggtcaccagcagcaggaggacggcggccgcggcgggggcagcggcggcaacaaggacaaggtcgtggaggagttcctcctcgcctcggccgccgacccgcccgtgcccggcgaggtggccgagcagatgtcgcgcaagtgcctccccgaggcgctggcgctggtacgcacggtgctgtggctgctgggcacccggctcggctcgctggcgctgtgcggggcccggtcgtgcctgtcgtggcggttcccgttcgtgcgccggttcgacgagctgcccctggagcagcgggaggccgcgctccggcggtggagccggcagaccctgctcctgccgctgcgcatgttcttcgtcattatcaaggccatctgcctccacgtcttctactcctggaccgatgagaactcaaagaatccgcactggcgggcgatcgggtacagcccgccgctggccgacgacgagccggcgccggcggaatcagagcgtcccgagaagcgtcccctcgacgacggcgtcgtggagacgaccaaggagaccgacgcgtccctgccggcgttgctagcggcgaagggcctcacggtggccgatgactcggcgcagaacgtgtgcagggtcgagtgcgacgtcgtcatcgtcggttccgggtgcggcgggggcgtggcggcagcggtgctcgcgggggcggggcacaaggtggtcgtcatcgagaaaggaaactacttcacggcgcgggattacacggctatcgaggcgccgtcgatggagcagctctacgagggcggaggtgccgtcagcacgctcagcggcagcgcgctagtcctggcaggctctacggttggcggcggtacggcggtgaactggtcagcttgcatcaagacccctgacgacgttcgcgaggagtgggcgagcgaccaggggctcccgttgttcgccaccgacgagtacgccgccgccatggacaaggtgttcgagcggctcggcgtgacggccgggtgcgcggaggaggggctccagaacaaggtcctccgcaaggggtgcgagaagctcgggtacaaggtggagtcggtgtcaaggaactcgtcggaggggcactactgcggcagctgcggttatggctgccgcagcggcgacaagcgcggcacggaccgaacgtggctggtcgacgcggtgagccgcggcgcggtgatccttacgggatgcaaggccgagaagctgctgcttgagcgtactggcactggcggcgcggagagcagggcgaagcgatgcgtcggcgtggtggcgaggagcaccaacccgacgatcacgaggacgctggaggtgcgcgccagggtgaccgtctcggcgtgcgggtcgctcctgacgccggtgctgctgcgcgggagcgggctcagcaaccggcacatcggcaagaacctccacctccacccgacggccctggtgtggggctacttcccggacaccatgccggacctcaagggcaagacgtacgagggcggcatcatcacgtccctgcacaaggtggaaggcgctccgggcgcgccggcccgcgccatcctcgaaacgccggcgatgggcctggccggcgcggggacgctgttcccttgggtgtccgggagcgatttgaaggagcggatgctcaggtacggccgcacggtgcacctcttctccctggtgagggaccgcggttccggcagcgtgctcggagagcggcgggtcgcgtaccacttggacgccacggacgcggagaacatgcgcgagggcctgcgccgcgcgctgcgcatcctcgccgcggcgggcgcggcggagatcggcacccaccgcagcgacgggcagcggttcgcgtgcaggggcgccacggaggcggcgctggaggagttcctggacggcgtggacgtggtgcgcgggccccagtcgaaggccgaggcgtggagcctgtgctgcacggcgcaccagatgggcagctgccggatgggcgtcacggcccgggacggcgccgtggacgcgcgcggcgagagctgggaggtcgagagcctgtacgtgtgcgacggcagcgtcctacccagcgccgtgggtgtcaaccccatggtcaccatccagtccgtggcctactgcctcgccacgggcatcgccgagtcgctcaggcgtggcccggttcccgaaaagatccagtga

SEQ ID NO: 106 - protein, 773 aa, Sorghum bicolor
MGAGDKRGHPLLRGGAANRERYTHGFSASQMTALTALCGALVPSLPPDRRNGHQQQEDGGRGGGSGGNKDKVVEEFLLASAADPPVPGEVAEQMSRKCLPEALALVRTVLWLLGTRLGSLALCGARSCLSWRFPFVRRFDELPLEQREAALRRWSRQTLLLPLRMFFVIIKAICLHVFYSWTDENSKNPHWRAIGYSPPLADDEPAPAESERPEKRPLDDGVVETTKETDASLPALLAAKGLTVADDSAQNVCRVECDVVIVGSGCGGGVAAAVLAGAGHKVVVIEKGNYFTARDYTAIEAPSMEQLYEGGGAVSTLSGSALVLAGSTVGGGTAVNWSACIKTPDDVREEWASDQGLPLFATDEYAAAMDKVFERLGVTAGCAEEGLQNKVLRKGCEKLGYKVESVSRNSSEGHYCGSCGYGCRSGDKRGTDRTWLVDAVSRGAVILTGCKAEKLLLERTGTGGAESRAKRCVGVVARSTNPTITRTLEVRARVTVSACGSLLTPVLLRGSGLSNRHIGKNLHLHPTALVWGYFPDTMPDLKGKTYEGGIITSLHKVEGAPGAPARAILETPAMGLAGAGTLFPWVSGSDLKERMLRYGRTVHLFSLVRDRGSGSVLGERRVAYHLDATDAENMREGLRRALRILAAAGAAEIGTHRSDGQRFACRGATEAALEEFLDGVDVVRGPQSKAEAWSLCCTAHQMGSCRMGVTARDGAVDARGESWEVESLYVCDGSVLPSAVGVNPMVTIQSVAYCLATGIAESLRRGPVPEKIQ

SEQ ID NO: 107 - DNA, 2277 bp, Arabidopsis thaliana
atggtaggaggaagaagattagggaagagaggaagccctttgttgagatggagtgtgaaacaagaaagcttcagccatggattctcaaaatctgacctccaagctctctcttccatttgcgacgctattatgcctcctgttccattagagagcttaaatcttgagatgaagttgaaggttttgcgcaacgatgcactcttatctttcttcaagtcttcttcttctgagtctcatgttcgaccagatgaggtggcagagctactggcgaccaaggcaataccattgacagttttagtggtgagaatagttttgagaatactcacattcagactggggacgttactgctttgcgggttagtctgtcttgacaagaagcattggccttttcttctcaagttctctgaaatgtccctggagaagagggaaaaggttcttcagagatggaacacacagtggtataacccactagcgagaatcggctttatgatgatcaaagccatcttcttgttctactacttcacatggacaaatgaaaattcagaaaacccagtatgggatgcaattaattatagcgtggaaattggtgaaaatgaggacatggaacaaaaggaaagacctctagatgaagggatcatcgagactgcaaaagaagatgagatgaccatcaagcaacgtatgatcaacaaaggcctcaaagtcacagaagacagagaaagggacacttacaagatcgagtgtgatgcagtggtagtgggttctggctgtggcggaggggttgcagctgcaattctagcaaagtcgggccttagggtggtagttatcgagaaggggaactactttgcccccagagactactcagctctcgagggcccttccatgtttgagctgtttgagtccaatagcttgatgatgactcatgatggcaggttccggttcatggcaggatcaacagtcggtggtggctctgtggtaaactgggcagcgtccttaaaaacacctgatgctatcatcgaagaatggtcagtacatcgagggatttcaatatattctagtgagaaatataaggcggcgatgggtattgtctgcaagaggttaggtgtcactgagaaaatcatcagagaagggtttcagaaccagattctgcggaaaggttgcgagaagcttggtttggatgtgacaatagtgccaaggaattcaacagagaaacattattgcggtagttgctcgtatggatgcccaaccggagagaagagagggacagattcaacctggctggttgatgcagttaacaacaatgctgtgatcctaacacagtgcaaggctgagaaactaatcttggcggataacgatgctaacaaaagagaggagagcggacgaagaaaaagatgtttgggagtcgcagcctctctatcccaccaaaccagaaagaagcttcagatcaatgccaaagtgacaattgtggcttgcggctcgcttaagacaccaggattgttggcttcaagcgggttgaagaattcaaacatcagtcgcggtctccatatccaccctatcatgatggcttggggctacttcccagaaaagaattcagaattggaaggagcggctcatgagggagagatcgtcacttcattgcattatgtgcatccaatggactccactacacctaacatcacactggagactcctgctataggaccaggtacgttcgcagctctgaccccgtgggtctctgggtcagacatgaaagaaagaatggccaaatacgcaagaacagctcatatttttgccatggtgagagatgaaggagttggagaggtgaaaggagacattgtcaaatacagattaaccaaagcggacgaagagaatctgacaattgggttaaagcaagcactgaggatcctagtagcagcaggagcagcggaggtaggcacatacaggagcgacgggcagagaatgaagtgtgatggaatcaaacagaaagatctagaggcatttttagataccgtaaacgcgccacctggagttgtgtccatgagtaagcattggactcaatcctttacagcgcatcaaataggatgttgccgtatgggtgccacagaaaaggaaggagccattgatggaaaaggagagagctgggaggcagaagatttgtatgtctgtgatgcaagtgttctgcctacagctcttggtgttaatcccatgatcaccgttcagtccactgcttactgcatatccaacagaatagccgagttaatgaagaagaggaagaaagactga

SEQ ID NO: 108 - protein, 758 aa, Arabidopsis thaliana
MVGGRRLGKRGSPLLRWSVKQESFSHGFSKSDLQALSSICDAIMPPVPLESLNLEMKLKVLRNDALLSFFKSSSSESHVRPDEVAELLATKAIPLTVLVVRIVLRILTFRLGTLLLCGLVCLDKKHWPFLLKFSEMSLEKREKVLQRWNTQWYNPLARIGFMMIKAIFLFYYFTWTNENSENPVWDAINYSVEIGENEDMEQKERPLDEGIIETAKEDEMTIKQRMINKGLKVTEDRERDTYKIECDAVVVGSGCGGGVAAAILAKSGLRVVVIEKGNYFAPRDYSALEGPSMFELFESNSLMMTHDGRFRFMAGSTVGGGSVVNWAASLKTPDAIIEEWSVHRGISIYSSEKYKAAMGIVCKRLGVTEKIIREGFQNQILRKGCEKLGLDVTIVPRNSTEKHYCGSCSYGCPTGEKRGTDSTWLVDAVNNNAVILTQCKAEKLILADNDANKREESGRRKRCLGVAASLSHQTRKKLQINAKVTIVACGSLKTPGLLASSGLKNSNISRGLHIHPIMMAWGYFPEKNSELEGAAHEGEIVTSLHYVHPMDSTTPNITLETPAIGPGTFAALTPWVSGSDMKERMAKYARTAHIFAMVRDEGVGEVKGDIVKYRLTKADEENLTIGLKQALRILVAAGAAEVGTYRSDGQRMKCDGIKQKDLEAFLDTVNAPPGVVSMSKHWTQSFTAHQIGCCRMGATEKEGAIDGKGESWEAEDLYVCDASVLPTALGVNPMITVQSTAYCISNRIAELMKKRKKD

SEQ ID NO: 109 - DNA, 2256 bp, Glycine max
atgaccagaaaagagtgtcatccattgttaaggggtgggagaggggatagcaaatataaacatggattttctgcagctgagatggagtcactggcaagcatatgtgaggttgtgttgcctcctttgcctatggatgctcttaagatcagaaaggaagaccaaattgatgattatgattctagcaagagtctcaagtccttctgggacatttctgcttctcgctatccaatccctcatgaggttgctgagatgttaacgaagaggagcttaattgaagcagtaatactgattagagtagttttatggctgctggcaacaaggttgggaaccttgttgctctgtggtttcctctgtcttggtgaaaaatggccctatgtcaacaacttctcaaacatatctttggagaaaagagaaatggttatgcagaagtggctgaaacataggttcctcacacctattagactggcctttgcttacatcaaagtcttgtgtctctttgtttttttctcttgggttgatgaaaatggtgacaacccagcatggaaagccattggatatgaggtaccagctgatgaaaacttgaccaatgcctccaaaactaggccccttgaaaaggggattatagaaaccatgaatgaatctgactccgctcttcaacaatctcttgctaacaaaggccttaatgttacactggactcaaaaagcaacatcctcaaagtcaaatgtgatgcactagttgttggttctggttgtggaggaggtgttgcggctgctgttctttcaagtgctggctacaaggtggttgttcttgagaaaggaaactatttttctactcaggattattcatctctagaaggtccttccatgaatcaactatatgaaactggagggatccttgcttctgtggactcaagagtgctagttttggcaggatcaacagtgggtggtggctctgctgttaattggtcagcctgcattaagaccccacacaaggtgctaaatgagtggtctgagaatcacaagcttcccttcttttcaagccaagaatatctctccgcaatggaaactgtgtgtgaaaggattggtgtaacagaaaactgtacacaagagggattccaaaaccaagtgctgagaaaagggtgtcaaaatcttggcctcaaagttgactatgtgccaagaaactcctcagggaatcactactgtggctcatgtggttatggctgtccgaaaggagagaaacaagggactcaagctacatggcttgtagatgcagttgaaagagatgcagtaataataacaggatgcaaagctgagaggttcttgttggaaagcaacaggagtggaaatggcagaaagaagaaatgtttgggagtgatggcaaaagcattaaacagcagagtcacaatgaagttacaaattgaggccaaggtgacaatttctgcaggtggggcacttttgacaccccctttgttaatctctagtggactaaaaaacaagaacataggtaaaaaccttcatctccaccctgtgctaatgacatggggatactttccagaatcaaatgattcagaattcaaaggaaaagtctacgagggaggtataataacatcagtccacaaagtgccatcaacagactccaattcagattcaagggcaataattgaaaccccttcgctaggaccagcttcctttgcagcactgtgtccttgggagtcaggactagactttaaggaaagaatgctgaattaccctagaacttcacatttgataacaataataagagacatggcttgtggacaggtatcaacagaaggaaggatcagctacaagctgaatgaaattgacaaggagaacatgaaggctggcttgaaacaagcactgaagattcttatagctgcaggagcagttgaggtaggtacacacagaagtgatggtcagagacttaagtgtgatggcattggtgaaaatgaagtgcaagagttcttggacagtgtgtgtccaatggagggggcactttcaccaggtgagtattggaacatatattcttctgcacatcaaatggggagctgcagaatgggagtcaatgagaaagaaggtgcagttgatgaaaatggtgagacatgggaggctgaagggttgtttgtttgtgatgctagtgtgcttccaagtgctgttggtgtcaatcccatgatcacggtccaatcaactgcatactgcatctctaatagaatagcagattatcttagaagggattaa

SEQ ID NO: 110 - protein, 751 aa, Glycine max
MTRKECHPLLRGGRGDSKYKHGFSAAEMESLASICEVVLPPLPMDALKIRKEDQIDDYDSSKSLKSFWDISASRYPIPHEVAEMLTKRSLIEAVILIRVVLWLLATRLGTLLLCGFLCLGEKWPYVNNFSNISLEKREMVMQKWLKHRFLTPIRLAFAYIKVLCLFVFFSWVDENGDNPAWKAIGYEVPADENLTNASKTRPLEKGIIETMNESDSALQQSLANKGLNVTLDSKSNILKVKCDALVVGSGCGGGVAAAVLSSAGYKVVVLEKGNYFSTQDYSSLEGPSMNQLYETGGILASVDSRVLVLAGSTVGGGSAVNWSACIKTPHKVLNEWSENHKLPFFSSQEYLSAMETVCERIGVTENCTQEGFQNQVLRKGCQNLGLKVDYVPRNSSGNHYCGSCGYGCPKGEKQGTQATWLVDAVERDAVIITGCKAERFLLESNRSGNGRKKKCLGVMAKALNSRVTMKLQIEAKVTISAGGALLTPPLLISSGLKNKNIGKNLHLHPVLMTWGYFPESNDSEFKGKVYEGGIITSVHKVPSTDSNSDSRAIIETPSLGPASFAALCPWESGLDFKERMLNYPRTSHLITIIRDMACGQVSTEGRISYKLNEIDKENMKAGLKQALKILIAAGAVEVGTHRSDGQRLKCDGIGENEVQEFLDSVCPMEGALSPGEYWNIYSSAHQMGSCRMGVNEKEGAVDENGETWEAEGLFVCDASVLPSAVGVNPMITVQSTAYCISNRIADYLRRD

SEQ ID NO: 111 - DNA, 315 bp, Oryza sativa
atggccctcgcgtgtgcgtctcatttgcgccgcctcggagccggagctccggcgagatccttccacgcgcatccgtaccaagccaaggtcggcgtggtggagttcctgaacggggtcgggaagggggtggagacgcacgccgccaaggtggaggaggcggtcggcggcgacctccagagcctcctccacgcccgcactctgcggctcaagaagctcggcatcccctgcaagcagaggaaattaattttgagtttcgctcacaagtatcgtcttggtctttggaagccccaagcagaatccaagaaaacacagtga

SEQ ID NO: 112 - protein, 104 aa, Oryza sativa
MALACASHLRRLGAGAPARSFHAHPYQAKVGVVEFLNGVGKGVETHAAKVEEAVGGDLQSLLHARTLRLKKLGIPCKQRKLILSFAHKYRLGLWKPQAESKKTQ

SEQ ID NO: 113 - DNA, 312 bp, Zea mays
atggctctcgcgtgcgcctctcacgcgtgccgctttctcctcgccggccaggcgagatctttccacgcccagccctaccatgccaaggtcggcgtggtggagttcctgaacggcgtcgggaaaggggtggaggcgcacgccgcgaagctggaggaggctgtgggcggcgacctccagaggctccttgaggcccgcacgctgcggctcaagaagctcggcatcccctgcaagcatagaaaattgattttgagttttgcacacaagtaccgtcttggtctctggaagccccaagcagaacccaggaaagtttaa

SEQ ID NO: 114 - protein, 103 aa, Zea mays
MALACASHACRFLLAGQARSFHAQPYHAKVGVVEFLNGVGKGVEAHAAKLEEAVGGDLQRLLEARTLRLKKLGIPCKHRKLILSFAHKYRLGLWKPQAEPRKV

SEQ ID NO: 115 - DNA, 315 bp, Sorghum bicolor
atggctctcgcgtgcgccactcacgcgcgccgcttcctcctcgccggtccggcgagatctttccacgcccagccctaccaagccaaggtcggcgtggtggagttcctgaacggcgtcggcaagggggtggaggcgcacgccgcgaagctggaggatgctgtgggcggcgacctccagaggctcctcgagacccgcacgctgcggctcaagaagctcggcatcccctgcaaacatagaaagttgattttgagttttgctcataagtaccgtcttggtctctggaagccccaggcagatcccaggaaagttgaataa

SEQ ID NO: 116 - protein, 104 aa, Sorghum bicolor
MALACATHARRFLLAGPARSFHAQPYQAKVGVVEFLNGVGKGVEAHAAKLEDAVGGDLQRLLETRTLRLKKLGIPCKHRKLILSFAHKYRLGLWKPQADPRKVE

SEQ ID NO: 117 - DNA, 339 bp, Arabidopsis thaliana
atggcgtggatgcgactgatacagagcgcgagatctgttcttacgacagcaccagcgtcttcgactcttaacttccatagattctactccaaatccgccgctccttatcacgtgaaagttggaattcctgagtttttgagtgggattggtggaggagttgagactcatattgctaagcttgaaaccgagttaggcgatcttccgaaattgctcgtgactcgtacgcttagactcaagaagtttggtattccttgcaaacataggaaattgatactgaaatatagccacaaatacaggctaggtctatggaaacctagagctgacgcaataaaggcgtga

SEQ ID NO: 118 - protein, 112 aa, Arabidopsis thaliana
MAWMRLIQSARSVLTTAPASSTLNFHRFYSKSAAPYHVKVGIPEFLSGIGGGVETHIAKLETELGDLPKLLVTRTLRLKKFGIPCKHRKLILKYSHKYRLGLWKPRADAIKA

SEQ ID NO: 119 - DNA, 375 bp, Glycine max
atggacggtgcgagtgggagaggcagcaagaagagaaaaaggggaatggcatggttgcagttgcagagaatcatcaccaataccaagggaggtgctaatttgatctcaccgctcccacgattcttctcgaaatgttccccctatgttttgaaagttggaataccagagtttctaaacgggattgggaagggggttgagtcccacgttcctaagcttgaatctgaaatcggtgatttccagaaacttctcgtcattcgaacccttaaactcaagaagctcggtattccttgcaaacataggaagttaatattgaaatacacacacaagtacaggctgggattatggaggccacgtgctgagttcatcactgcctga

SEQ ID NO: 120 - protein, 124 aa, Glycine max
MDGASGRGSKKRKRGMAWLQLQRIITNTKGGANLISPLPRFFSKCSPYVLKVGIPEFLNGIGKGVESHVPKLESEIGDFQKLLVIRTLKLKKLGIPCKHRKLILKYTHKYRLGLWRPRAEFITA

SEQ ID NO: 121 - DNA, 1215 bp, Oryza sativa
atggtggtgtcgcgggcgcgggtgcaggacccggcgaccggcgagcaccgcgacgtgatgtacaagggcatggcgtcggagctgttcgtgccgtacatggacccgacggaggcgtggtacttcaagacgtacatggacgccggcgagtacggcttcgggctgcaggccatgccgctcgtcccgctcaacgactgcccgcgccacgcccgctacctcgacgccgtcttcgtcgccgccgacggccgcccctacgtgcgcgagaacatgatctgcgtcttcgagcgctacgccggcgacatcgcgtggcgacactccgagagccccatcaccggcatggacataagggagtcgcggccgaaggtgacgctggtggcgcgcatggcggcgtcggtggccaactacgactatatcgtcgactgggagttccagatggacggcctcgttcgcatcaaggttgggctaagtgggatcctcatggtgaagggcacccagtactcccacatgaaccaggtccatcaaaatgacaatatgtacggcacccttctgtctgagaacgttattggcgtcatccatgaccactttgtcactttccggctcgacatggacattgacggcgccgacaactccttcgtcaaggtggcgatggcacgacagaacaccggcgccggcgaatcccctcgtaagagctacctgaaggctacccgacatgtcgcaaggacggagaaagatgcccaggtccgcctgaagctatatgagccatccgagttccacattgtcaaccccatgaagaagacacgagttgggaaccctgttggttataaggttgtcccagccggtactgcagctagcctgttggatccagaggacccacctcagaagaggggtgcattcacaaataatcagatttgggtgacaccctacaacaaaacagaggaatgggctggtggtctatttgtctaccagagcaaaggggaggacacacttgcaacttggtccgaaagggaccgtccgatcgagaacaaggacctggtgctgtggtacacgctggggtttcaccatgtcccgtgccaggaggacttccccatcatgcccacggtgtcgtccagcttcgaccttaagccagtgaacttctttgagagcaaccccatcctagggcagcgtccgacccaggagaacgacctgccggtgtgtgccgccgctgccacgacctcttga

SEQ ID NO: 122 - protein, 404 aa, Oryza sativa
MVVSRARVQDPATGEHRDVMYKGMASELFVPYMDPTEAWYFKTYMDAGEYGFGLQAMPLVPLNDCPRHARYLDAVFVAADGRPYVRENMICVFERYAGDIAWRHSESPITGMDIRESRPKVTLVARMAASVANYDYIVDWEFQMDGLVRIKVGLSGILMVKGTQYSHMNQVHQNDNMYGTLLSENVIGVIHDHFVTFRLDMDIDGADNSFVKVAMARQNTGAGESPRKSYLKATRHVARTEKDAQVRLKLYEPSEFHIVNPMKKTRVGNPVGYKVVPAGTAASLLDPEDPPQKRGAFTNNQIWVTPYNKTEEWAGGLFVYQSKGEDTLATWSERDRPIENKDLVLWYTLGFHHVPCQEDFPIMPTVSSSFDLKPVNFFESNPILGQRPTQENDLPVCAAAATTS

SEQ ID NO: 123 - DNA, 2235 bp, Zea mays
atggatcgctccacctccctgctccggctcatcttgcttgctcttggcgcagccctggtgctcctcgtcgtccgctccgccttccgcctcccacgtggaatcgacacacccaccacctcactcttcgatgacgaagccaccgcgggcagcagctgcacccggttcgcgccgtgggggtgccggcaagccgaccggattaagcagaagccgccgtcgcacgagaacgacgtgccgcggcacccgctcgacccactgacgatcagcgaggtaaaccgtgcgcgcgagctcctccgcgcgcacccgccgttcgcatcgtcgccgtcgtccatgttcgtgcactcgctcgcgctcgacgagccggacaagcccgtcgtcctgagctggcggaagggcgtcgacccgctgcccccgcggcgcgcggtggcggtggtccggttccgcggtgaggccttcgtcctggccattgacctcgccagcggcgccgtgactcctctgcctgtcccagcttccgggtacccgaccatgaccatggacgagcaggtgctcctctgctacacgcctttcagggacccggcgttcaacgcgaccatccagcggcacggcgtccgcctgtccgatgtcgcctgcctgcccatctcgctcgggtggtacggccccagcgaggagaaccggcggctgatcaagatccagtgcttctccgcggagggcaccgccaacttctacatgcgccccatcgagggcctcaccgtgctggtggacatggacacgagggaggtggtccgcatctccgaccgcggcgccggcatccccatcccgcccgccgccaacaccgactaccggtactcccgccacatgcaagacgaaggcgacgaccagcagacagcggggttccagaaggtgcgggcgccgtcgatggaaccggggccgtcgtcagggccgcgggtggagctggtggacggccacacggtgcggtggggcgggtgggagttccacctcaaggcggacgcgcgcgccggcatggtggtgtcgcgcgcacgggtgcaggaccccggcacgggcgcgcaccgggaggtgctgtacaagggcatggcgtcggagctgttcgtaccgtacatggaccccaccgaggcgtggtacttcaagacgtacatggacgccggcgagtacggcttcggcctgcaggccatgccgctggtgccgctcaacgactgcccgcgccacgcgcgctacctggacggcgtgttcgtggccgccgacggacggccctacgtgcgcgagaagatgatctgcgtcttcgagcggtacgccggtgaggtcgcgtggagacactcggagagccccatcaccggcttggacataagggagtcgcggccgaaggtgacgctggtggcgcggatggttgcgtccgtggccaactacgactacatcatggattgggagttccagatggacggtctcgtccgcattaaggttgggctgagcgggatcctgatggtgaagggcacgtcctactcccacctgagcgaggcccgcgggaacgagggcgacatgcacggcacgctgctctcggagaacgtgatcggcgtcatccacgaccactacgtgaccttccgcctggacatggacgtcgacggcgccgacaactcgttcgttcgcgtggagatggcgcggcaggagacgggccccggcgatgagtcgccccggaggagctacctcagggccacccgccgcgtggcggagaccgagaaggacgcgcgggtgcgcctcagcctctaccacccggcggagttccacgtcgtcaaccccgccaagaagacgcgcgtcggcaaccccgttggctacaaggtcgtccctgccggcaccgccgcgagcttgctggacccggaggatccgccgcagaagaggggtgctttcacaaacaatcagatatgggtgacgccctacaacaagagcgaggaatgggccggcggcctgttcgtgtaccagagcaaaggggaggacactctggatacttggtccgagagagaccgtccgatcgagaacaaggacctggtgctgtggtacacgctggggttccaccacatcccgtgccaggaggacttccccatcatgcccaccgtgtcgtcaagcttcgacctcaagccagtcaacttcttcgagagcaaccccatcctcaagcagcgcccgaccaaagaggatgatctgcccatctgtactgcctccaccgccgcgtag

SEQ ID NO: 124 - protein, 744 aa, Zea mays
MDRSTSLLRLILLALGAALVLLVVRSAFRLPRGIDTPTTSLFDDEATAGSSCTRFAPWGCRQADRIKQKPPSHENDVPRHPLDPLTISEVNRARELLRAHPPFASSPSSMFVHSLALDEPDKPVVLSWRKGVDPLPPRRAVAVVRFRGEAFVLAIDLASGAVTPLPVPASGYPTMTMDEQVLLCYTPFRDPAFNATIQRHGVRLSDVACLPISLGWYGPSEENRRLIKIQCFSAEGTANFYMRPIEGLTVLVDMDTREVVRISDRGAGIPIPPAANTDYRYSRHMQDEGDDQQTAGFQKVRAPSMEPGPSSGPRVELVDGHTVRWGGWEFHLKADARAGMVVSRARVQDPGTGAHREVLYKGMASELFVPYMDPTEAWYFKTYMDAGEYGFGLQAMPLVPLNDCPRHARYLDGVFVAADGRPYVREKMICVFERYAGEVAWRHSESPITGLDIRESRPKVTLVARMVASVANYDYIMDWEFQMDGLVRIKVGLSGILMVKGTSYSHLSEARGNEGDMHGTLLSENVIGVIHDHYVTFRLDMDVDGADNSFVRVEMARQETGPGDESPRRSYLRATRRVAETEKDARVRLSLYHPAEFHVVNPAKKTRVGNPVGYKVVPAGTAASLLDPEDPPQKRGAFTNNQIWVTPYNKSEEWAGGLFVYQSKGEDTLDTWSERDRPIENKDLVLWYTLGFHHIPCQEDFPIMPTVSSSFDLKPVNFFESNPILKQRPTKEDDLPICTASTAA

SEQ ID NO: 125 - DNA, 2259 bp, Sorghum bicolor
atggatcactccacctccctgctccggctcatcttccttgctcttggcgcagccctggttctccttatcgtccgctccgccttccgcctcccacgtggaatcgacacacccaccacctcactcttcgatgacgccaccgccggcagcagctgcacacggttcgcgccatgggggtgccgccaggccgaccggattaagcaggagcagaagccgaagccggagccgccgtcgcacgagaacgacgtgccgctgcacccgctcgacccactgacggtcaccgagataaaccgtgcgcgcgagctcctccgcgcgcacccgccgttcgcgtcgtcgccgtcgtccatgttcgtgcactcgctggcgctcgacgagccggacaagcccgtcgtcctgagctggcggaagggcgccgacccgctgcccccgcgacgcgccgtggcggtggtccggttccgcggcgaggccttcgtcctcgccatcgacctcgccagcggcgccgtgactcctctgcctgccccagcttccgggtacccgaccatgaccatggacgagcaggtgtccctctgctatgcccctttcagtgacccggcgttcaacgccaccgtccagcggcacggcgtccgtatgtccgacgtcgcctgcctgccgatctccctcgggtggtacggccccaccgaggagaaccgccggctgatcaagatccagtgcttctccgcggagggcacggccaacttctacatgcgccccatcgagggcctcaccgtgctgctggacatggacacgagggaggtcatccgcatctccgaccgcggcggcggcatccccatcccgcccgccgccaacaccgactacaggtatgcccgccacatgcaagaagacgtcggcggcgaccagacgacgtcgaggagtgaggcggggttccagaaggtgcgggcgccgtcgatggagccggggccgtcgtcggggccgggagtggagctggtggacgggcacacggttcggtggggcgggtgggagttccacctcaaggcggacgcgcgcgccggcatggtggtgtcgcgcgcacgggtgcaggaccccggcacgggcgcgcaccgggaggtgctgtacaagggcatggcgtcggagctcttcgtgccgtacatggacccgaccgaggcgtggtacttcaagacgtacatggatgccggcgagtacggcttcggcctgcaggccatgccgctggtgccgctcaacgactgcccacgccacgcacggtacctcgacggcgtgttcgtggccgccgacggccggccctacgtgcgggagaagatgatctgcgtcttcgagcggtacgccggcgaggtcgcgtggagacactcggagagccccatcaccggcatggacataagggagtcgcggccgaaggtgacgctggtggcgcggatggttgcgtccgtggccaactacgactacatcatggactgggagttccagatggacggcctcgtccgcatcaaggtgggcctgagcgggatcctgatggtgaagggcacggcctactcccacctacgggaggcccgcgagaacgaggacacgcacggcacgctgctctccgagaacgtcatcggcgtcatccacgaccactacgtgacgttccgcctggacatggacgtcgacggcgccgacaacaactccttcgtgcgcgtggagatggcgcggcaggagacggcccccggcgagtccccccggaggagctacctcaaggccacccggcacgtggcgcggaccgagaaggacgcccaggtgcgcctcaagctctacgatccggccgagttccatgtcgtcaacccgaccaagaagacgcgggtcggcaaccccgttggctacaagctcgtccctgccggcaccgccgccagcttgctggacccggaggatccgccgcagaagaggggtgctttcacaaacaatcagatctgggtgacaccctacaacaagagcgaggaatgggccggtggtctgttcgtgtaccagagcaaaggggaggacacattggctacttggtccgagagagaccgtccgatcgagaacaaggacctggtgctgtggtacacgctggggttccaccacatcccgtgccaggaggacttccccatcatgcccaccgtgtcctcaagcttcgacctcaagcctgtcaacttcttcgagagcaaccccatcctcaagcagaggcccaccaaggaggatgatctgcccatctgttccgccaccgccgtgtaa

SEQ ID NO: 126 - protein, 752 aa, Sorghum bicolor
MDHSTSLLRLIFLALGAALVLLIVRSAFRLPRGIDTPTTSLFDDATAGSSCTRFAPWGCRQADRIKQEQKPKPEPPSHENDVPLHPLDPLTVTEINRARELLRAHPPFASSPSSMFVHSLALDEPDKPVVLSWRKGADPLPPRRAVAVVRFRGEAFVLAIDLASGAVTPLPAPASGYPTMTMDEQVSLCYAPFSDPAFNATVQRHGVRMSDVACLPISLGWYGPTEENRRLIKIQCFSAEGTANFYMRPIEGLTVLLDMDTREVIRISDRGGGIPIPPAANTDYRYARHMQEDVGGDQTTSRSEAGFQKVRAPSMEPGPSSGPGVELVDGHTVRWGGWEFHLKADARAGMVVSRARVQDPGTGAHREVLYKGMASELFVPYMDPTEAWYFKTYMDAGEYGFGLQAMPLVPLNDCPRHARYLDGVFVAADGRPYVREKMICVFERYAGEVAWRHSESPITGMDIRESRPKVTLVARMVASVANYDYIMDWEFQMDGLVRIKVGLSGILMVKGTAYSHLREARENEDTHGTLLSENVIGVIHDHYVTFRLDMDVDGADNNSFVRVEMARQETAPGESPRRSYLKATRHVARTEKDAQVRLKLYDPAEFHVVNPTKKTRVGNPVGYKLVPAGTAASLLDPEDPPQKRGAFTNNQIWVTPYNKSEEWAGGLFVYQSKGEDTLATWSERDRPIENKDLVLWYTLGFHHIPCQEDFPIMPTVSSSFDLKPVNFFESNPILKQRPTKEDDLPICSATAV

SEQ ID NO: 127 - DNA, 2277 bp, Oryza sativa
atggcttccctcctcacgctcccctccctctccctctccaaccctagcgcctccgccgccgccgcgggagccggagcggcgccgtctctgcgcctccgcgccgccttccgctgctgggcgctgcggcgcgcgggcggcggccggtgggcggccgcgggggccatcgcgtcgcccaactcggtgctcagcgagcacgcgttcaagcggctgcagctcagcgacgaggaggaggaggaggaggagggggcctacgggagtgacgaggagggggtcgaggcggtgggtggcggggagggggacgaggacgagctcgccattgccaggctcggcctgcccgagcagctcgtatccacgctcgagaagcgcgggattacccacctgttccccatccagagggctgtattgattccagctcttgacggccgtgacctgattgctagagcaaagacaggaaccgggaagacgctagccttcggcattcccatgatcaagcaattaatggaggaggatgatggacggagtgtgaggcgaggtcgtattcctcgtgttttggttctagcacctactagggagttagccaagcaagttgagaaggaaataaaagaatcagcacccaagctcagtacagtctgtgtttatggaggtgtctcatataatgtccagcagaacgcactctcccgtggtgttgatgttgttgtaggaacacctggtcgcattattgatttgataaatggcggaagtcttcagttgggagaagttaagtacttggtccttgatgaggctgaccaaatgcttgcagttgggtttgaggaggatgtggaaacgatattacaacagctaccagctgagaggcaaagcatgctcttctctgccaccatgcctggttgggtgaagaaattatctaggcgatacttgaacaatcctttgacaattgacttggtcggcgatcaagatgaaaaacttgctgaaggaatcaaactttatgctatcccgctcacatctacatcgaagcgcactgttcttagtgatctcattacggtgtatgcaaagggtgggaaaaccattgtgttcaccaagacgaaaagggatgcagacgaggtatcattagcactgacaaacagtattgcttcggaggcactgcatggtgatatttcacagcatcagcgtgagaggacactaaatggttttcgtcaagggaaatttactgttctcgtagcaaccgatgttgctgcccgtggtcttgatataccgaatgttgatttgattatccattatgaattgcccaatgatccagagacttttgttcatcgttctggacgcactggccgagctgggaaagcaggaactgcaatcttgatgttcacaaacagccagagaaggacagttagatcacttgagcgtgatgttggatgcagatttgacttcataagtcctccagcaattgaggatgtgctggagtcctctgctgaacatgtcatagctactttaagaggtgtgcacacggagtcgattcagtacttcattccagcagctgaaagactgcaagaagaattaggacctaatgctcttgcttctgcattggcacatctgagtggattttctcagccaccctcttcacgttctcttatcagtcatgagcagggatgggtgactttgcaactaactagagatccaggatatggaagggggttcttttctcctagatctgttactggctttctgtctgatgtttcttcagctgctgctgatgaagttggcaaaattttccttacagcagatgagaaggttcaaggtgcagttttcgatttacctgaggagattgcgagggatttgcttagtatggaactgcccccaggaaacaccataaccaaagtgacaaagctacctgcattacaggatgatggtcctgctactgatagttatggccgattctcaaactcggaccggggtttcaggaacagacggtccaggggtggtggctcaagaggcgggcggggtggttgggactctgatggtgaagacagatttcgccgtggtggcaggagcttcagatctgacaatgatagttggtcagatgatgactttggtggcgggagaagatcgaaccgttcgtcatcctttggtggccgcgggtcatcttacggcagtcgtagctcgtcatcctttggtggccgctcatcatcttttgggtctagggacagcagcaggagcttcagtggtgcttgcttcaactgtggcgaaagcgggcaccgagcatcagactgcccaaacaagtag

SEQ ID NO: 128 - protein, 758 aa, Oryza sativa
MASLLTLPSLSLSNPSASAAAAGAGAAPSLRLRAAFRCWALRRAGGGRWAAAGAIASPNSVLSEHAFKRLQLSDEEEEEEEGAYGSDEEGVEAVGGGEGDEDELAIARLGLPEQLVSTLEKRGITHLFPIQRAVLIPALDGRDLIARAKTGTGKTLAFGIPMIKQLMEEDDGRSVRRGRIPRVLVLAPTRELAKQVEKEIKESAPKLSTVCVYGGVSYNVQQNALSRGVDVVVGTPGRIIDLINGGSLQLGEVKYLVLDEADQMLAVGFEEDVETILQQLPAERQSMLFSATMPGWVKKLSRRYLNNPLTIDLVGDQDEKLAEGIKLYAIPLTSTSKRTVLSDLITVYAKGGKTIVFTKTKRDADEVSLALTNSIASEALHGDISQHQRERTLNGFRQGKFTVLVATDVAARGLDIPNVDLIIHYELPNDPETFVHRSGRTGRAGKAGTAILMFTNSQRRTVRSLERDVGCRFDFISPPAIEDVLESSAEHVIATLRGVHTESIQYFIPAAERLQEELGPNALASALAHLSGFSQPPSSRSLISHEQGWVTLQLTRDPGYGRGFFSPRSVTGFLSDVSSAAADEVGKIFLTADEKVQGAVFDLPEEIARDLLSMELPPGNTITKVTKLPALQDDGPATDSYGRFSNSDRGFRNRRSRGGGSRGGRGGWDSDGEDRFRRGGRSFRSDNDSWSDDDFGGGRRSNRSSSFGGRGSSYGSRSSSSFGGRSSSFGSRDSSRSFSGACFNCGESGHRASDCPNK

SEQ ID NO: 129 - DNA, 2115 bp, Zea mays
atgtctcccgccctcgccgctgccgtggagcccatggccgtcgacgactccgcctccaagaaggccaagcgcaagcagctcaaagccgctgccgccgccgccgccgcggaggcagaggaggaggctgcttcggccaagaagaaagagaagaaggagaagaagcggaaagcgaaggagccgtcccctccgctcccgtcggccgcatcatctggcgaggagaagagcagcaccagctccgaggagacagcccccaccgcgaagaaggcgaagaaggacaagacgaagaagaatgtcgaggtctcgtcttcagcctcagatgacgacggcgagatcacggccggcagcgacgaggaccccgcggacccgaacgcgctgacgaacttcaggatatcggagccactgaggcagagcctcaggtccaaggggatcaaggcactgttccccatccaggccaccactttcgacctcgtactcgacggcagcgacttggttggccgagcgcgcaccggtcagggaaaaactttggcttttgtcttgcccatattggaatctttggttaacggggcaaataaggcatctaggcggactgaacatggcaggaccccaagtgttctcgttctgctaccaacaagagagctggccaatcaggtgcatgccgactttgagttttatggtgcaacatttgggctttctgcatgttgtgtgtatgggggttcaccttatcgtcctcaagaaatggcattgagaagaggcgtggacattgttgttggaactcctggtcgtatcaaggattttattgtaaaaggaactctcaacttgaaatgcttgaagttccgtgtccttgacgaagctgatgagatgcttaacatgggctttgttgatgatgtcgagctcattcttggcaaggtagaagatgctaccaaagtacagacacttctgttcagtgccactctgccagattgggtgaataagctctctatgaggtttctgaaagttgacaggaaaacagttgatcttgtcggtaacgagaaactgaaggccagtgcatctgttaagcaccttgctcttccttgtaacaaggcagcaagggcacaacttattccagatattatccgatgttacagccatggaggccgaaccattatcttcactgagacgaaggattctgcatcagagctttctggtttgattcctggatcccgtgccttgcatggagatgttgtgcaagctcagcgtgaagtcattcttgctggatttcgtagcgggaagttccaggttttggttgctacaaatgtggcggctcgtggtctggatattaatgatgtgcagcttatcattcagtgtgaacctccccgtgacgttgaagcttacatacaccggtcaggtcggacagggagagcaggtaatactggtgttgctgtcatgctttatgagcccagatataagtacagtgtcagcagactagaaagggaatctggggttaagttcgaacatatctctgcaccacaacctactgatgtagcacaatctgctggcagtgaagctgcagatgccattgcgagtgtgtcagacagtgttattcctgtcttcaggcagcaagcagagcagttgctaagctcttccactctgtctgcggctgacttgcttgccaaagctcttgcaaaggcagttggttacacggacataaagaaaaggtcattgttgtcttcaatggaggattacgctacactacatcttcaaactggcagacagatgtggtcacctgggtttgcttttactatattgaaaaggttcatgccagaagagaaacttgcagatgtaaagggtgcaaccctcacggctgatggaacgggggttgtatttgatgttcctgcagcagatgttgaagattacattcaagcttcggagaatgccgcacaggtgacaattgatgaagtccagcaattgccacccttgcaagagaagcagcagcagcagtcaagaggcaactcggggggaggaagatttggccgtggaggtggcgggagattctctggtggtggccgtgggggcggcttcggtggtggcggaagaggcagaggtggtggtggcggcagggggaggcggcggcaatag

SEQ ID NO: 130 - protein, 704 aa, Zea mays
MSPALAAAVEPMAVDDSASKKAKRKQLKAAAAAAAAEAEEEAASAKKKEKKEKKRKAKEPSPPLPSAASSGEEKSSTSSEETAPTAKKAKKDKTKKNVEVSSSASDDDGEITAGSDEDPADPNALTNFRISEPLRQSLRSKGIKALFPIQATTFDLVLDGSDLVGRARTGQGKTLAFVLPILESLVNGANKASRRTEHGRTPSVLVLLPTRELANQVHADFEFYGATFGLSACCVYGGSPYRPQEMALRRGVDIVVGTPGRIKDFIVKGTLNLKCLKFRVLDEADEMLNMGFVDDVELILGKVEDATKVQTLLFSATLPDWVNKLSMRFLKVDRKTVDLVGNEKLKASASVKHLALPCNKAARAQLIPDIIRCYSHGGRTIIFTETKDSASELSGLIPGSRALHGDVVQAQREVILAGFRSGKFQVLVATNVAARGLDINDVQLIIQCEPPRDVEAYIHRSGRTGRAGNTGVAVMLYEPRYKYSVSRLERESGVKFEHISAPQPTDVAQSAGSEAADAIASVSDSVIPVFRQQAEQLLSSSTLSAADLLAKALAKAVGYTDIKKRSLLSSMEDYATLHLQTGRQMWSPGFAFTILKRFMPEEKLADVKGATLTADGTGVVFDVPAADVEDYIQASENAAQVTIDEVQQLPPLQEKQQQQSRGNSGGGRFGRGGGGRFSGGGRGGGFGGGGRGRGGGGGRGRRRQ

SEQ ID NO: 131 - DNA, 2136 bp, Sorghum bicolor
atgtctcccgcccttgcctctgccgtggagcccatggccgtcgacgactccgcctccaagaaggccaagcgcaagcagctcaaggccgccgccgccgccgcggaggcagaggcggaggccgcttcggctaagaagaaagagaagaaggagaagaagcgcaaggcgaaggagccgtcccctccgcccccgtcggcctcgtcctctgacgaggaggagaagagcagcaccagctccgaggagacggcccccgccgcgaagaaagcgaagaaggagaagacgaagaagaatgtcgaggtctcgtcttcagcctcggacgacgacggcgagatcacggccagcagcgacgaggaccccgcggacccgaacgcgctgacgaacttcaggatatcggagccgctgaggcagagcctcaggtccaaggggatcaaggcgctgttccccatccaggccaccactttcgacctcgtactcgacggcaacgacttggttggccgagcgcgcaccggtcaggggaaaactttggcttttgtcttgcccatattggagtctttggttaacggggcacacaaggcatctagacgaactgaacatggcaggaccccaagtgttattgttctgctacccacaagagagctggccaatcaggtgcatgccgactttgagttttatggtgcaacatttgggctttccgcatgttgtgtgtatgggggttctccttatcgtcctcaagaaatggctttgagaaggggtgtggacattgttgttggaactcctggtcgtgtcaaggatttcattgtaaaaggaactctcaatttgaaaaacttgaaattccgtgtccttgatgaagctgatgagatgcttaacatgggttttgttgatgatgtcgagctcattcttggcaaagtagaagatgctaccaaagtacagacacttctgttcagtgccactctgccagattgggtgaataagctctctatgaggtttctgaaagttgacaggaaaacggttgatcttgtcggtaatgagaaactgaaggccagtgcatctgttaagcaccttgctcttccttgtaacaaggcagcaagggcacaagttattccagacattatccgatgctatagccatggaggccgaaccattatcttcactgagacaaaggattctgcatcagagctttctggtttgattcctggatcccgtgccttgcatggagatgtcgtgcaagctcagcgtgaagtcattcttgctggattccgtggcgggaagttccaagttttggttgctacaaatgtggcggctcgtggtctggatattaatgatgtgcagcttatcattcagtgtgaacctccccgtgacgttgaagcttacatacaccggtcaggtcggacagggagagcaggtaatactggtgttgctgtcatgctttatgagcccagatataaatacagtgtcaacagactagaaagggagtctggggttaagtttgagcatatctctgcgccacagcctactgatgtagcacaatctgctggcagtgaagctgcagatgccatttcgagtgtgtcagatagtgttattcctgtcttcaggcagcaagcagagcagttgctaagctcttccagcctgtctgcagctgacttgcttgccaaggcacttgcaaaggcagttggttacacggacataaagaaaagatcattgttatcttccatggaggattacactacattacatcttcaaactggcagaccgatgtggtcacctgggtttgcttttactatattgaaaaggttcatgccagaagagaaacttgcagatgtaaagggtgcaaccctcacggctgatggaacaggtgttgtatttgatgttcctgcagcagatgttgaagattacattcaagcttcggagaatgctgcacaggtgacaattgatgaagtcaagcaactgccacccttgcaagagaaggagcagtcaagaggcaactctggcggaggaagatttggccgtggaggtggtgggagattctctggtggtggccgtggaggcggcttcggtggtggtggcagaggcagaggtggtggcggcagagggttttctgggaggggaggcggcggcaacagatttaacaggaggaattag

SEQ ID NO: 132 - protein, 711 aa, Sorghum bicolor
MSPALASAVEPMAVDDSASKKAKRKQLKAAAAAAEAEAEAASAKKKEKKEKKRKAKEPSPPPPSASSSDEEEKSSTSSEETAPAAKKAKKEKTKKNVEVSSSASDDDGEITASSDEDPADPNALTNFRISEPLRQSLRSKGIKALFPIQATTFDLVLDGNDLVGRARTGQGKTLAFVLPILESLVNGAHKASRRTEHGRTPSVIVLLPTRELANQVHADFEFYGATFGLSACCVYGGSPYRPQEMALRRGVDIVVGTPGRVKDFIVKGTLNLKNLKFRVLDEADEMLNMGFVDDVELILGKVEDATKVQTLLFSATLPDWVNKLSMRFLKVDRKTVDLVGNEKLKASASVKHLALPCNKAARAQVIPDIIRCYSHGGRTIIFTETKDSASELSGLIPGSRALHGDVVQAQREVILAGFRGGKFQVLVATNVAARGLDINDVQLIIQCEPPRDVEAYIHRSGRTGRAGNTGVAVMLYEPRYKYSVNRLERESGVKFEHISAPQPTDVAQSAGSEAADAISSVSDSVIPVFRQQAEQLLSSSSLSAADLLAKALAKAVGYTDIKKRSLLSSMEDYTTLHLQTGRPMWSPGFAFTILKRFMPEEKLADVKGATLTADGTGVVFDVPAADVEDYIQASENAAQVTIDEVKQLPPLQEKEQSRGNSGGGRFGRGGGGRFSGGGRGGGFGGGGRGRGGGGRGFSGRGGGGNRFNRRN

SEQ ID NO: 133 - DNA, 2016 bp, Arabidopsis thaliana
atgccttccctaatgttatctgataagaaagaggagaagaagatgaagaagaagatggctttggataccccagagcttgattctaagaaggggaagaaggagcagaagctgaaactatctgattcggatgaagaagagtcggagaagaagaagagtaagaagaaggataagaagcgtaaggcttccgaggaggaggatgaagtgaagagcgattcaagctcggagaagaagaagagcagcaagaaggttaagttgggcgttgaggatgttgaagttgataaccctaatgctgtttctaagtttcggatttctgcgcctttgagggagaagcttaaggcgaatggtattgaagctcttttcccgattcaggcttccacgtttgatatggttctcgatggtgctgatttagttggacgggctcgtactggtcagggtaaaacattggcttttgtgttgcctatattggaatctttggttaatggacctgccaaaagcaaaaggaagatgggatatggcaggtcaccaagtgttttggtccttttaccgaccagagaattggccaagcaggtggctgctgactttgatgcatatggaggatcacttgggttaagttcatgttgtctctatggaggtgatagctatccagttcaggagggtaaattaaagagaggtgttgacattgtagttggaacccctggtcgtattaaggatcatattgaaagacaaaaccttgatttcagctatttacaattccgtgttcttgatgaagctgatgaaatgttgaggatgggatttgttgaggatgttgaacttattttagggaaagtggaggattctactaaagtccagactcttctcttcagtgctactttgccatcatgggtgaaaaatatctctaacaggtttcttaaaagagaccagaagactattgatcttgttggtaatgataaaatgaaggccagtaatagtgttcgacacattgctattccttgtaataaggcagccatggctcggttgattcctgatattatcagttgctatagcagtggaggccaaactattattttcgctgaaactaaagttcaagtttctgagctttctggtttgttggatggatcaagagccttgcatggtgaaatacctcaatcacaacgtgaggttactcttgctggatttaggaatggcaagtttgcaacattggtggctacaaatgttgctgctcgtggtctagatatcaatgatgtgcagttaattatccagtgtgagcctccacgtgaagttgaagcatatattcatcgttcaggccgaacaggaagagctggcaacacaggagttgcggttacactctacgattctagaaagtcgagtgtatccaggatcgaaaaagaagctggtatcaaatttgagcaccttgctgcacctcaacctgatgaaattgccagatctggtggtatggaagctgctgagaaagttaaacaagtgtgtgacagtgtggttcctgcattccttgaagctgccaaggaattattagaaacttctggtttatcagctgaagtactcctcgcaaaagctcttgcaaaaactgcgggcttcactgagataaagaaaaggtcacttcttacatcaatggagaactatgtgacactacatcttgaagcagggaaaccaatatactcgccatcatttgtttatggactgctaaggagagttttaccggatgacaaggtggaaatgattgaagggttatctctaacagcagataaaacaggagctgtgttcgatgttaagcaatcagatctagacttgttcattgcaggagcacaaaagagtgctggaagtatgagtttggaagtggttaaggtgatgcctaaactccaggagagagaaccattgccacaaaaaagattcggtggtggtggcagaggaaatcggtttggtggtggtggaggaaatcggtttggcggtggtggtggcagaggaagaggaggcagtggtggtagaggccagagatattga

SEQ ID NO: 134 - protein, 671 aa, Arabidopsis thaliana
MPSLMLSDKKEEKKMKKKMALDTPELDSKKGKKEQKLKLSDSDEEESEKKKSKKKDKKRKASEEEDEVKSDSSSEKKKSSKKVKLGVEDVEVDNPNAVSKFRISAPLREKLKANGIEALFPIQASTFDMVLDGADLVGRARTGQGKTLAFVLPILESLVNGPAKSKRKMGYGRSPSVLVLLPTRELAKQVAADFDAYGGSLGLSSCCLYGGDSYPVQEGKLKRGVDIVVGTPGRIKDHIERQNLDFSYLQFRVLDEADEMLRMGFVEDVELILGKVEDSTKVQTLLFSATLPSWVKNISNRFLKRDQKTIDLVGNDKMKASNSVRHIAIPCNKAAMARLIPDIISCYSSGGQTIIFAETKVQVSELSGLLDGSRALHGEIPQSQREVTLAGFRNGKFATLVATNVAARGLDINDVQLIIQCEPPREVEAYIHRSGRTGRAGNTGVAVTLYDSRKSSVSRIEKEAGIKFEHLAAPQPDEIARSGGMEAAEKVKQVCDSVVPAFLEAAKELLETSGLSAEVLLAKALAKTAGFTEIKKRSLLTSMENYVTLHLEAGKPIYSPSFVYGLLRRVLPDDKVEMIEGLSLTADKTGAVFDVKQSDLDLFIAGAQKSAGSMSLEVVKVMPKLQEREPLPQKRFGGGGRGNRFGGGGGNRFGGGGGRGRGGSGGRGQRY

SEQ ID NO: 135 - DNA, 2226 bp, Glycine max
aacgtttccaccgcgaaaaccttggtcggcgcatactccttattcttctcctgcttctgcacctcactgctataccccctttcaagcaggagaaaatctctcactcctttctctttctctccacgccgccatcaccacacaaccatgccttcgctctctctctctgaccctgacacccccaaacctattgccaagaagaaaaccaaaacccagtccctcaccgatcccgacctcgatggggtttcggggaagaagacgaagaagcgcaaggcttcagatttagaacaagaggccatgccccccgccgcttacaacaacgatggggacgacgagaccagctccgacctcgtccaacccgaacctgcttccagagaagacgacaacaagaataagaagaaaaagaagaaaaaggttgttaagtccgaagaaaaggaacagcctttggtcacggaggctaatggagaaaagaaggaggatcccaacgcgctttccaacttcaggatttcggaacccttgaggcaaaaattgaaggagaagggtatcgaatcactgtttcccattcaggccatgaccttcgacaccgttctcgatggttctgatttggtcggtcgggctcgcaccggtcagggtaaaactctggcatttgtgttgcccatattagagtctttaataaatggtccaacaaaagcttcaagaaagactggcttcgggaggactccaagtgttcttgtgcttctacctactagggaattggcctgtcaggtgcatgctgattttgatgtttatggtggggcaatgggattgagttcatgttgtttatatggcggagctccgtatcaaggtcaggaaattaagcttaggagaggtgttgatattgtcattggcacaccaggtcgtgtgaaggatcatatcgagaaagggaatattgacctgagccaactaaagtttcgtgtccttgacgaagccgatgaaatgctgaggatgggttttgttgaagatgttgaaatgattctaggcaaggtagaaaatgttaataaagttcagacacttcttttcagtgctactttgccagactgggttaagcaaattgctgcaagatttttgaagccagataagaaaactgctgaccttgttggaaatacaaaaatgaaggccagcatcaatgttaggcatattgttcttccttgtactagttctgccagggcccaacttatcccagatattattcgctgttatagcagtggaggccggacaattgtttttaccgagacaaaagaatctgcttctcagcttgcagggatcttgactggagcaaaagctctccatggtgacatacagcaatcaacacgtgaggttacactgtctggctttaggtctgggaaattcatgacattagttgccacaaatgtggcagctcgaggtcttgatattaatgatgttcagttaattatacagtgtgaacccccacgggatgtagaagcctatatccatcgttctggacgcacaggaagagcaggtaatactggggttgctgttatgctttatgacccaaaaagatcgaacatatctagaatagagagagagtctggtgtaaaatttgaacacgtatctgctcctcagcctgatgatattgccaaagccgttagtggggaagctgctgaaatgattatccaagtgtctgatagtgtggttcctgcattcaagtctgctgctgaagatcttttgaacaattctggtttaccagtcattgaattactggcaaaggctcttgcaaaggctgttggttatactgaagtaaagcaaagatcacttctaacttccatggagaactatgttacattacttcttgagactggcaaaccaatctacacccaatcttttgcctatggagtcttgaggagatttttgcctgaagagaaggttgaggccgtgaaaggtctttcaatcactgctgatggaaatggcgttgtttttgatgtagcagctaaagatttagacatatatcttaacggtcaggaaaatgcctcgaatgtaagtttagagatagtgaaaacattgccacagttgcaacagatggagcaacaccaaagaggtggcagatttggtgatggtggtggtcgtggttaccggtttggtggaagaggtggaggaggcaggaatggtaggttctccaatggtggcggaagaggtggacgtggcggcaactggggtggaaagagatggtga

SEQ ID NO: 136 - protein, 741 aa, Glycine max
NVSTAKTLVGAYSLFFSCFCTSLLYPLSSRRKSLTPFSFSPRRHHHTTMPSLSLSDPDTPKPIAKKKTKTQSLTDPDLDGVSGKKTKKRKASDLEQEAMPPAAYNNDGDDETSSDLVQPEPASREDDNKNKKKKKKKVVKSEEKEQPLVTEANGEKKEDPNALSNFRISEPLRQKLKEKGIESLFPIQAMTFDTVLDGSDLVGRARTGQGKTLAFVLPILESLINGPTKASRKTGFGRTPSVLVLLPTRELACQVHADFDVYGGAMGLSSCCLYGGAPYQGQEIKLRRGVDIVIGTPGRVKDHIEKGNIDLSQLKFRVLDEADEMLRMGFVEDVEMILGKVENVNKVQTLLFSATLPDWVKQIAARFLKPDKKTADLVGNTKMKASINVRHIVLPCTSSARAQLIPDIIRCYSSGGRTIVFTETKESASQLAGILTGAKALHGDIQQSTREVTLSGFRSGKFMTLVATNVAARGLDINDVQLIIQCEPPRDVEAYIHRSGRTGRAGNTGVAVMLYDPKRSNISRIERESGVKFEHVSAPQPDDIAKAVSGEAAEMIIQVSDSVVPAFKSAAEDLLNNSGLPVIELLAKALAKAVGYTEVKQRSLLTSMENYVTLLLETGKPIYTQSFAYGVLRRFLPEEKVEAVKGLSITADGNGVVFDVAAKDLDIYLNGQENASNVSLEIVKTLPQLQQMEQHQRGGRFGDGGGRGYRFGGRGGGGRNGRFSNGGGRGGRGGNWGGKRW

SEQ ID NO: 137 - DNA, 522 bp, Oryza sativa
atgccgaagatggtggtggaggtggccgaccggtacacggcgaggagggacgggcggccgaacaactccggcgaggcggacggagaggttcgcggcgacgttccggcgaaaccgagggcaggccggggtggagggcgatgcggcgtcgccgagggaggagacggcgacgtcggccgacgctctggcgcggcgaccggggcagccggaggagatgtcgacgaggaggaaggtgaggccggcgcgggcgacggcgttccggcgaaattcgggcgaggaggaggtgatgccggggaagaaggcgatgccgcggtgtcgaaggaggagacggcggcgtcgatcggcgcaccggcgatgaacagcagccggctggagggagtacagtggcaccaatgccactggttgccggcgggggcaccgttccggcgagtttccggcgaggagacgctttatattgccctctcatgtgttacgtcacctaaaggactccgtgtcctgatcgagaacaacccacctggatattaa

SEQ ID NO: 138 - protein, 173 aa, Oryza sativa
MPKMVVEVADRYTARRDGRPNNSGEADGEVRGDVPAKPRAGRGGGRCGVAEGGDGDVGRRSGAATGAAGGDVDEEEGEAGAGDGVPAKFGRGGGDAGEEGDAAVSKEETAASIGAPAMNSSRLEGVQWHQCHWLPAGAPFRRVSGEETLYIALSCVTSPKGLRVLIENNPPGY

SEQ ID NO: 139 - DNA, 423 bp, Zea mays
atgcagatccgggtgcagtgcgcgtgcggcgagtcgtcgtgcccggaatgggccgtcgtggagctgcagggcgtggtgcagccgcaggcctccttcgccggcgacatccggggactccacatcggccgcctctgctcctccccttccccagcctccgcctcgtccaaggcagggttcaccttcacggtggggtaccacgagctcgccggcacaaaggtgacgctcaagaagcccctgctggtgctcaggaagaagaaggttaatgctggtgctcaggaagaagagccaccgacggcggcggaggtggagctggaggtgatcggcatcatccggcacaagatcctattcaaggaccgccccaaggccctcatctcaaagccgccaaccaaggagaagaaggccgtgcagccagcagcaaagtga

SEQ ID NO: 140 - protein, 140 aa, Zea mays
MQIRVQCACGESSCPEWAVVELQGVVQPQASFAGDIRGLHIGRLCSSPSPASASSKAGFTFTVGYHELAGTKVTLKKPLLVLRKKKVNAGAQEEEPPTAAEVELEVIGIIRHKILFKDRPKALISKPPTKEKKAVQPAAK

SEQ ID NO: 141 - DNA, 426 bp, Sorghum bicolor
atgcagatccgcgtgcagtgcgggtgcggcgagtcgtcgtgcccggagtgggccgtcgtggagctgcagggcgtggtgcagccgcaggcctccttcgccggcgacatccgcggactccacatcggccgcctctgctccgccccctccccgtcctcatcttccaagcaggcagggtacaccttcacggttgggtaccacgagctcgccgggaccaaggtgacgctcaagaagcccctgctggtgctccggaagaagaaggtgaatgctggttgtggggaacaagagccaccggcggcggcggaggaggtggagctggaggtgattggcatcatccggcacaagatcctcttcaaggaccgtcccaaggccctcatctcaaagccgccaaccaaggagaagaagaccgtgcagccagcagcaaagtga

SEQ ID NO: 142 - protein, 141 aa, Sorghum bicolor
MQIRVQCGCGESSCPEWAVVELQGVVQPQASFAGDIRGLHIGRLCSAPSPSSSSKQAGYTFTVGYHELAGTKVTLKKPLLVLRKKKVNAGCGEQEPPAAAEEVELEVIGIIRHKILFKDRPKALISKPPTKEKKTVQPAAK

SEQ ID NO: 143 - DNA, 438 bp, Arabidopsis thaliana
atggagattcgagtgaagtgtagatgtggagaagaagagtgtagcgaatgggcgatcgtagagcttcaaggagtcgtcgagactcaagcttccttccaaggttctattcagaatctcgagatcggtcgtctctgccactccgattcctcacagggaacatacacgttcacggtgggttaccatgaactcgtaggctctaaagtgactctaaagaagccattactggttttgaagaagcttcaatttgatgaagtttcagggaaagcaacggagcttgaagttgttgggattatacgaacaaagatcttgtttaaaaccagacctaagcctcttatttccggtaacaacaacctctctctgaacaaattgaaacctctctattgtttcattatctctgcttgtttcaataatgtagtctctgtttgtactattgaatga

SEQ ID NO: 144 - protein, 145 aa, Arabidopsis thaliana
MEIRVKCRCGEEECSEWAIVELQGVVETQASFQGSIQNLEIGRLCHSDSSQGTYTFTVGYHELVGSKVTLKKPLLVLKKLQFDEVSGKATELEVVGIIRTKILFKTRPKPLISGNNNLSLNKLKPLYCFIISACFNNVVSVCTIE

SEQ ID NO: 145 - DNA, 414 bp, Glycine max
atgcagattcgagttcgatgcaactgtggagaaggaagctgcgaggaatggggggttatagaacttcaaggagtggtggagccacagcctggattccacgattccctccaaaatcttcacatcggcactttgtgtcgtccttcttctcaggaagtctacaccttcactgttggataccatgaactgacggggtcaaaggttcccttaaagaagccaatggtggtgctcaagaaagtaaagcatcctgatggagaaagtggttgtaaggtggagctgcaagttgttggagtcattagacataagattctgttcaagaacagaccaaaggctctcatttctaagccacagataacatccagggaaagacaaaagcctatcatgttagggtcttctccttcaaatcaaactgcttaa

SEQ ID NO: 146 - protein, 137 aa, Glycine max
MQIRVRCNCGEGSCEEWGVIELQGVVEPQPGFHDSLQNLHIGTLCRPSSQEVYTFTVGYHELTGSKVPLKKPMVVLKKVKHPDGESGCKVELQVVGVIRHKILFKNRPKALISKPQITSRERQKPIMLGSSPSNQTA

SEQ ID NO: 147 - DNA, 1182 bp, Oryza sativa
atggctgtgcggctctgctgctgctgcttcctcctcttcgtcaccatcgctgccgccggtggctccctgacgcggacgaacgtcgcgacgctgccggggttcgacggcgcgctgccttcacgcctcgagaccgggtacgtgacggtcgacgaggagaacggcgccgagctgttctactacttcatcgagtcggagggcgaccccagcaccgaccccgtcctcctctggatcaccggcggcgaccgctgctccgtcctcagcgccctcttcttcgagattgggccgttgaagctggtcatcgagccctacaacggcagcttgccgcggctgcactaccacccttactcgtggacaaaggtcgccagcatcctcttcgtcgactcgccggtcggcgccggcttctccttctcaagggaccctaaggggtacgacgtcggcgacgtctctgcttcgatgcagctcatcaagcttctcagggagtggttcactgagtatccgcactacctctcaaatccgttctacgttggaggagactcctatgccggaaagatcgtcccgtttatcgtgcagaagatctcggaagatattgaagctggcgtcagaccgacgtttaatctgaagggctatctggtaggtaatccgagtacaggggaacgcattgacctcgaatcaagagtgccgtacagccatggagtcggaatcatttcagatcagttatatgagatgataatggagcactgcgaaggtgaggattatgacaatcccagcaacgtgatttgccaacaagctttggctaggtttgacagcctactacacgaaggttcgagggcccaaattttgaacccaaattgcatttatgtgtcccccaagccaaatcatgagacaatcgacagaaagatcttgaagggggagcatggaggactaaaacatccacctcctcaaccttcaataaaatgtggtgtctatgccaactatttgtcatatttttgggcaaacaacaactttacacgaagaactctaggaatcaagaagggcaccataaatgaatgggttaggtgccacgaacatgatctaccatacaacatcgacatcaggagcagcataaagtatcatcgaaatgttacactaaaagtggtgaccacgatgctgttgtgccattcctcgggacacaagcctgggtga

SEQ ID NO: 148 - protein, 393 aa, Oryza sativa
MAVRLCCCCFLLFVTIAAAGGSLTRTNVATLPGFDGALPSRLETGYVTVDEENGAELFYYFIESEGDPSTDPVLLWITGGDRCSVLSALFFEIGPLKLVIEPYNGSLPRLHYHPYSWTKVASILFVDSPVGAGFSFSRDPKGYDVGDVSASMQLIKLLREWFTEYPHYLSNPFYVGGDSYAGKIVPFIVQKISEDIEAGVRPTFNLKGYLVGNPSTGERIDLESRVPYSHGVGIISDQLYEMIMEHCEGEDYDNPSNVICQQALARFDSLLHEGSRAQILNPNCIYVSPKPNHETIDRKILKGEHGGLKHPPPQPSIKCGVYANYLSYFWANNNFTRRTLGIKKGTINEWVRCHEHDLPYNIDIRSSIKYHRNVTLKVVTTMLLCHSSGHKPG

SEQ ID NO: 149 - DNA, 1476 bp, Zea mays
atgacgctggtgaggatgccttgccgcctctgctgcttcctgctcgtcgtcgccgcctccgccgtctccgggcgggggcgggtggtcaccaccctcccgggctacgagggccgcctccccttccacctcgaaacagggtatgtggaggtggacgaggacgccggcgcggagctcttctactacttcgtccgggccgagtccggcgccgacgacagcgacaccccgttcgtcctccggatccccggcggccaacgctgctccgccttcagcggcctcgcctacgagataggtcccatcatgttcgtcgtggagccctacaacggcagcttgccgcgcctgcgctacaacccaaactcgtggacaaaggtggcgcatattctttttgtggattcgccagttggggccgggttttccttctccagagatgccaaaggctacaacgctggagcggtatcaaccacgctgcacttggcgaagttcctcaacaagtggttcaacgaccatcctgagtaccatgcaaatcctttctacatcgatggagaatcatacgctggaaaaattgtaccattccttgcacagatgatttcagaaggtattggagcaggaatgaagtcggcacctcgtctcaagggctatctagtgggcaacccgtccacagaggagcgtattgatgttagcgctagagtgccttgtgctcacgggtttgggattatatcacatcagttgtatgagatgatattggggcattgccatggagaggactactccaatcctgcaaaagagttgtgtggtcaggcactgaagaccttcaatgatctcacctcggaagttgcgcaaggccatgtactgcaggagaaatgcgtcgcggcgtcgtcgtctcctgtgctgaacgcaaacagcagggtggccggcggctcgtcgtcgtgggcctcagatggcaggaagatcctacgggaggaagaaatggtgggacgacgcggagtactgggcaagctatttcatccaccggctcttcctccattcagttgcagagtttaccggtactacctgtcgtatttctgggcaaacgacaggcgcacccgggatgctcttgggatcaaggagggcaccgtggatgagtgggtcagatgccacaacgacgaccaagagttgccttacgagagcgagctgaagagtgtcgtcaagtaccaccggaacctgacgtccagaggctaccgagccatggtatacagcggcgaccatgacctgctggtgccacatctgggcacccaggcctgggtcaggtcgctcaacttccccgtcgtggacgactggagggcgtggcatctcggtggccagtcagctggatttacaataacttactcaaacaacatgacattcgcgacgatcaagggcgccgggcatacagcacctgagtacgagccggagaggtgtttcgccatgttcagccgttggatcctcaaccggccactctaa

SEQ ID NO: 150 - protein, 491 aa, Zea mays
MTLVRMPCRLCCFLLVVAASAVSGRGRVVTTLPGYEGRLPFHLETGYVEVDEDAGAELFYYFVRAESGADDSDTPFVLRIPGGQRCSAFSGLAYEIGPIMFVVEPYNGSLPRLRYNPNSWTKVAHILFVDSPVGAGFSFSRDAKGYNAGAVSTTLHLAKFLNKWFNDHPEYHANPFYIDGESYAGKIVPFLAQMISEGIGAGMKSAPRLKGYLVGNPSTEERIDVSARVPCAHGFGIISHQLYEMILGHCHGEDYSNPAKELCGQALKTFNDLTSEVAQGHVLQEKCVAASSSPVLNANSRVAGGSSSWASDGRKILREEEMVGRRGVLGKLFHPPALPPFSCRVYRYYLSYFWANDRRTRDALGIKEGTVDEWVRCHNDDQELPYESELKSVVKYHRNLTSRGYRAMVYSGDHDLLVPHLGTQAWVRSLNFPVVDDWRAWHLGGQSAGFTITYSNNMTFATIKGAGHTAPEYEPERCFAMFSRWILNRPL

SEQ ID NO: 151 - DNA, 1263 bp, Sorghum bicolor
atgaggacgatgatgccttgccgcctcagctgcttcctgctcgtcgtcgccgccgcctccggctctgctggctccgggcaggggcgggtggtcaccaccctcccgggcttcgagggccgcctccccttccacctcgaaacagggtacgtggaggtggacgaggacgccggcgcggagctcttctactacttcgtccagtccgagtccgagtccgccggcgacgcccctctcctcctctggctcaccggcggccaacgctgctccgccttgagcggcctcgcctacgagattggtcccatcaggttcgtcgtggagccctacgacggcaccttaccgcgcctgcgctacgactcgagaaactcgtggacaaaggtggcacatattctttttgttgattcgccagttggggctgggttttccttctccaaagatcccaaaggctactatgttggagacatatcatcctccatgcagttgcataagttcctcaacaagtggttcaacgagcatccggactaccttgcaaatcctttctacatcggtggagaatcatacgctggaaaaactgtaccattccttgcacagatgatttcagaaggtgttgaagcaggaatgaagtccgaacctaatctcaagggctatctagtgggcaacccgtccacagaggaacgtattgattttggctctagagtgcctcatgctcacggctttggaattatatcacatcagctgtatgagacgatatccgggcattgccaaggagaagactactccaatcctgcaaatgagttgtgtggtcaggctctgaataccttcaatgatagttactcctactccctgtcgtatttctgggcaaacgacagacgcacccgagatgctcttgggatcaaggagggtaccgtggatgagtgggtcagatgcgacgatgaagcagagctgccttacgagcgcgacctcaagagtgtcgtcaagtaccactggaacctgacctccagaggctaccgcgccctcgtattcagcggcgaccatgacctgatggtgccacacctgggcacccaggcctgggtcagatctctcaacttccccatcgtcgacgactggagggcctggcatcttggtgggcagtcagctggattcacaatcagttactcaaacaacatgacattcgcgacgatcaagggcggcggacatacagcacctgagtacgagccggagaggtgtttcgccatgttcagccgctgggtgctcaaccggccactctaa

SEQ ID NO: 152 - protein, 420 aa, Sorghum bicolor
MRTMMPCRLSCFLLVVAAASGSAGSGQGRVVTTLPGFEGRLPFHLETGYVEVDEDAGAELFYYFVQSESESAGDAPLLLWLTGGQRCSALSGLAYEIGPIRFVVEPYDGTLPRLRYDSRNSWTKVAHILFVDSPVGAGFSFSKDPKGYYVGDISSSMQLHKFLNKWFNEHPDYLANPFYIGGESYAGKTVPFLAQMISEGVEAGMKSEPNLKGYLVGNPSTEERIDFGSRVPHAHGFGIISHQLYETISGHCQGEDYSNPANELCGQALNTFNDSYSYSLSYFWANDRRTRDALGIKEGTVDEWVRCDDEAELPYERDLKSVVKYHWNLTSRGYRALVFSGDHDLMVPHLGTQAWVRSLNFPIVDDWRAWHLGGQSAGFTISYSNNMTFATIKGGGHTAPEYEPERCFAMFSRWVLNRPL

SEQ ID NO: 153 - DNA, 1314 bp, Arabidopsis thaliana
atggggaaagagtgctactacttgtcatggatactcaagtttcatcttctacttgttttgattcagcttgttgattcaggatctaccatcagatttcttcctggtttccaaggccctcttcctttcgagctcgagaccgggtacattggtgtgggtgaggcagagaaagatcaaatgttctactacttcatcaaatctgagagtaatccagagaaagaccctcttcttctctggttaagtggaggtcctttctgttcttcgttcactgctcttatttatgagaatgggccaattgctttcaaggctgaggaatacaatggaagtatcccctctttggtctctaccacatatgcatggactaaggtggcgagcattctctatttggatcagcctgttgggactggcttctcctactccagaaatccacttgctgatataccaagtgacacaggggtagctaagccggtcaacgagtttcttcataagtggctagacaaacatcctgagttcttatccaatccgctctatgtcgccggaaattcttattccggtattgtcattccgaccatcgttcaagaaatctcaaatggaaaccatttagacagcaaacctcaaataaatcttcagggctttgtactcggaaacccggcaacagacactgatattgatcttaactctcgcattccatttgctcatggaaaggcactgatctctgatgaacactatgagtctctaaagagaagttgtcaagggaattatattagcgtgaatcctcgtaacacaaaatgcttgaaactccttgaagactttaaaaagtgtgtttctggaataagtgaagaatatattctaaaacctgactgcatgtggctttattcttgtatggcaaatctgcattcgctatctgaatattgggcaaatgagaaaagcgtgcgcaaagctcttctagtgaacgagggtaccgtaagaaaatggatacgatgtaatacggaaatcgcatacaataaggacattaggagtagtgtgccatatcataagtatattagcattgaagggtatcgatctctcgtcttcagtggtgatcacgacatgttagtacctttccttggaactcaagcatggataagatctctcaactattccattgttgacgattggagaccatggatggttcagaatcaagttgctggatacacaaggacttatgcgaataagatgacatttgccactgtcaaaggaggcgggcacacgagtgagtataaaccagtggagacctatatcatgatcaagaggtggttaagtggtcaacctctataa

SEQ ID NO: 154 - protein, 437 aa, Arabidopsis thaliana
MGKECYYLSWILKFHLLLVLIQLVDSGSTIRFLPGFQGPLPFELETGYIGVGEAEKDQMFYYFIKSESNPEKDPLLLWLSGGPFCSSFTALIYENGPIAFKAEEYNGSIPSLVSTTYAWTKVASILYLDQPVGTGFSYSRNPLADIPSDTGVAKPVNEFLHKWLDKHPEFLSNPLYVAGNSYSGIVIPTIVQEISNGNHLDSKPQINLQGFVLGNPATDTDIDLNSRIPFAHGKALISDEHYESLKRSCQGNYISVNPRNTKCLKLLEDFKKCVSGISEEYILKPDCMWLYSCMANLHSLSEYWANEKSVRKALLVNEGTVRKWIRCNTEIAYNKDIRSSVPYHKYISIEGYRSLVFSGDHDMLVPFLGTQAWIRSLNYSIVDDWRPWMVQNQVAGYTRTYANKMTFATVKGGGHTSEYKPVETYIMIKRWLSGQPL

SEQ ID NO: 155 - DNA, 1410 bp, Glycine max
atggaaaattttagttcaagttacatttattactgggttctactacccttttttttgttatcacaattctcctttcaacttgcatggtgtggctccatagtaaagttccttcctggattcaagggaccccttccttttgtacttgaaaccgggtatgtgggagtgggtgaatcagaggatgtgcaggcattctactacttcattgagtcagagaacaatcccaagaaagatcctctcatgctttggctcactggtggccctggttgctcagccttatctggacttgtgtttgaaataggaccacttacattcaaatatgaggaatacaatgggagcctgcccaatttggtcttgaggccacactcatggacaaaggttagtagcattatatttgtagacttgcctgtttccacgggcttcacttatgccacaacagagtttgctgctcaacgaagcgactggattctagttcaccaagtccatcagtttcttaggaagtggttgattgatcatccaaatttttcgtcaaatgaagtttacattggtggcgattcatactctggcattcctattccagtgattgttcaagaaatttcacgaggaaatgaaaaagggctccaaccatggataaatctccagggatacctgctgggaaatgcagcaacaactcgaagggaaaaaaactatcaaattcccttcgctcatggaatgggacttatttctgatgaactatatgggtcactgcaaaaaaattgtaaagaagagtacataaatgtagacaccagaaatgtattatgttctagagatatcgagtcattcaatgaggttacatcaggacttaattcagcccatattttggacccgtcatgtgagtggcttgatactgaaacatcttggaggagatctctacttaagaaatatcccagaaagaatttccttaatactcacctcaaattggcacccttaaactgtcggagttatgtatacttcctctgcggttattgggccaatgatgataatgttcgcactgcactgcacatccgtaagggaagtataggaaaatggcatcgttgtaccttcgatatacctaacaagaaggatatctcaagcagctatgagtatcatgtaaatctcagtagaaaaggctaccgttcgctgatatacagtggcgatcatgacatgacaattcctttcttggcaactcaagcatggataagatctttaaactactccattgtggatgagtggaggcaatggcatacaaatggtcaagttgcaggatacacaaggacttactccaatcggatgacatttgcaactgtgaagggaggaggccacacagctccggagtacaagcctgatgaatgctttgccatgttcagtaggtggatatctaacagtgctttgtag

SEQ ID NO: 156 - protein, 469 aa, Glycine max
MENFSSSYIYYWVLLPFFLLSQFSFQLAWCGSIVKFLPGFKGPLPFVLETGYVGVGESEDVQAFYYFIESENNPKKDPLMLWLTGGPGCSALSGLVFEIGPLTFKYEEYNGSLPNLVLRPHSWTKVSSIIFVDLPVSTGFTYATTEFAAQRSDWILVHQVHQFLRKWLIDHPNFSSNEVYIGGDSYSGIPIPVIVQEISRGNEKGLQPWINLQGYLLGNAATTRREKNYQIPFAHGMGLISDELYGSLQKNCKEEYINVDTRNVLCSRDIESFNEVTSGLNSAHILDPSCEWLDTETSWRRSLLKKYPRKNFLNTHLKLAPLNCRSYVYFLCGYWANDDNVRTALHIRKGSIGKWHRCTFDIPNKKDISSSYEYHVNLSRKGYRSLIYSGDHDMTIPFLATQAWIRSLNYSIVDEWRQWHTNGQVAGYTRTYSNRMTFATVKGGGHTAPEYKPDECFAMFSRWISNSAL

SEQ ID NO: 157 - DNA, 1038 bp, Oryza sativa
atgaagaatcgtattctggagggagctcttatgtgccagatttttcaggttagttataagctgagagaaggaaatagagtagctcatagacttgctgatttggggtcctttgtgcctgttatagtgaagtttactaggctcaaaatctttctggttccgctactgatcagacttgatgaggatggctctgaattaagtgatcggaatggcaagaagatgaatgagccgagacgatgccgccacgcaaatgacgttgcgtgcgtgcgtgacgccgccgccgaaacccacgctggaatttttactaccgccagaactcctgctcaagatcgtcgctcgctcggacgctgccacactcgtccgctcccagaggacgatgcgactgaggaggcggacaaggcggcgacggcgggggatacggacggtgacggagggggcggacaaggcggtgacggcggggatacggacgatgacggaggggacggacaaggcggcgaggccgggagaggaatagcgaggaggcatcaggaattgtcatttcgccgagatggcgcacgcaacactgcccagagatttcgaaaccaaagcacactagatctggccctgccgccgccagagctgaccggcgtctacgcggcggcggggaggtgggaggcggaggtgcggaggtggcggccagaggcagccgggcctgcgcctggccgtgtggattggggatggctggaggtggtcgggagggcggtggagagtggcaagacgttagtgatggagtccctctggatgacggtaggggcggcgaagcacggctgtggcctagtggcagcggctcccgtcggcgccaatggccctaggggctgcggcggcccaggagggcggcgtctcccgtcgccgccggcagccctaggggctgctgcggaagacggcgttggtgccggtggcccagggcagcggagcactgtgaatccgatgaagtggaagccgatcccgtcggttcgtgtatggttgactagttccagccaaacgacgaatgacgacggttga

SEQ ID NO: 158 - protein, 345 aa, Oryza sativa
MKNRILEGALMCQIFQVSYKLREGNRVAHRLADLGSFVPVIVKFTRLKIFLVPLLIRLDEDGSELSDRNGKKMNEPRRCRHANDVACVRDAAAETHAGIFTTARTPAQDRRSLGRCHTRPLPEDDATEEADKAATAGDTDGDGGGGQGGDGGDTDDDGGDGQGGEAGRGIARRHQELSFRRDGARNTAQRFRNQSTLDLALPPPELTGVYAAAGRWEAEVRRWRPEAAGPAPGRVDWGWLEVVGRAVESGKTLVMESLWMTVGAAKHGCGLVAAAPVGANGPRGCGGPGGRRLPSPPAALGAAAEDGVGAGGPGQRSTVNPMKWKPIPSVRVWLTSSSQTTNDDG